(19) United States
(12) Patent Application Publication
(10) Pub. No.: US 2016/0361298 A1
(43) Pub. Date: Dec. 15, 2016

(54) METHODS AND COMPOSITIONS FOR TREATING CANCER

(71) Applicant: Globavir BioSciences, Inc., Los Altos, CA (US)

(72) Inventors: Paul Novick, San Francisco, CA (US); Jonathan Choy, Lafayette, CA (US); Sumit Mahajan, Foster City, CA (US); Ritika Prasad, Mountain View, CA (US); Shalabh Gupta, Mountain View, CA (US)

(21) Appl. No.: 15/179,764

(22) Filed: Jun. 10, 2016

Related U.S. Application Data
(60) Provisional application No. 62/174,430, filed on Jun. 11, 2015.
(60) Provisional application No. 62/174,424, filed on Jun. 11, 2015.
(60) Provisional application No. 62/188,413, filed on Jul. 2, 2015.
(60) Provisional application No. 62/195,750, filed on Jul. 22, 2015.

(51) Int. Cl. A61K 31/4174; A61K 31/4184; A61K 31/496; A61K 39/395; A61K 31/4196; A61K 31/4152; A61K 45/06; C07K 16/28; A61K 31/4178; A61K 31/167

(52) U.S. Cl. CPC A61K 31/4174; A61K 31/4178; A61K 31/4184; A61K 31/496; A61K 31/167; A61K 31/4196; A61K 31/4152; A61K 45/06; C07K 16/2818; A61K 39/3955; A61K 2039/505

(57) ABSTRACT

Described are compositions and methods for treating cancer. Some methods comprise the administration of an effective amount of at least one inhibitor of tryptophan 2,3-dioxygenase (TDO) and/or indoleamine 2,3-dioxygenase (IDO), optionally combined with one or more additional anti-cancer agents.

### CROSS-REFERENCE TO RELATED APPLICATIONS

This application claims the benefit of U.S. Provisional Application No. 62/174,430 filed Jun.11, 2015; U.S. Provisional Application No. 62/174,424 filed Jun. 11, 2015; U.S. Provisional Application No. 62/188,413 filed Jul. 2, 2015; and U.S. Provisional Application No. 62/195,750 filed Jul. 22, 2015; the entirety of which are incorporated by reference herein.

### BACKGROUND

Immuno-oncology focuses on the development and delivery of therapies that improve a body's intrinsic potential for generating an effective immune response against cancer. Immuno-oncology therapies often target the immune system and not the cancer, providing the potential to treat cancer across a variety of tumor types. Manipulation of T-cell modulation is one method of augmenting the immune system to treat cancer.

### SUMMARY OF THE INVENTION

The immune system has an intrinsic potential for recognizing and eliminating tumor cells. However, tumors can adapt and circumvent the immune system by disrupting T-cell checkpoint pathways. An approach to disrupt tumor inhibition of the immune system involves the targeted block of checkpoint pathways Immune checkpoint pathway targets for activating therapeutic antitumor immunity include programmed cell death protein (PD-1) and its ligand PD-L1, indoleamine 2,3-dioxygenase (IDO), and cytotoxic T-lymphocyte antigen 4 (CTLA-4). IDO is a heme-containing enzyme that catalyzes the oxidation of L-tryptophan to N-formyl-L-kynurenine. Another heme-containing enzyme that also catalyzes tryptophan degradation is tryptophan 2,3-dioxygenase (TDO). TDO has been found to be expressed in many tumors and this expression prevents tumor rejection by locally depleting tryptophan. Development of TDO inhibitors for the treatment of tumors is an active area of drug development. As the active sites of both IDO and TDO enzymes are structurally similar, various inhibitors of TDO and IDO have activity against one another. Provided in this disclosure are compounds and compositions comprising TDO inhibitors, IDO inhibitors, and inhibitors of both TDO and IDO for active stimulation of the immune system. In various aspects, provided are TDO inhibitors, IDO inhibitors, and inhibitors of both TDO and IDO that are useful for the treatment of cancer.

In one aspect, provided herein is a method of treating cancer comprising administering to an individual in need thereof an effective amount of econazole, sulconazole, isoconazole, miconazole, sertaconazole, tioconazole, fenticonazole, liarozole, cloconazole, itraconazole, niclosamide, deferasirox, eltrombopag, or a pharmaceutically acceptable salt, solvate, or combination thereof. In some embodiments, econazole or a pharmaceutically acceptable salt or solvate of econazole is administered to the individual. In some embodiments, sulconazole or a pharmaceutically acceptable salt or solvate of sulconazole is administered to the individual. In some embodiments, isoconazole or a pharmaceutically acceptable salt or solvate of isoconazole is administered to the individual. In some embodiments, miconazole or a pharmaceutically acceptable salt or solvate of miconazole is administered to the individual. In some embodiments, sertaconazole or a pharmaceutically acceptable salt or solvate of sertaconazole is administered to the individual. In some embodiments, tioconazole or a pharmaceutically acceptable salt or solvate of tioconazole is administered to the individual. In some embodiments, fenticonazole or a pharmaceutically acceptable salt or solvate of fenticonazole is administered to the individual. In some embodiments, liarozole or a pharmaceutically acceptable salt or solvate of liarozole is administered to the individual. In some embodiments, cloconazole or a pharmaceutically acceptable salt or solvate of cloconazole is administered to the individual. In some embodiments, itraconazole or a pharmaceutically acceptable salt or solvate of itraconazole is administered to the individual. In some embodiments, niclosamide or a pharmaceutically acceptable salt or solvate of niclosamide is administered to the individual. In some embodiments, deferasirox or a pharmaceutically acceptable salt or solvate of deferasirox is administered to the individual. In some embodiments, eltrombopag or a pharmaceutically acceptable salt or solvate of eltrombopag is administered to the individual. In some embodiments, the cancer comprises colon cancer, pancreatic cancer, cutaneous T-cell lymphoma, glioma, head and neck cancer, hepatocarcinoma, leukemia, glioblastoma, colorectal cancer, gallbladder, mastocytoma, acute myeloid leukemia, adrenocortical cancer, bladder urothelial cancer, brain tumor, brain lower grade glioma, breast cancer, breast invasive cancer, cervical cancer, cholangiocarcinoma, cutaneous melanoma, diffuse large B-cell lymphoma, endometrial cancer, glioblastoma multiform, H&N squamous cell carcinoma, hepatocellular carcinoma, kidney chromophobe carcinoma, lung cancer, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate cancer, rectum adenocarcinoma, rectum cancer, renal cell cancer, renal papillary cell cancer, sarcoma, testicular germ cell tumors, thymoma, thyroid cancer, uterine carcinosarcoma, melanoma, uveal melanoma, or a combination thereof. In some embodiments, the effective amount is from about 5 mg to about 5,000 mg. In some embodiments, the individual is administered an additional active agent comprising a PD-1/PD-L pathway inhibitor, IDO inhibitor, TDO inhibitor, CTLA-4 inhibitor, or a combination thereof. In some embodiments, the individual is administered an additional active agent comprising BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or a salt, solvate or combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of inhibiting tryptophan 2,3-dioxygenase (TDO), indoleamine 2,3-dioxygenase (IDO), or both TDO and IDO in an individual in need thereof, the method comprising administering to the individual an effective amount of: an antifungal agent, antiparasitic agent, iron chelator, thrombopoietin (TPO) receptor agonist, or a combination thereof. In some embodiments, the antifungal agent comprises tioconazole, liarozole, sertaconazole, econazole, sulconazole, miconazole, isoconazole, itraconazole, fenticonazole, cloconazole; or a pharmaceutically acceptable salt, solvate or combination thereof. In some embodiments, the antiparasitic agent comprises niclosamide or a pharmaceutically acceptable salt or solvate of niclosamide. In some embodiments, the iron chelator comprises deferasirox or a pharmaceutically acceptable salt or solvate of deferasirox. In some embodiments, the TPO receptor agonist comprises eltrombopag or a pharmaceutically acceptable salt or solvate of eltrombopag. In some embodiments, the individual has cancer. In some embodiments, the cancer comprises colon cancer, pancreatic cancer, cutaneous T-cell lymphoma, glioma, head and neck cancer, hepatocarcinoma, leukemia, glioblastoma, colorectal cancer, gallbladder, mastocytoma, acute myeloid leukemia, adrenocortical cancer, bladder urothelial cancer, brain tumor, brain lower grade glioma, breast cancer, breast invasive cancer, cervical cancer, cholangiocarcinoma, cutaneous melanoma, diffuse large B-cell lymphoma, endometrial cancer, glioblastoma multiform, H&N squamous cell carcinoma, hepatocellular carcinoma, kidney chromophobe carcinoma, lung cancer, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate cancer, rectum adenocarcinoma, rectum cancer, renal cell cancer, renal papillary cell cancer, sarcoma, testicular germ cell tumors, thymoma, thyroid cancer, uterine carcinosarcoma, melanoma, uveal melanoma, or a combination thereof. In some embodiments, the effective amount is from about 5 mg to about 5,000 mg. In some embodiments, the individual is administered an additional active agent comprising a PD-1/PD-L pathway inhibitor, IDO inhibitor, TDO inhibitor, CTLA-4 inhibitor, or a combination thereof. In some embodiments, the individual is administered an additional active agent comprising BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or a salt, solvate or combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In another aspect, provided herein is a composition comprising: (a) an antifungal agent, antiparasitic agent, iron chelator, thrombopoietin (TPO) receptor agonist, or a combination thereof; and (b) an anti-cancer agent. In some embodiments, the anti-cancer agent is a component of an immunotherapy. In some embodiments, the anti-cancer agent is a component of a chimeric antigen receptor (CAR) T-cell therapy. In some embodiments, the anti-cancer agent comprises a PD-1/PD-L pathway inhibitor, IDO inhibitor, TDO inhibitor, CTLA-4 inhibitor, or a combination thereof. In some embodiments, the anti-cancer agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or a salt, solvate or combination thereof.

In one aspect, provided herein is a method of treating cancer comprising administering to an individual in need thereof an effective amount of a compound of Formula (VIII), or a pharmaceutically acceptable salt or solvate thereof:

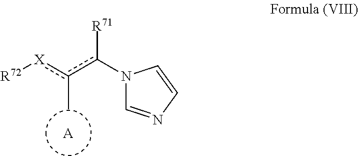

wherein
each are independently a single bond or a double bond provided that they are not both double bonds;
Ring A is optionally substituted aryl, or optionally substituted heteroaryl;
X is O or S, when is; or
X is N, when is;
R71 is hydrogen or optionally substituted C1-C6 alkyl;
R72 is —(C1-C6 alkylene) (optionally substituted aryl), —(C1-C6 alkylene) (optionally substituted heteroaryl), —(C1-C6 alkylene)-O-(optionally substituted aryl), or —O—(C1-C6 alkylene) (optionally substituted aryl), provided that when R2 is —O—(C1-C6 alkylene) (optionally substituted aryl), then X is N.

In some embodiments, the compound of Formula (VIII) or a pharmaceutically acceptable salt or solvate thereof comprises:

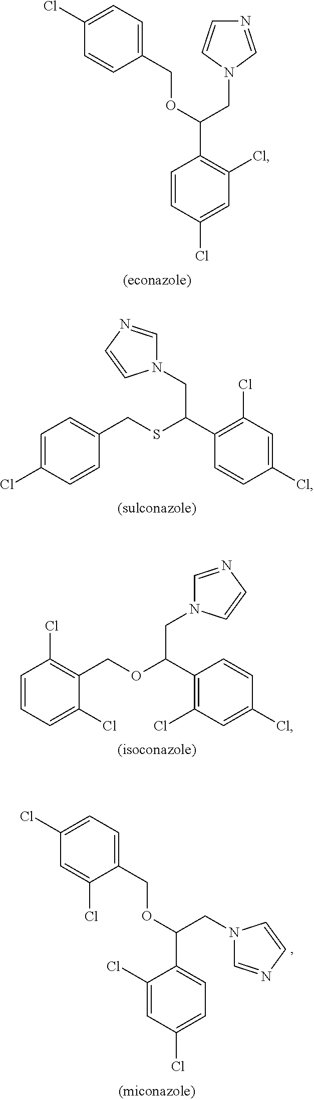 [SMILES: Clc1ccc(COC(Cn2ccnc2)c2ccc(Cl)cc2Cl)cc1 ; Clc1ccc(CSC(Cn2ccnc2)c2ccc(Cl)cc2Cl)cc1 ; Clc1ccc(C(Cn2ccnc2)OCc2c(Cl)cccc2Cl)c(Cl)c1 ; Clc1ccc(COC(Cn2ccnc2)c2ccc(Cl)cc2Cl)c(Cl)c1]

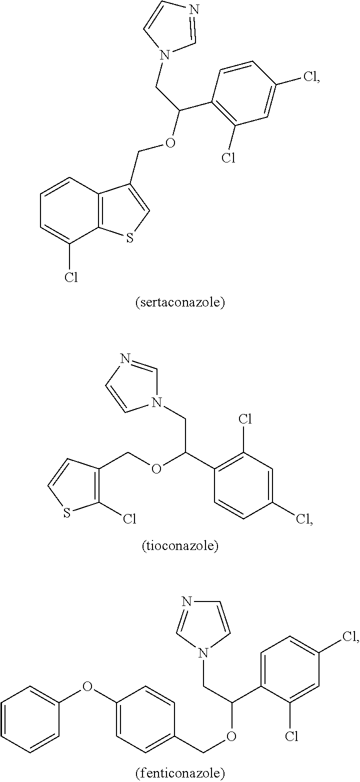 [SMILES: Clc1ccc(C(Cn2ccnc2)OCc2csc3c(Cl)cccc23)c(Cl)c1 ; Clc1ccc(C(Cn2ccnc2)OCc2ccsc2Cl)c(Cl)c1 ; Clc1ccc(C(Cn2ccnc2)OCc2ccc(Oc3ccccc3)cc2)c(Cl)c1]

or a combination thereof.

In some embodiments, the compound of Formula (VIII) or a pharmaceutically acceptable salt or solvate thereof comprises econazole. In some embodiments, the compound of Formula (VIII) or a pharmaceutically acceptable salt or solvate thereof comprises sulconazole. In some embodiments, the compound of Formula (VIII) or a pharmaceutically acceptable salt or solvate thereof comprises isoconazole. In some embodiments, the compound of Formula (VIII) or a pharmaceutically acceptable salt or solvate thereof comprises miconazole. In some embodiments, the compound of Formula (VIII) or a pharmaceutically acceptable salt or solvate thereof comprises sertaconazole. In some embodiments, the compound of Formula (VIII) or a pharmaceutically acceptable salt or solvate thereof comprises tioconazole. In some embodiments, the compound of Formula (VIII) or a pharmaceutically acceptable salt or solvate thereof comprises fenticonazole. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a combination comprising (a) a compound of Formula (VIII), or a pharmaceutically acceptable salt or solvate thereof:

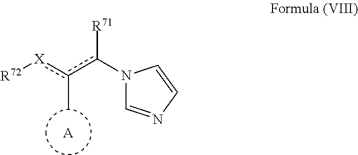

wherein
each [symbol] are independently a single bond or a double bond provided that they are not both double bonds;
Ring A is optionally substituted aryl, or optionally substituted heteroaryl;
X is O or S, when

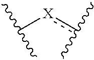

is

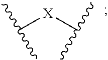

or

X is N, when

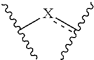

is

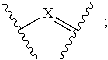

R⁷¹ is hydrogen or optionally substituted C₁-C₆ alkyl;
R⁷² is —(C₁-C₆ alkylene) (optionally substituted aryl), —(C₁-C₆ alkylene) (optionally substituted heteroaryl), —(C₁-C₆ alkylene)-O-(optionally substituted aryl), or —O—(C₁-C₆ alkylene) (optionally substituted aryl), provided that when R² is —O—(C₁-C₆ alkylene) (optionally substituted aryl), then X is N;
and
(b) an additional active agent.

In some embodiments, the compound of Formula (VIII) or a pharmaceutically acceptable salt or solvate thereof comprises:

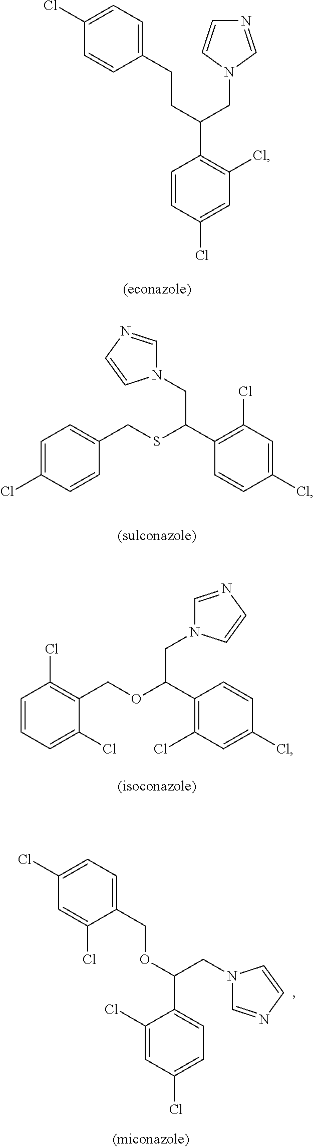 [SMILES: Clc1ccc(CCC(Cn2ccnc2)c2ccc(Cl)cc2Cl)cc1 ; Clc1ccc(CSC(Cn2ccnc2)c2ccc(Cl)cc2Cl)cc1 ; Clc1ccc(C(Cn2ccnc2)OCc2c(Cl)cccc2Cl)c(Cl)c1 ; Clc1ccc(COC(Cn2ccnc2)c2ccc(Cl)cc2Cl)c(Cl)c1]

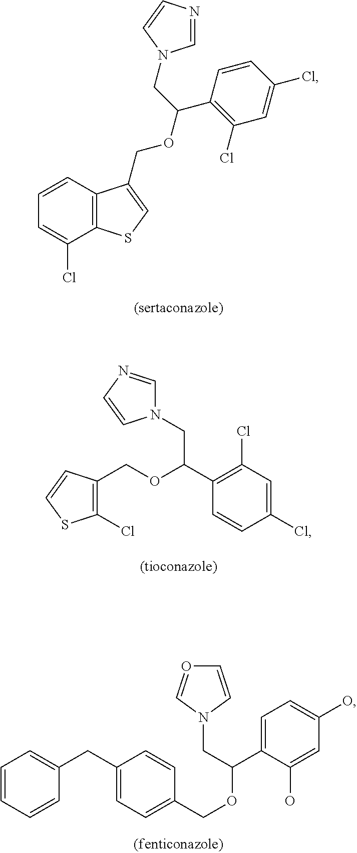

or a combination thereof.

In some embodiments, the compound of Formula (VIII) or a pharmaceutically acceptable salt or solvate thereof comprises econazole. In some embodiments, the compound of Formula (VIII) or a pharmaceutically acceptable salt or solvate thereof comprises sulconazole. In some embodiments, the compound of Formula (VIII) or a pharmaceutically acceptable salt or solvate thereof comprises isoconazole. In some embodiments, the compound of Formula (VIII) or a pharmaceutically acceptable salt or solvate thereof comprises miconazole. In some embodiments, the compound of Formula (VIII) or a pharmaceutically acceptable salt or solvate thereof comprises sertaconazole. In some embodiments, the compound of Formula (VIII) or a pharmaceutically acceptable salt or solvate thereof comprises tioconazole. In some embodiments, the compound of Formula (VIII) or a pharmaceutically acceptable salt or solvate thereof comprises fenticonazole. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises an immunotherapy agent, a stem cell therapy agent, a CAR T-cell therapy agent, BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof.

In one aspect, provided herein is a method of treating cancer comprising administering to an individual in need thereof an effective amount of a compound or a pharmaceutically acceptable salt or solvate thereof, the compound comprising:

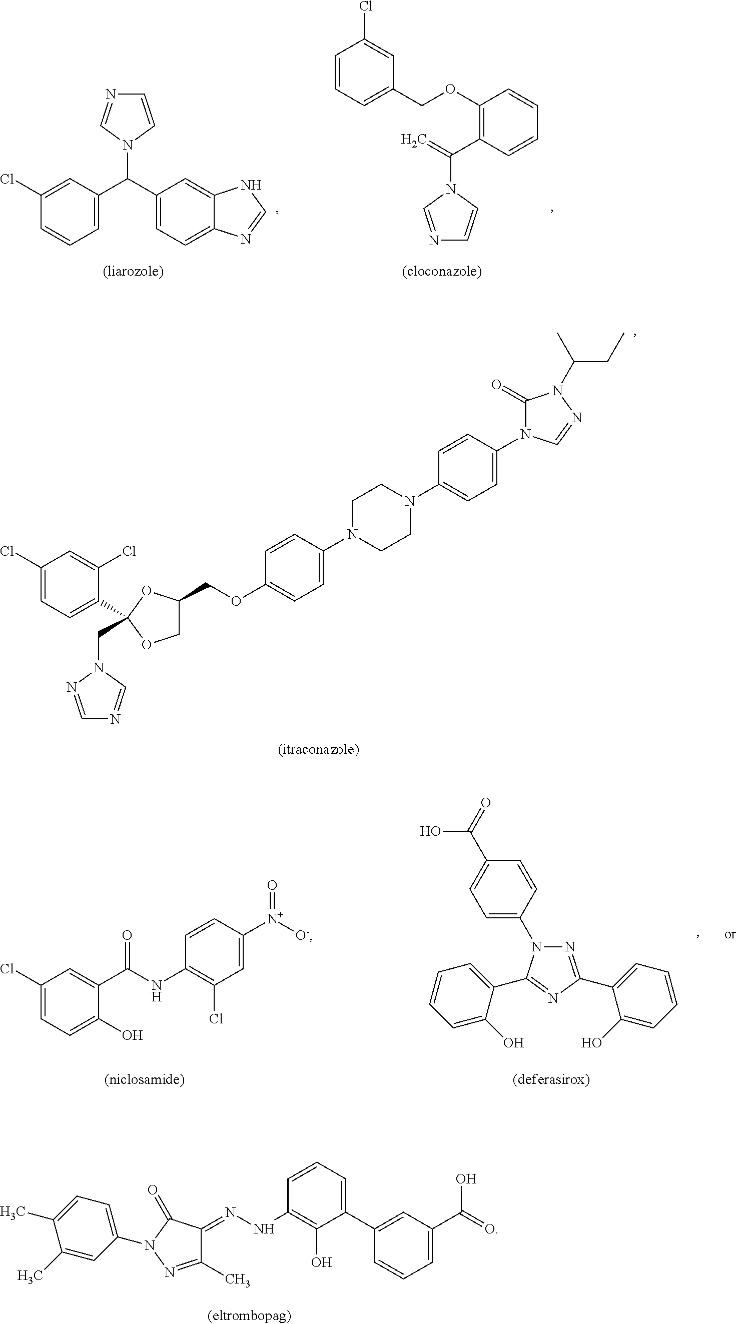

In some embodiments, the compound comprises liarozole. In some embodiments, the compound comprises cloconazole. In some embodiments, the compound comprises itraconazole. In some embodiments, the compound comprises niclosamide. In some embodiments, the compound comprises deferasirox. The compound comprises eltrombopag. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of tioconazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of liarozole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of sertaconazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of econazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of sulconazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of miconazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of isoconazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of itraconazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of niclosamide or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of deferasirox or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of fenticonazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of cloconazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of eltrombopag or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, antiparasitic agent, iron chelator, thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of an antifungal agent comprising tioconazole, liarozole, sertaconazole, econazole, sulconazole, miconazole, isoconazole, itraconazole, fenticonazole, cloconazole, or salts, solvates or combinations thereof. In some embodiments, the antifungal agent comprises tioconazole or a pharmaceutically acceptable salt or solvate thereof. In some embodiments, the antifungal agent comprises liarozole or a pharmaceutically acceptable salt or solvate thereof. In some embodiments, the antifungal agent comprises sertaconazole or a pharmaceutically acceptable salt or solvate thereof. In some embodiments, the antifungal agent comprises econazole or a pharmaceutically acceptable salt or solvate thereof. In some embodiments, the antifungal agent comprises sulconazole or a pharmaceutically acceptable salt or solvate thereof. In some embodiments, the antifungal agent comprises miconazole or a pharmaceutically acceptable salt or solvate thereof. In some embodiments, the antifungal agent comprises isoconazole or a pharmaceutically acceptable salt or solvate thereof. In some embodiments, the antifungal agent comprises itraconazole or a pharmaceutically acceptable salt or solvate thereof. In some embodiments, the antifungal agent comprises fenticonazole or a pharmaceutically acceptable salt or solvate thereof. In some embodiments, the antifungal agent comprises cloconazole or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises a second antifungal agent, an antiparasitic agent, an iron chelator, a thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of an antiparasitic agent. In some embodiments, the antiparasitic agent comprises niclosamide or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, a second antiparasitic agent, an iron chelator, a thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of an iron chelator. In some embodiments, the iron chelator comprises deferasirox or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, an antiparasitic agent, a second iron chelator, a thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of treating cancer comprising administering to an individual in need thereof an effective amount of a thrombopoietin (TPO) receptor agonist. In some embodiments, the TPO receptor agonist comprises eltrombopag or a pharmaceutically acceptable salt or solvate thereof. In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof. In some embodiments, the individual is administered an additional active agent. In some embodiments, the additional active agent is an anti-cancer agent. In some embodiments, the additional active agent comprises a PD-1/PD-L pathway inhibitor, IDO inhibitor, TDO inhibitor, CTLA-4 inhibitor, or a combination thereof. In some embodiments, the additional cancer agent comprises an antifungal agent, an antiparasitic agent, an iron chelator, a second thrombopoietin (TPO) receptor agonist, or a combination thereof. In some embodiments, the additional active agent comprises BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a combination comprising: (a) tioconazole; and (b) an anti-cancer agent. In another aspect, provided herein is a combination comprising: (a) tioconazole; and (b) a PD-1/PD-L pathway inhibitor, IDO inhibitor, TDO inhibitor, CTLA-4 inhibitor, or a combination thereof. In another aspect, provided herein is a combination comprising (a) tioconazole; and (b) an antifungal agent, an antiparasitic agent, an iron chelator, a thrombopoietin (TPO) receptor agonist, or a combination thereof. In another aspect, provided herein is a combination comprising (a) tioconazole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) tioconazole; and (b) liarozole, sertaconazole, econazole, sulconazole, miconazole, isoconazole, itraconazole, niclosamide, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) liarozole; and (b) an anti-cancer agent. In another aspect, provided herein is a combination comprising: (a) liarozole; and (b) a PD-1/PD-L pathway inhibitor, IDO inhibitor, TDO inhibitor, CTLA-4 inhibitor, or a combination thereof. In another aspect, provided herein is a combination comprising (a) liarozole; and (b) an antifungal agent, an antiparasitic agent, an iron chelator, a thrombopoietin (TPO) receptor agonist, or a combination thereof. In another aspect, provided herein is a combination comprising (a) liarozole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) liarozole; and (b) tioconazole, sertaconazole, econazole, sulconazole, miconazole, isoconazole, itraconazole, niclosamide, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) sertaconazole; and (b) an anti-cancer agent. In another aspect, provided herein is a combination comprising: (a) sertaconazole; and (b) a PD-1/PD-L pathway inhibitor, IDO inhibitor, TDO inhibitor, CTLA-4 inhibitor, or a combination thereof. In another aspect, provided herein is a combination comprising (a) sertaconazole; and (b) an antifungal agent, an antiparasitic agent, an iron chelator, a thrombopoietin (TPO) receptor agonist, or a combination thereof. In another aspect, provided herein is a combination comprising (a) sertaconazole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) sertaconazole; and (b) tioconazole, liarozole, econazole, sulconazole, miconazole, isoconazole, itraconazole, niclosamide, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) econzole; and (b) an anti-cancer agent. In another aspect, provided herein is a combination comprising: (a) econzole; and (b) a PD-1/PD-L pathway inhibitor, IDO inhibitor, TDO inhibitor, CTLA-4 inhibitor, or a combination thereof. In another aspect, provided herein is a combination comprising (a) econzole; and (b) an antifungal agent, an antiparasitic agent, an iron chelator, a thrombopoietin (TPO) receptor agonist, or a combination thereof. In another aspect, provided herein is a combination comprising (a) econzole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) econzole; and (b) tioconazole, liarozole, sertaconazole, sulconazole, miconazole, isoconazole, itraconazole, niclosamide, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) sulconazole; and (b) an anti-cancer agent. In another aspect, provided herein is a combination comprising: (a) sulconazole; and (b) a PD-1/PD-L pathway inhibitor, IDO inhibitor, TDO inhibitor, CTLA-4 inhibitor, or a combination thereof. In another aspect, provided herein is a combination comprising (a) sulconazole; and (b) an antifungal agent, an antiparasitic agent, an iron chelator, a thrombopoietin (TPO) receptor agonist, or a combination thereof. In another aspect, provided herein is a combination comprising (a) sulconazole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) sulconazole; and (b) tioconazole, liarozole, sertaconazole, econazole, miconazole, isoconazole, itraconazole, niclosamide, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) miconazole; and (b) an anti-cancer agent. In another aspect, provided herein is a combination comprising: (a) miconazole; and (b) a PD-1/PD-L pathway inhibitor, IDO inhibitor, TDO inhibitor, CTLA-4 inhibitor, or a combination thereof. In another aspect, provided herein is a combination comprising (a) miconazole; and (b) an antifungal agent, an antiparasitic agent, an iron chelator, a thrombopoietin (TPO) receptor agonist, or a combination thereof. In another aspect, provided herein is a combination comprising (a) miconazole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) miconazole; and (b) tioconazole, liarozole, sertaconazole, econazole, sulconazole, isoconazole, itraconazole, niclosamide, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) isoconazole; and (b) an anti-cancer agent. In another aspect, provided herein is a combination comprising: (a) isoconazole; and (b) a PD-1/PD-L pathway inhibitor, IDO inhibitor, TDO inhibitor, CTLA-4 inhibitor, or a combination thereof. In another aspect, provided herein is a combination comprising (a) isoconazole; and (b) an antifungal agent, an antiparasitic agent, an iron chelator, a thrombopoietin (TPO) receptor agonist, or a combination thereof. In another aspect, provided herein is a combination comprising (a) isoconazole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) isoconazole; and (b) tioconazole, liarozole, sertaconazole, econazole, sulconazole, miconazole, itraconazole, niclosamide, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) itraconazole; and (b) an anti-cancer agent. In another aspect, provided herein is a combination comprising: (a) itraconazole; and (b) a PD-1/PD-L pathway inhibitor, IDO inhibitor, TDO inhibitor, CTLA-4 inhibitor, or a combination thereof. In another aspect, provided herein is a combination comprising (a) itraconazole; and (b) an antifungal agent, an antiparasitic agent, an iron chelator, a thrombopoietin (TPO) receptor agonist, or a combination thereof. In another aspect, provided herein is a combination comprising (a) itraconazole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) itraconazole; and (b) tioconazole, liarozole, sertaconazole, econazole, sulconazole, miconazole, isoconazole, niclosamide, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) niclosamide; and (b) an anti-cancer agent. In another aspect, provided herein is a combination comprising: (a) niclosamide; and (b) a PD-1/PD-L pathway inhibitor, IDO inhibitor, TDO inhibitor, CTLA-4 inhibitor, or a combination thereof. In another aspect, provided herein is a combination comprising (a) niclosamide; and (b) an antifungal agent, an antiparasitic agent, an iron chelator, a thrombopoietin (TPO) receptor agonist, or a combination thereof. In another aspect, provided herein is a combination comprising (a) niclosamide; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) niclosamide; and (b) tioconazole, liarozole, sertaconazole, econazole, sulconazole, miconazole, isoconazole, itraconazole, deferasirox, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) deferasirox; and (b) an anti-cancer agent. In another aspect, provided herein is a combination comprising: (a) deferasirox; and (b) a PD-1/PD-L pathway inhibitor, IDO inhibitor, TDO inhibitor, CTLA-4 inhibitor, or a combination thereof. In another aspect, provided herein is a combination comprising (a) deferasirox; and (b) an antifungal agent, an antiparasitic agent, an iron chelator, a thrombopoietin (TPO) receptor agonist, or a combination thereof. In another aspect, provided herein is a combination comprising (a) deferasirox; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) deferasirox; and (b) tioconazole, liarozole, sertaconazole, econazole, sulconazole, miconazole, isoconazole, itraconazole, niclosamide, fenticonazole, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) fenticonazole; and (b) an anti-cancer agent. In another aspect, provided herein is a combination comprising: (a) fenticonazole; and (b) a PD-1/PD-L pathway inhibitor, IDO inhibitor, TDO inhibitor, CTLA-4 inhibitor, or a combination thereof. In another aspect, provided herein is a combination comprising (a) fenticonazole; and (b) an antifungal agent, an antiparasitic agent, an iron chelator, a thrombopoietin (TPO) receptor agonist, or a combination thereof. In another aspect, provided herein is a combination comprising (a) fenticonazole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) fenticonazole; and (b) tioconazole, liarozole, sertaconazole, econazole, sulconazole, miconazole, isoconazole, itraconazole, niclosamide, deferasirox, cloconazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) cloconazole; and (b) an anti-cancer agent. In another aspect, provided herein is a combination comprising: (a) cloconazole; and (b) a PD-1/PD-L pathway inhibitor, IDO inhibitor, TDO inhibitor, CTLA-4 inhibitor, or a combination thereof. In another aspect, provided herein is a combination comprising (a) cloconazole; and (b) an antifungal agent, an antiparasitic agent, an iron chelator, a thrombopoietin (TPO) receptor agonist, or a combination thereof. In another aspect, provided herein is a combination comprising (a) cloconazole; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) cloconazole; and (b) tioconazole, liarozole, sertaconazole, econazole, sulconazole, miconazole, isoconazole, itraconazole, niclosamide, deferasirox, fenticonazole, eltrombopag, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a combination comprising: (a) eltrombopag; and (b) an anti-cancer agent. In another aspect, provided herein is a combination comprising: (a) eltrombopag; and (b) a PD-1/PD-L pathway inhibitor, IDO inhibitor, TDO inhibitor, CTLA-4 inhibitor, or a combination thereof. In another aspect, provided herein is a combination comprising (a) eltrombopag; and (b) an antifungal agent, an antiparasitic agent, an iron chelator, a thrombopoietin (TPO) receptor agonist, or a combination thereof. In another aspect, provided herein is a combination comprising (a) eltrombopag; and (b) BMS-936559, MEDI4736, MPDL3280A, MSB0010718C, AMP-224, nivolumab, pembrolizumab, pidilizumab, BMS-986016, MDX1105-01, avelumab, indoximod, F001287, NLG919, INCB024360, ipilimumab, tremelimumab, antibody clone 9H10, antibody clone BNI3, Del 60, M9-14 del 55, or salts, solvates or combinations thereof. In another aspect, provided herein is a combination comprising: (a) eltrombopag; and (b) tioconazole, liarozole, sertaconazole, econazole, sulconazole, miconazole, isoconazole, itraconazole, niclosamide, deferasirox, fenticonazole, cloconazole, or salts, solvates, or combinations thereof. In some embodiments, the combination is a composition. In some embodiments, the anti-cancer agent is a component of an immunotherapy. In some embodiments, the anti-cancer agent is a component of a CAR T-cell therapy. In some embodiments, the anti-cancer agent is a component of a stem cell therapy.

In one aspect, provided herein is a method of treating cancer comprising administering to a patient in need thereof an effective amount of a compound of Formula (I), or a pharmaceutically acceptable salt or solvate thereof:

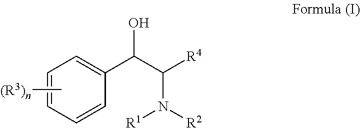

wherein
R¹ and R² are each independently selected from hydrogen or C₁-C₆ alkyl;
each R³ are independently selected from hydrogen, halogen, —OH, C₁-C₆ alkyl, or C₁-C₆ alkoxy;
R⁴ is hydrogen, halogen, or C₁-C₆ alkyl; and
n is 1, 2, 3, or 4.

In another aspect, provided herein is a method of treating cancer comprising administering to a patient in need thereof an effective amount of a compound of Formula (II), or a pharmaceutically acceptable salt or solvate thereof:

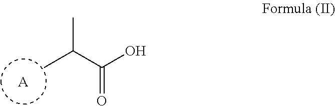

wherein
Ring A is optionally substituted aryl or optionally substituted heteroaryl.

In some embodiments, the compound of Formula (II) is of Formula (IIa):

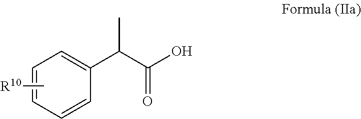

wherein
R¹⁰ is —X—R¹¹;
X is —O—, —CH₂—, —(C═O)—, or —CH═; and
R¹¹ is optionally substituted aryl, optionally substituted cycloalkyl, optionally substituted heteroaryl, or optionally substituted heterocycloalkyl.

In another aspect, provided herein is a method of treating cancer comprising administering to a patient in need thereof an effective amount of a compound of Formula (III), or a pharmaceutically acceptable salt or solvate thereof:

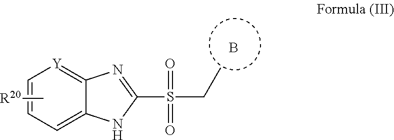

wherein
Ring B is an optionally substituted heteroaryl;
Y is —CH— or —N—; and
R²⁰ is hydrogen, halogen, C₁-C₆ alkyl, C₁-C₆ alkoxy, or C₁-C₆ haloalkoxy.

In some embodiments, the compound of Formula (III) is of Formula (IIIa):

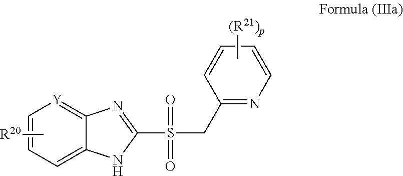

wherein
each R²¹ is independently hydrogen, C₁-C₆ alkyl, C₁-C₆ haloalkyl, C₁-C₆ alkoxy, alkoxy C₁-C₆alkoxy, or C₁-C₆ haloalkoxy; and
p is 1, 2, 3, or 4.

In another aspect, provided herein is a method of treating cancer comprising administering to a patient in need thereof an effective amount of a compound of Formula (IV), or a pharmaceutically acceptable salt or solvate thereof:

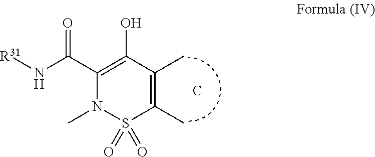

wherein
R³¹ is an optionally substituted aryl or an optionally substituted heteroaryl; and
Ring C is an optionally substituted aryl or an optionally substituted heteroaryl.

In another aspect, provided herein is a method of treating cancer comprising administering to a patient in need thereof an effective amount of a compound of Formula (V), or a pharmaceutically acceptable salt or solvate thereof:

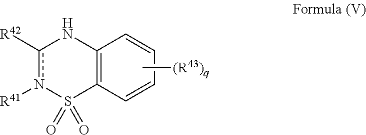

wherein [symbol] is a single bond or a double bond;
R⁴¹ is hydrogen or C₁-C₆ alkyl when [symbol] is a single bond; or
R⁴¹ is absent when [symbol] is a double bond;
R⁴² is C₁-C₆ alkyl, optionally substituted aryl, optionally substituted aralkyl, optionally substituted heteroaryl, optionally substituted heteroaralkyl, optionally substituted cycloalkyl, optionally substituted cycloalkylalkyl, or —(CH₂)—S—R⁴⁴;
each R⁴³ is independently hydrogen, halogen, C₁-C₆ alkyl, C₁-C₆ alkoxy, or —S(═O)₂NH₂;
R⁴⁴ is C₁-C₆ alkyl, C₁-C₆ haloalkyl, or optionally substituted aralkyl; and
q is 1, 2, 3, or 4.

In some embodiments, the compound of Formula (V) is of Formula (Va):

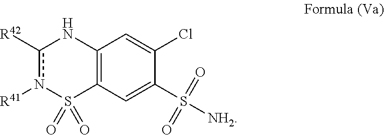

In another aspect, provided herein is a method of treating cancer comprising administering to a patient in need thereof an effective amount of a compound of Formula (VI), or a pharmaceutically acceptable salt or solvate thereof:

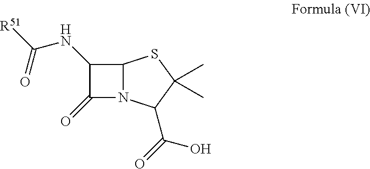

wherein
R⁵¹ is optionally substituted aryl, optionally substituted heteroaryl, or —CHR⁵²R⁵³;
R⁵² is C₁-C₆ alkyl; and
R⁵³ is optionally substituted aryl, optionally substituted heteroaryl, or optionally substituted aryloxy.

In some embodiments, the compound of Formula (VI) is of Formula (VIa):

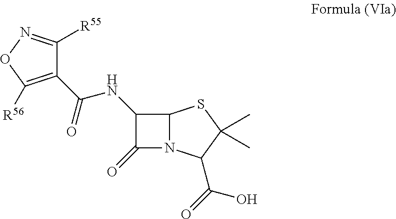

wherein
R⁵⁴ is C₁-C₆ alkyl; and
R⁵⁵ is optionally substituted aryl.

In another aspect, provided herein is a method of treating cancer comprising administering to a patient in need thereof an effective amount of a compound of Formula (VII), or a pharmaceutically acceptable salt or solvate thereof:

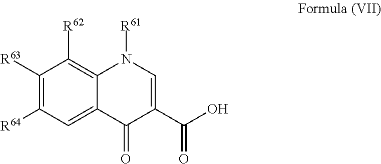

wherein
R⁶¹ is C₁-C₆ alkyl or cycloalkyl;
R⁶² is halogen, C₁-C₆ alkyl, or C₁-C₆ alkoxy; or
R⁶¹ and R⁶² taken together with the atoms to which they are attached form an optionally substituted heterocycloalkyl;
R⁶³ is optionally substituted cycloalkyl or optionally substituted heterocycloalkyl; and
R⁶⁴ is halogen, C₁-C₆ alkyl, or C₁-C₆ alkoxy.

In some embodiments, the compound of Formula (I) is a TDO inhibitor, IDO inhibitor, or both a TDO and IDO inhibitor. In some embodiments, the compound of Formula (II) is a TDO inhibitor, IDO inhibitor, or both a TDO and IDO inhibitor. In some embodiments, the compound of Formula (III) is a TDO inhibitor, IDO inhibitor, or both a TDO and IDO inhibitor. In some embodiments, the compound of Formula (IV) is a TDO inhibitor, IDO inhibitor, or both a TDO and IDO inhibitor. In some embodiments, the compound of Formula (V) is a TDO inhibitor, IDO inhibitor, or both a TDO and IDO inhibitor. In some embodiments, the compound of Formula (VI) is a TDO inhibitor, IDO inhibitor, or both a TDO and IDO inhibitor. In some embodiments, the compound of Formula (VII) is a TDO inhibitor, IDO inhibitor, or both a TDO and IDO inhibitor. In some embodiments, the compound of Formula (VIII) is a TDO inhibitor, IDO inhibitor, or both a TDO and IDO inhibitor.

In some embodiments, a method of treating cancer comprises administering to a patient in need thereof an effective amount of a compound of Formula (I), or a pharmaceutically acceptable salt or solvate thereof; an effective amount of a compound of Formula (II), or a pharmaceutically acceptable salt or solvate thereof; an effective amount of a compound of Formula (III), or a pharmaceutically acceptable salt or solvate thereof; an effective amount of a compound of Formula (IV), or a pharmaceutically acceptable salt or solvate thereof; an effective amount of a compound of Formula (V), or a pharmaceutically acceptable salt or solvate thereof; an effective amount of a compound of Formula (VI), or a pharmaceutically acceptable salt or solvate thereof; an effective amount of a compound of Formula (VII), or a pharmaceutically acceptable salt or solvate thereof; an effective amount of a compound of Formula (VIII), or a pharmaceutically acceptable salt or solvate thereof; or a combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In some embodiments, a method of treating cancer further comprises administering to the patient an effective amount of at least one PD-1/PD-L pathway inhibitor, at least one IDO inhibitor, at least one CTLA-4 inhibitor, at least one TDO inhibitor, at least one anti-cancer agent, or a combination thereof.

In one aspect of the disclosure, provided herein is a method of inhibiting TDO, IDO, or both TDO and IDO in a patient comprising administering to the patient an effective amount of an antimicrobial agent. In some embodiments, the antimicrobial agent is an antibacterial agent. In some embodiments, the antibacterial agent comprises cloxiquine, betamipronum, chloroxine, nalidixic acid, pazufloxacin, propicillin, besifloxacin, oxacillin sodium, moxifloxacin, cloxacillin, dicloxacillin, or a salt, solvate or combination thereof. In some embodiments, the antimicrobial agent is an antifungal agent. In some embodiments, the antifungal agent comprises tioconazole, liarozole, sertaconazole, econazole, sulconazole, miconazole, isoconazole, itraconazole, fenticonazole, cloconazole, acrisorcinum, climbazole, croconazole, diphenylimidazole, exalamide, lanoconazole, oxiconazole, bifonazole, thiabendazole, butoconazole, clotrimazole, ketoconazole, luliconazole, albaconazole, efinaconazole, epoxiconazole, fluconazole, isavuconazole, posaconazole, propiconazole, ravuconazole, terconazole, voriconazole, abafungin, omoconazole, phenoxyacetic acid, naftifine, tolindate, tolnaftate, butenafine, o-(5,6,7,8-tetrahydro-2-naphthyl)-6-methoxy-n-methylthio-2-pyridinecarbamate, or a salt, solvate or combination thereof. In some embodiments, the antimicrobial agent is an antiparasitic agent. In some embodiments, the antiparasitic agent comprises praziquantel, chloroquine, epsiprantel, niclosamide, hydroxychloroquin, or a salt, solvate or combination thereof. In some embodiments, the antimicrobial agent is an antiseptic. In some embodiments, the antiseptic comprises iodochlorohydroxyquinoline, euresol, acrisorcinum, benzyl peroxide, benzoyl peroxide, benzoxiquine, ethacridine lactate, or a salt, solvate or combination thereof. In some embodiments, the antimicrobial agent comprises an antiviral agent. In some embodiments, the antiviral agent comprises imiquimod, efavirenz, or a salt, solvate or combination thereof. In some embodiments, the antimicrobial agent comprises chlorquinaldol, or a salt or solvate thereof. In some embodiments, the patient has cancer and the antimicrobial agent is administered for the treatment of cancer. In some embodiments, the method further comprises administering to the patient an effective amount of an anti-cancer agent. In some embodiments, the method further comprises administering to the patient an effective amount of an additional antimicrobial agent, adrenergic agent, serotonergic agent, NSAID, sulfonamide, phosphodiesterase inhibitor, proton pump inhibitor, or a combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect of the disclosure, provided herein is a method of inhibiting TDO, IDO, or both TDO and IDO in a patient comprising administering to the patient an effective amount of an adrenergic agent. In some embodiments, the adrenergic agent comprises octopamine, ephedrine, epinephrine, levonordefrin, norfenefrine, methoxamine, mirtazapine, norpseudoephedrine, pseudophedrine, norepinephrine, adrenochrome, amezinium, clobenzorex, mirtazapine, or a salt, solvate or combination thereof. In some embodiments, the patient has cancer and the adrenergic agent is administered for the treatment of cancer. In some embodiments, the method further comprises administering to the patient an effective amount of an anti-cancer agent. In some embodiments, the method further comprises administering to the patient an effective amount of an antimicrobial agent, additional adrenergic agent, serotonergic agent, NSAID, sulfonamide, phosphodiesterase inhibitor, proton pump inhibitor, or a combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect of the disclosure, provided herein is a method of inhibiting TDO, IDO, or both TDO and IDO in a patient comprising administering to the patient an effective amount of a serotonergic agent. In some embodiments, the serotonergic agent comprises 1-tryptophan, frovatriptan succinate, ramosetron, mazindol, ondansetron, itasetron, indisetron, naratriptan, or a salt, solvate or combination thereof. In some embodiments, the patient has cancer and the serotonergic agent is administered for the treatment of cancer. In some embodiments, the method further comprises administering to the patient an effective amount of an anti-cancer agent. In some embodiments, the method further comprises administering to the patient an effective amount of an antimicrobial agent, adrenergic agent, additional serotonergic agent, NSAID, sulfonamide, phosphodiesterase inhibitor, proton pump inhibitor, or a combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect of the disclosure, provided herein is a method of inhibiting TDO, IDO, or both TDO and IDO in a patient comprising administering to the patient an effective amount of a sulfonamide. In some embodiments, the sulfonamide is a sulfonamide diuretic. In some embodiments, the sulfonamide diuretic comprises furosemide, chlorthalidone, indapamide, cyclopenthiazide, alilusem, benzthiazide, polythiazide, or a salt, solvate or combination thereof. In some embodiments, the patient has cancer and the sulfonamide is administered for the treatment of cancer. In some embodiments, the method further comprises administering to the patient an effective amount of an anti-cancer agent. In some embodiments, the method further comprises administering to the patient an effective amount of an antimicrobial agent, adrenergic agent, serotonergic agent, NSAID, additional sulfonamide, phosphodiesterase inhibitor, proton pump inhibitor, or a combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect of the disclosure, provided herein is a method of inhibiting TDO, IDO, or both TDO and IDO in a patient comprising administering to the patient an effective amount of an NSAID. In some embodiments, the NSAID comprises glucosamine, nabumetone, fenoprofen, loxoprofen, diflunisal, ketoprofen, dexketoprofen, fenbufen, nepafenac, tolmetin, pelubiprofen, tiaprofenic acid, suprofen, metiazinic acid, carprofen, clidanac, etodolac, zaltoprofen, nifenazone, rofecoxib, protizinic acid, methyl salicylate, piroxicam, meloxicam, diacerein, lornoxicam, glafenine, or a salt, solvate, or combination thereof. In some embodiments, the patient has cancer and the NSAID is administered for the treatment of cancer. In some embodiments, the method further comprises administering to the patient an effective amount of an anti-cancer agent. In some embodiments, the method further comprises administering to the patient an effective amount of an antimicrobial agent, adrenergic agent, serotonergic agent, additional NSAID, sulfonamide, phosphodiesterase inhibitor, proton pump inhibitor, or a combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect of the disclosure, provided herein is a method of inhibiting TDO, IDO, or both TDO and IDO in a patient comprising administering to the patient an effective amount of a phosphodiesterase inhibitor. In some embodiments, the phosphodiesterase inhibitor comprises theophylline, ibudilast, anagrelide, doxofylline, rolipram, cilomilast, vinpocetine, or a salt, solvate or combination thereof. In some embodiments, the patient has cancer and the phosphodiesterase inhibitor is administered for the treatment of cancer. In some embodiments, the method further comprises administering to the patient an effective amount of an anti-cancer agent. In some embodiments, the method further comprises administering to the patient an effective amount of an antimicrobial agent, adrenergic agent, serotonergic agent, NSAID, sulfonamide, additional phosphodiesterase inhibitor, proton pump inhibitor, or a combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect of the disclosure, provided herein is a method of inhibiting TDO, IDO, or both TDO and IDO in a patient comprising administering to the patient an effective amount of a proton pump inhibitor. In some embodiments, the proton pump inhibitor comprises esomeprazole, omeprazole, tenatoprazole, rabeprazole, lansoprazole, pantoprazole, or a salt, solvate or combination thereof. In some embodiments, the patient has cancer and the proton pump inhibitor is administered for the treatment of cancer. In some embodiments, the method further comprises administering to the patient an effective amount of an anti-cancer agent. In some embodiments, the method further comprises administering to the patient an effective amount of an antimicrobial agent, adrenergic agent, serotonergic agent, NSAID, sulfonamide, phosphodiesterase inhibitor, additional proton pump inhibitor, or a combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In one aspect, provided herein is a method of inhibiting TDO in a patient comprising administering to the patient an effective amount of a composition comprising econazole, sulconazole, isoconazole, miconazole, sertaconazole, tioconazole, fenticonazole, liarozole, cloconazole, itraconazole, niclosamide, deferasirox, eltrombopag, allopurinol, indium in-111 oxyquinoline, coumarin, lutine, phenoxyacetic acid, mesalazine, euresol, norpseudoephedrine, octopamine, norfenefrine, allantoin, sodium phenylbutyrate, (r)-3-(methoxymethyl)-2,3-dihydro-1h-pyrrolo[2,3-b]pyridine, 2-(dimethylamino)-1-phenylethanol, edrophonium, ephedrine, pseudophedrine, pyridoxine, chlorzoxazone, norepinephrine, edaravone, cantabiline, methoxycinnamate, cloxiquine, glucose, theophylline, glucosamine, adrenochrome, fludeoxyglucose, epinephrine, levonordefrin, dibenzothiophene, betamipronum, sodium ferulic, caffeine, acrisorcinum, tacrine, amezinium, 1-tryptophan, trapidil, alphalipoic acid, lipoic acid, caldibasic phosphate, methoxamine, benzyl nicotinate, chloroxine, benzyl peroxide, methoxsalen, santoquin, n-acetylmannosamine, (1)-isomer, hydrocotarnine, guaiac, dithranol, hydroxyethyltheophylline, chlorquinaldol, nabumetone, ibudilast, diazoxide, acitazanolast, nalidixic acid, melatonin, eptazocine, 4-methoxy-2-(5-methoxy-3-methyl-1h-pyrazol-1-yl)-6-methyl-pyrimidine, rufinamide, 1,8-dihydroxyanthraquinone, benzyl cinnamate, (z)-3-((3,5-dimethyl-1h-pyrrol-2-yl)methylene)indolin-2-one, felbinac ethyl ester, imiquimod, methocarbamol, fenoprofen, benzoyl peroxide, (e)-2-(isoxazol-3(2h)-ylidene)-2,6,7,9-tetrahydroimidazo[4,5-d]pyrano[4,3-b]pyridine, huperzine a, frovatriptan succinate, loxoprofen, chlorphenesin carbamate, tolperisone, erdosteine, diflunisal, benzoxiquine, 5-(dimethylamino)naphthalene-2-sulfonic acid, 2-(4-morpholino)thiobenzothiazole, menadione, menadione sodium bisulfite, ketoprofen, dexketoprofen, fenbufen, nepafenac, ethacridine lactate, tolmetin, petoxil, anagrelide, pelubiprofen, tiaprofenic acid, suprofen, 1-(3,4-dihydro-1h-pyrido[3,4-b]indol-2(9h)-yl)-3-mercaptopropan-1-one, clobenzorex, hexylcaine, n-cyclohexyl-2-benzothiazylsulfenamide, doxofylline, mirtazapine, emodin, metiazinic acid, carprofen, perisoxalum, zelandopam, rolipram, cyclandelate, clidanac, venlafaxine, phenprocoumon, esonarimod, ramosetron, nsc-4911, mazindol, etodolac, naftifine, lobenzarit, ondansetron, leucogen, zaltoprofen, tolindate, ampholyt g, itasetron, chlorambucil, zaleplon, pirenoxine, tolnaftate, nifenazone, cycotiamine, d-panthenol, trenbolone acetate, praziquantel, rofecoxib, protizinic acid, indisetron, efavirenz, nateglinide, choline fenofibrate, pazufloxacin, butenafine, (r)-(5-(3-aminophenyl)-1h-pyrrolo[2,3-b]pyridin-3-yl)(5-methyl-4,5-dihydroisoxazol-3-yl)methanone, methyl salicylate, chloroquine, glabridin, epsiprantel, cyclic adenosine monophosphate, o-(5,6,7,8-tetrahydro-2-naphthyl)-6-methoxy-n-methylthio-2-pyridinecarbamate, furosemide, piroxicam, mitomycin, berberine, naratriptan, hydroxychloroquine, chlorthalidone, cilomilast, esomeprazole, omeprazole, tenatoprazole, meloxicam, vinpocetine, repirinast, rabeprazole, indapamide, diacerein, blonanserin, lansoprazole, lornoxicam, glafenine, propicillin, cyclopenthiazide, droperidol, benperidol, pantoprazole, benzlhydrochlorothiazide, cyclothiazide, abiraterone acetate, alilusem, besifloxacin, (2s,3r,4r,5r,6r)-3-((3s,4s,5r,6s)-6-carboxy-3,4-dihydroxy-5-methoxytetrahydro-2h-pyran-2-yloxy)-4,5-dihydroxy-6-methoxytetrahydro-2h-pyran-2-carboxylic acid, sunitinib, oxacillin sodium, moxifloxacin, acetophenazine, noscapine, carfenazine, domperidone, benzthiazide, cloxacillin, polythiazide, pimozide, dicloxacillin, thiabendazole, zonisamide, bendazac, alosetron, dolasetron, timiperone, iodochlorohydroxyquinoline, bifonazole, butoconazole, clotrimazole, ketoconazole, luliconazole, albaconazole, efinaconazole, epoxiconazole, fluconazole, isavuconazole, posaconazole, propiconazole, ravuconazoie, terconazole, voriconazole, abafungin, omoconazole, lanoconazole, nimustine, climbazole, nedocromil, pemirolast, granisetron, methysergide, sorbinil, tolazoline, metronidazole, dexmedetomidine, ornidazole, diphenylimidazole, tinidazole, sulfamethoxazole, pyrrolnitrin, benznidazole, nevirapine, rizatriptan, mebhydroline, niturtatel, zolmitriptan, nitazoxanide, alcaftadine, sulfaphenazole, fexbuxostat, protirelin, bromhexine, rivaroxaban, letrozole, hydrobenzole, ozagrel, procainamide, sulfisoxazole, sulfamethizole, chlorpropamide, anastrozole, flumazenil, sulfadimethoxine, succinylsulfathiazole, At-7519, rebamipide, taltirelin, sitagliptin, ezetimibe, bicalutamide, or a salt, solvate or combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In some embodiments, the patient has cancer and the composition is administered for the treatment of cancer. In some embodiments, the method further comprises administering to the patient an effective amount of an anti-cancer agent. In some embodiments, the method further comprises administering to the patient an effective amount of an antimicrobial agent, adrenergic agent, serotonergic agent, NSAID, sulfonamide, phosphodiesterase inhibitor, proton pump inhibitor, or a combination thereof. In some embodiments, the method further comprises administering to the patient a CTLA-4 inhibitor. In some embodiments, the CTLA-4 inhibitor comprises ipilimumab, tremelimumab, antibody clone 9H10, antibody clone BNI3, Del 60, M9-14 del 55, or a combination thereof. In some embodiments, the method further comprises administering to the patient an effective amount of a PD-1/PD-L pathway inhibitor. In some embodiments, the PD-1/PD-L pathway inhibitor comprises BMS-936559, MEDI4736, MPDL3280A, MSB0010718C, AMP-224, nivolumab, pembrolizumab, pidilizumab, BMS-986016, MDX1105-01, avelumab, or a salt, solvate, or combination thereof. In some embodiments, the method further comprises administering to the patient an effective amount of an IDO pathway inhibitor. In some embodiments, the IDO inhibitor comprises indoximod (D-1MT), F001287, NLG919, INCB024360, or a salt, solvate or combination thereof.

In another aspect, provided herein is a method of inhibiting IDO in a patient comprising administering to the patient an effective amount of a composition comprising econazole, sulconazole, isoconazole, miconazole, sertaconazole, tioconazole, fenticonazole, liarozole, cloconazole, itraconazole, niclosamide, deferasirox, eltrombopag, allopurinol, indium in-111 oxyquinoline, coumarin, lutine, phenoxyacetic acid, mesalazine, euresol, norpseudoephedrine, octopamine, norfenefrine, allantoin, sodium phenylbutyrate, (r)-3-(methoxymethyl)-2,3-dihydro-1h-pyrrolo[2,3-b]pyridine, 2-(dimethylamino)-1-phenylethanol, edrophonium, ephedrine, pseudophedrine, pyridoxine, chlorzoxazone, norepinephrine, edaravone, cantabiline, methoxycinnamate, cloxiquine, glucose, theophylline, glucosamine, adrenochrome, fludeoxyglucose, epinephrine, levonordefrin, dibenzothiophene, betamipronum, sodium ferulic, caffeine, acrisorcinum, tacrine, amezinium, 1-tryptophan, trapidil, alphalipoic acid, lipoic acid, caldibasic phosphate, methoxamine, benzyl nicotinate, chloroxine, benzyl peroxide, methoxsalen, santoquin, n-acetylmannosamine, (1)-isomer, hydrocotarnine, guaiac, dithranol, hydroxyethyltheophylline, chlorquinaldol, nabumetone, ibudilast, diazoxide, acitazanolast, nalidixic acid, melatonin, eptazocine, 4-methoxy-2-(5-methoxy-3-methyl-1h-pyrazol-1-yl)-6-methyl-pyrimidine, rufinamide, 1,8-dihydroxyanthraquinone, benzyl cinnamate, (z)-3-((3,5-dimethyl-1h-pyrrol-2-yl)methylene)indolin-2-one, felbinac ethyl ester, imiquimod, methocarbamol, fenoprofen, benzoyl peroxide, (e)-2-(isoxazol-3(2h)-ylidene)-2,6,7,9-tetrahydroimidazo[4,5-d]pyrano[4,3-b]pyridine, huperzine a, frovatriptan succinate, loxoprofen, chlorphenesin carbamate, tolperisone, erdosteine, diflunisal, benzoxiquine, 5-(dimethylamino)naphthalene-2-sulfonic acid, 2-(4-morpholino)thiobenzothiazole, menadione, menadione sodium bisulfite, ketoprofen, dexketoprofen, fenbufen, nepafenac, ethacridine lactate, tolmetin, petoxil, anagrelide, pelubiprofen, tiaprofenic acid, suprofen, 1-(3,4-dihydro-1h-pyrido[3,4-b]indol-2(9h)-yl)-3-mercaptopropan-1-one, clobenzorex, hexylcaine, n-cyclohexyl-2-benzothiazylsulfenamide, doxofylline, mirtazapine, emodin, metiazinic acid, carprofen, perisoxalum, zelandopam, rolipram, cyclandelate, clidanac, venlafaxine, phenprocoumon, esonarimod, ramosetron, nsc-4911, mazindol, etodolac, naftifine, lobenzarit, ondansetron, leucogen, zaltoprofen, tolindate, ampholyt g, itasetron, chlorambucil, zaleplon, pirenoxine, tolnaftate, nifenazone, cycotiamine, d-panthenol, trenbolone acetate, praziquantel, rofecoxib, protizinic acid, indisetron, efavirenz, nateglinide, choline fenofibrate, pazufloxacin, butenafine, (r)-(5-(3-aminophenyl)-1h-pyrrolo[2,3-b]pyridin-3-yl)(5-methyl-4,5-dihydroisoxazol-3-yl)methanone, methyl salicylate, chloroquine, glabridin, epsiprantel, cyclic adenosine monophosphate, o-(5,6,7,8-tetrahydro-2-naphthyl)-6-methoxy-n-methylthio-2-pyridinecarbamate, furosemide, piroxicam, mitomycin, berberine, naratriptan, hydroxychloroquine, chlorthalidone, cilomilast, esomeprazole, omeprazole, tenatoprazole, meloxicam, vinpocetine, repirinast, rabeprazole, indapamide, diacerein, blonanserin, lansoprazole, lornoxicam, glafenine, propicillin, cyclopenthiazide, droperidol, benperidol, pantoprazole, benzylhydrochlorothiazide, cyclothiazide, abiraterone acetate, alilusem, besifloxacin, (2s,3r,4r,5r,6r)-3-((3s,4s,5r,6s)-6-carboxy-3,4-dihydroxy-5-methoxytetrahydro-2h-pyran-2-yloxy)-4,5-dihydroxy-6-methoxytetrahydro-2h-pyran-2-carboxylic acid, sunitinib, oxacillin sodium, moxifloxacin, acetophenazine, noscapine, carfenazine, domperidone, benzthiazide, cloxacillin, polythiazide, pimozide, dicloxacillin, thiabendazole, zonisamide, bendazac, alosetron, dolasetron, timiperone, iodochlorohydroxyquinoline, bifonazole, butoconazole, clotrimazole, ketoconazole, luliconazole, albaconazole, efinaconazole, epoxiconazole, fluconazole, isavuconazole, posaconazole, propiconazole, ravuconazole, terconazole, voriconazole, abafungin, omoconazole, lanoconazole, nimustine, climbazole, nedocromil, pemirolast, granisetron, methysergide, sorbinil, tolazoline, metronidazole, dexmedetomidine, ornidazole, diphenylimidazole, tinidazole, sulfamethoxazole, pyrrolnitrin, benznidazole, nevirapine, rizatriptan, mebhydroline, niturtatel, zolmitriptan, nitazoxanide, alcaftadine, sulfaphenazole, fexbuxostat, protirelin, bromhexine, rivaroxaban, letrozole, hydrobenzole, ozagrel, procainamide, sulfisoxazole, sulfamethizole, chlorpropamide, anastrozole, flumazenil, sulfadimethoxine, succinylsulfathiazole, At-7519, rebamipide, taltirelin, sitagliptin, ezetimibe, bicalutamide, or a salt, solvate or combination thereof. In some embodiments, the method further comprises treating the individual with an immunotherapy. In some embodiments, the method further comprises treating the individual with a chimeric antigen receptor (CAR) T-cell therapy. In some embodiments, the method further comprises treating the individual with a stem cell therapy.

In some embodiments, the patient has cancer and the composition is administered for the treatment of cancer. In some embodiments, the method further comprises administering to the patient an effective amount of an anti-cancer agent. In some embodiments, the method further comprises administering to the patient an effective amount of an antimicrobial agent, adrenergic agent, serotonergic agent, NSAID, sulfonamide, phosphodiesterase inhibitor, proton pump inhibitor, or a combination thereof. In some embodiments, the method further comprises administering to the patient a CTLA-4 inhibitor. In some embodiments, the CTLA-4 inhibitor comprises ipilimumab, tremelimumab, antibody clone 9H10, antibody clone BNI3, Del 60, M9-14 del 55, or a combination thereof. In some embodiments, the method further comprises administering to the patient an effective amount of a PD-1/PD-L pathway inhibitor. In some embodiments, the PD-1/PD-L pathway inhibitor comprises BMS-936559, MEDI4736, MPDL3280A, MSB0010718C, AMP-224, nivolumab, pembrolizumab, pidilizumab, BMS-986016, MDX1105-01, avelumab, or a salt, solvate, or combination thereof. In some embodiments, the method further comprises administering to the patient an effective amount of an IDO pathway inhibitor. In some embodiments, the IDO inhibitor comprises indoximod (D-1MT), F001287, NLG919, INCB024360, or a salt, solvate or combination thereof.

### BRIEF DESCRIPTION OF THE FIGURES

The novel and inventive features of the subject matter described herein are set forth with particularity in the appended claims. A better understanding of the features and advantages of the present invention will be obtained by reference to the following detailed description that sets forth illustrative embodiments, in which the principles of the invention are utilized, and accompanying drawings of which:

FIG. 1 shows plots of viable CT26 murine colon cancer cells as a percent of total cells versus concentration of TDO/IDO inhibitor test compounds. The graph of panel A shows the effect of test compounds tioconazole, niclosamide, eltrombopag, deferasirox, and control compound 1MT on CT26 viability. The graph of panel B shows the effect of test compounds sulconazole, cloconazole, miconazole, and control compound 1MT on C26 viability.

FIG. 2 shows plots of percent viable CD4+ cells versus concentration of TDO/IDO inhibitor test compounds. The graph of panel A shows the effect of test compounds tioconazole, niclosamide, eltrombopag, deferasirox, and control compound 1MT on CD4+ viability. The graph of panel B shows the effect of test compounds sulconazole, cloconazole, miconazole, and control compound 1MT on CD430 viability.

FIG. 3 shows plots of percent viable CD69+CD4+ cells versus concentration of TDO/IDO inhibitor test compounds. The graph of panel A shows the effect of test compounds tioconazole, niclosamide, eltrombopag, deferasirox, and control compound 1MT on CD4+ viability. The graph of panel B shows the effect of test compounds sulconazole, cloconazole, miconazole, and control compound 1MT on CD430 viability.

FIG. 4 shows plots of percent viable CD71+CD4+ cells versus concentration of TDO/IDO inhibitor test compounds. The graph of panel A shows the effect of test compounds tioconazole, niclosamide, eltrombopag, deferasirox, and control compound 1MT on CD4+ viability. The graph of panel B shows the effect of test compounds sulconazole, cloconazole, miconazole, and control compound 1MT on CD430 viability.

FIG. 5 shows plots of percent viable CD8+ cells versus concentration of TDO/IDO inhibitor test compounds. The graph of panel A shows the effect of test compounds tioconazole, niclosamide, eltrombopag, deferasirox, and control compound 1MT on CD8+ viability. The graph of panel B shows the effect of test compounds sulconazole, cloconazole, miconazole, and control compound 1MT on CD830 viability.

FIG. 6 shows plots of percent viable CD69+CD8+ cells versus concentration of TDO/IDO inhibitor test compounds. The graph of panel A shows the effect of test compounds tioconazole, niclosamide, eltrombopag, deferasirox, and control compound 1MT on CD8+ viability. The graph of panel B shows the effect of test compounds sulconazole, cloconazole, miconazole, and control compound 1MT on CD830 viability.

FIG. 7 shows plots of percent viable CD71+CD8+ cells versus concentration of TDO/IDO inhibitor test compounds. The graph of panel A shows the effect of test compounds tioconazole, niclosamide, eltrombopag, deferasirox, and control compound 1MT on CD8+ viability. The graph of panel B shows the effect of test compounds sulconazole, cloconazole, miconazole, and control compound 1MT on CD830 viability.

### DETAILED DESCRIPTION OF THE INVENTION

Immunotherapeutic approaches for treating cancer often comprise the manipulation of the immune system's T-cell response by disrupting mechanisms by which tumor cells evade the immune system. One method of reducing tumor cell evasion involves the use of immune checkpoint inhibitors to potentiate the antitumor T-cell response. An exemplary immune checkpoint inhibitor for the treatment of cancer is indoleamine 2,3-dioxygenase (IDO). The enzymatic activity of IDO is similar to that of tryptophan 2,3-dioxygenase (TDO) and studies have implicated TDO in the development of cancer in a similar manner to IDO. Disclosed herein, in various aspects, are compositions comprising TDO and/or IDO inhibitors and optionally one or more additional agents, such as one or more additional immune checkpoint inhibitors, useful for the treatment of cancer. In various aspects, a TDO inhibitor is provided that also functions as an inhibitor of IDO, and vice versa. In some embodiments, the TDO and/or IDO inhibitor compositions provided herein are useful for the treatment of non-cancerous diseases or conditions, for example, infectious diseases. In some embodiments, the TDO and/or IDO inhibitors provided herein are useful in immunotherapy, stem cell therapy, and/or CAR T-cell therapy.

In the following description, certain specific details are set forth in order to provide a thorough understanding of various embodiments. However, one skilled in the art will understand that the embodiments provided may be practiced without these details. In some instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The terms below, as used herein, have the following meanings, unless indicated otherwise:

“Oxo” refers to the ═O substituent.

“Thioxo” refers to the ═S substituent.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl, ethyl, s-butyl, or 1-ethyl-propyl. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkylene” refers to an alkyl radical as described above where one or more carbon atoms of the alkyl is replaced with a O, N, or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl and naphthalenyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

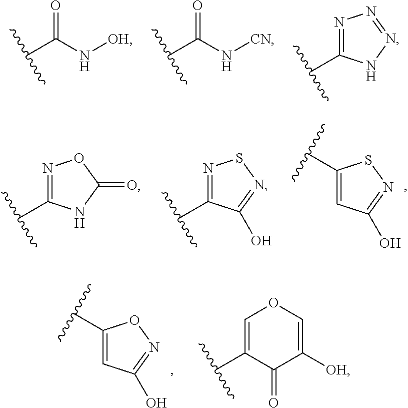

and the like.

“Cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloakyls having from three to ten carbon atoms, from three to eight carbon atoms, from three to six carbon atoms, or from three to five carbon atoms. Monocyclic cycicoalkyl radicals include, for example, cyclopropyl, cyclobutyl, cyclopentyl, cyclohexyl, cycloheptyl, and cyclooctyl. In some embodiments, the monocyclic cycicoalkyl is cyclopropyl, cyclobutyl, cyclopentyl, or cyclohexyl. Polycyclic radicals include, for example, adamantyl, norbornyl, decalinyl, and 3,4-dihydronaphthalen-1(2H)-one. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro, or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 13 carbon atoms and from one to 6 heteroatoms selected from the group consisting of nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. The term heterocycloalkyl also includes all ring forms of carbohydrates, including but not limited to monosaccharides, disaccharides, and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen, and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl, or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆ alkylalkyne, halogen, acyl, acyloxy, —CO₂H, —CO₂ alkyl, nitro, and amino, including mono- and di-substituted amino groups (e.g. —NH₂, —NHR, —N(R)₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

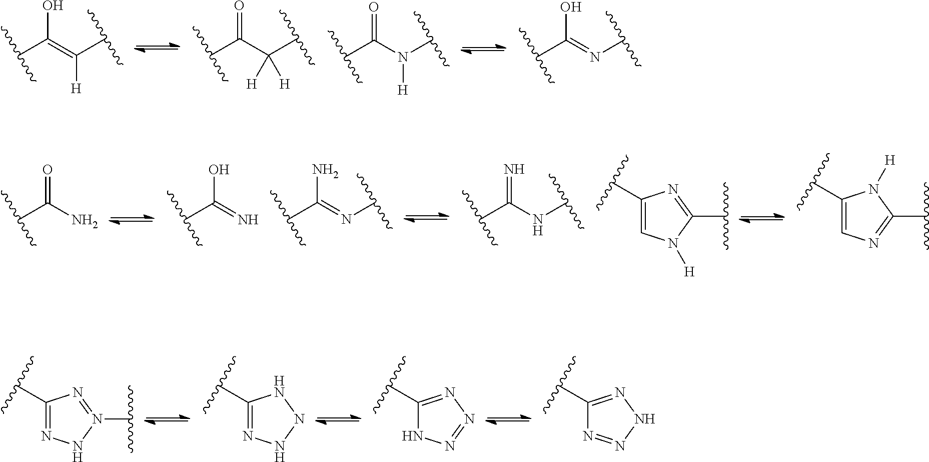

In one aspect, the compounds disclosed herein possess one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In some embodiments, sites on the aromatic ring portion of compounds described herein are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen or an alkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

“Pharmaceutically acceptable” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound of Formula (I), (II), (III), (IV), (V), (VI), (VII), (VIII), (IX) with acids. Pharmaceutically acceptable salts are also obtained by reacting a compound of Formula (I), (II), (III), (IV), (V), (VI), (VII), (VIII), (IX) with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Compounds and Compositions

In one aspect, provided herein are compounds and compositions comprising one or more TDO inhibitors, IDO inhibitors, and/or inhibitors of both TDO and IDO. In some embodiments, a TDO inhibitor is also an inhibitor of IDO, and vice versa. In some cases wherein a TDO inhibitor is also an inhibitor of IDO, the TDO inhibitory activity is greater than the IDO inhibitory activity. In some cases wherein a TDO inhibitor is also an inhibitor of IDO, the IDO inhibitory activity is greater than the TDO inhibitory activity. In some cases wherein an IDO inhibitor is also an inhibitor of TDO, the IDO inhibitory activity is greater than the TDO inhibitory activity. In some cases wherein an IDO inhibitor is also an inhibitor of TDO, the TDO inhibitory activity is greater than the IDO inhibitory activity. In some cases wherein a TDO inhibitor is also an inhibitor of IDO, the TDO inhibitory activity is comparable to that of the IDO inhibitory activity. For example, comparable includes activity values as measured by any in vitro and/or in vitro method(s) known in the art that are within 30%, 25%, 20%, 15%, 10%, 5%, 1% or less of one another. In some embodiments, a TDO inhibitor provided herein has little or no measurable activity for inhibiting TDO and has measurable activity for inhibiting IDO. In some embodiments, an IDO inhibitor provided herein has little or no measurable activity for inhibiting IDO and has measurable activity for inhibiting TDO.

In some embodiments, inhibitors of TDO, IDO, or both TDO and IDO are referred to as TDO/IDO inhibitors. In some cases, a TDO/IDO inhibitor has in vitro and/or in vivo activity for the inhibition of TDO. In some cases, a TDO/IDO inhibitor has in vitro and/or in vivo activity for the inhibition of IDO. In some cases, a TDO/IDO inhibitor has in vitro and/or activity for the inhibition of TDO and IDO. In some cases, a TDO/IDO inhibitor has in vitro and/or in vivo activity for the inhibition of TDO and little or no measurable activity for the inhibition of IDO. In some cases, a TDO/IDO inhibitor has in vitro and/or in vivo activity for the inhibition of IDO and little or no measurable activity for the inhibition of TDO.

In some embodiments, a TDO/IDO inhibitor is a small molecule. In some embodiments, a TDO/IDO inhibitor is a peptide. In some cases, a TDO/IDO inhibitor is not an immunoglobulin or immunoglobulin derived protein. In some embodiments, a TDO/IDO inhibitor directly or indirectly inhibits TDO, IDO, or both TDO and IDO. In some embodiments, a TDO/IDO inhibitor refers to a compound that targets TDO, IDO, or both TDO and IDO. In some instances, a TDO/IDO inhibitor targets one or more proteins in a signaling cascade comprising TDO, IDO, or both TDO and IDO. In some instances, a TDO/IDO inhibitor inhibits TDO, IDO, or both TDO and IDO expression. In some embodiments, a composition comprising one or more TDO/IDO inhibitors provided herein further comprises or is combined with one or more additional active agents. In some cases, an additional active agent is a PD-1 inhibitor. PD-1 inhibitors include any inhibitors of the PD-1/PD-L pathway. In some instances, an additional active agent is an IDO inhibitor or an additional IDO inhibitor. In some instances, an additional active agent is a CTLA-4 inhibitor. In some instances, an additional active agent is an IDO inhibitor or another TDO inhibitor. In some cases, an additional active agent is an anti-cancer agent. In some cases, the anti-cancer agent is a PD-1 inhibitor. In some cases, the anti-cancer agent is an IDO inhibitor. In some cases, the anti-cancer agent is a CTLA-4 inhibitor. In some cases, the anti-cancer agent is a TDO inhibitor. In various embodiments, an anti-cancer agent is an agent that modulates the immune system to treat cancer. For example, in order exploit the intrinsic potential of the immune system to recognize and/or eliminate tumor cells.

Non-limiting examples of TDO inhibitors, IDO inhibitors, or inhibitors of both TDO and IDO are provided in Table 1 and include salts and solvates of the compounds of Table 1. Additional examples of TDO inhibitors, IDO inhibitors, or inhibitors of both TDO and IDO include, without limitation, indoximod (D-1MT), F001287, NLG919, INCB024360, and salts, solvates and combinations thereof.

The compositions and TDO/IDO inhibitors provided herein, in various aspects, are useful for the treatment of cancer. In some embodiments, the TDO/IDO inhibitors provided herein are useful in immunotherapy, stem cell therapy, and/or CAR T-cell therapy. In some embodiments, a composition comprising one or more TDO/IDO inhibitors described herein is useful for the treatment of cancer. In some embodiments, provided herein are methods of treating cancer comprising administering one or more TDO/IDO inhibitors provided herein, for example, one or more compounds of Table 1, or salts or solvates of one or more compounds of Table 1. In some cases, a composition for treating cancer comprises 2, 3, 4, or 5 TDO/IDO inhibitors. For example, a composition comprises 1, 2, 3, 4, or 5 TDO/IDO inhibitors from Table 1, or salts or solvates thereof, and optionally 1 or more additional TDO/IDO inhibitors, such as indoximod (D-1MT), F001287, NLG919, INCB024360, or salts or solvates thereof.

TABLE 1
TDO/IDO Inhibitors.
TDO/IDO Inhibitor Compound Name

(2s,3r,4r,5r,6r)-3-((3s,4s,5r,6s)-6-carboxy-3,4-dihydroxy-5-
methoxytetrahydro-2h-pyran-2-yloxy)-4,5-dihydroxy-6-
methoxytetrahydro-2h-pyran-2-carboxylic acid
(e)-2-(isoxazol-3(2h)-ylidene)-2,6,7,9-tetrahydroimidazo[4,5-d]pyrano[4,3-
b]pyridine
(r)-(5-(3-aminophenyl)-1h-pyrrolo[2,3-b]pyridin-3-yl)(5-methyl-4,5-
dihydroisoxazol-3-yl)methanone
(r)-3-(methoxymethyl)-2,3-dihydro-1h-pyrrolo[2,3-b]pyridine
(z)-3-((3,5-dimethyl-1h-pyrrol-2-yl)methylene)indolin-2-one
1-(3,4-dihydro-1h-pyrido[3,4-b]indol-2(9h)-yl)-3-mercaptopropan-1-one
1,8-dihydroxyanthraquinone
2-(4-morpholino)thiobenzothiazole
2-(dimethylamino)-1-phenylethanol
4-methoxy-2-(5-methoxy-3-methyl-1h-pyrazol-1-yl)-6-methyl- pyrimidine
5-(dimethylamino)naphthalene-2-sulfonic acid
abafungin
abiraterone acetate
acetophenazine
acitazanolast
acrisorcinum
adrenochrome
albaconazole
alcaftadine
alilusem
allantoin
allopurinol
alosetron
alphalipoic acid
amezinium
ampholyt g
anagrelide
anastrozole
at-7519
bendazac
benperidol
benznidazole
benzoxiquine
benzoyl peroxide
benzthiazide
benzyl cinnamate
benzyl nicotinate
benzyl peroxide
benzylhydrochlorothiazide
berberine
besifloxacin
betamipronum
bicalutamide
bifonazole
blonanserin
bromhexine
butenafine
butoconazole
caffeine
caldibasic phosphate
cantabiline
carfenazine
carprofen
chlorambucil
chloroquine
chloroxine
chlorphenesin carbamate
chlorpropamide
chlorquinaldol
chlorthalidone
chlorzoxazone
choline fenofibrate
cilomilast
clidanac
climbazole
clobenzorex
cloconazole
clotrimazole
cloxacillin
cloxiquine
coumarin
cyclandelate
cyclic adenosine monophosphate
cyclopenthiazide
cyclothiazide
cycotiamine
deferasirox
dexketoprofen
dexmedetomidine
diacerein
diazoxide
dibenzothiophene
dicloxacillin
diflunisal
diphenylimidazole
dithranol
dolasetron
domperidone
doxofylline
d-panthenol
droperidol
econazole
edaravone
edrophonium
efavirenz
efinaconazole
eltrombopag
emodin
ephedrine
epinephrine
epoxiconazole
epsiprantel
eptazocine
erdosteine
esomeprazole
esonarimod
ethacridine lactate
etodolac
euresol
ezetimibe
felbinac ethyl ester
fenbufen
fenoprofen
fenticonazole
fexbuxostat
fluconazole
fludeoxyglucose
flumazenil
frovatriptan succinate
furosemide
glabridin
glafenine
glucosamine
glucose
granisetron
guaiac
hexylcaine
huperzine a
hydrobenzole
hydrocotarnine
hydroxychloroquine
hydroxyethyltheophylline
ibudilast
imiquimod
indapamide
indisetron
indium in-111 oxyquinoline
iodochlorohydroxyquinoline
isavuconazole
isoconazole
itasetron
itraconazole
ketoconazole
ketoprofen
lanoconazole
lansoprazole
letrozole
leucogen
levonordefrin
liarozole
lipoic acid
lobenzarit
lornoxicam
loxoprofen
1-tryptophan
luliconazole
lutine
mazindol
mebhydroline
melatonin
meloxicam
menadione
menadione sodium bisulfite
mesalazine
methocarbamol
methoxamine
methoxsalen
methoxycinnamate
methyl salicylate
methysergide
metiazinic acid
metronidazole
miconazole
mirtazapine
mitomycin
moxifloxacin
nabumetone
n-acetylmannosamine (1)-isomer
naftifine
nalidixic acid
naratriptan
nateglinide
n-cyclohexyl-2-benzothiazylsulfenamide
nedocromil
nepafenac
nevirapine
niclosamide
nifenazone
nimustine
nitazoxanide
niturtatel
norepinephrine
norfenefrine
norpseudoephedrine
noscapine
nsc-4911
o-(5,6,7,8-tetrahydro-2-naphthyl)-6-methoxy-n-methylthio-2-
pyridinecarbamate
octopamine
omeprazole
omoconazole
ondansetron
ornidazole
oxacillin sodium
oxiconazole
ozagrel
pantoprazole
pazufloxacin
pelubiprofen
pemirolast
perisoxalum
petoxil
phenoxyacetic acid
phenprocoumon
pimozide
pirenoxine
piroxicam
polythiazide
posaconazole
praziquantel
procainamide
propicillin
propiconazole
protirelin
protizinic acid
pseudophedrine
pyridoxine
pyrrolnitrin
rabeprazole
ramosetron
ravuconazole
rebamipide
repirinast
rivaroxaban
rizatriptan
rofecoxib
rolipram
rufinamide
santoquin
sertaconazole
sitagliptin
sodium ferulic
sodium phenylbutyrate
sorbinil
succinylsulfathiazole
sulconazole
sulfadimethoxine
sulfamethizole
sulfamethoxazole
sulfaphenazole
sulfisoxazole
sunitinib
suprofen
tacrine
taltirelin
tenatoprazole
terconazole
theophylline
thiabendazole
tiaprofenic acid
timiperone
tinidazole
tioconazole
tolazoline
tolindate
tolmetin
tolnaftate
tolperisone
trapidil
trenbolone acetate
venlafaxine
vinpocetine
voriconazole
zaleplon
zaltoprofen
zelandopam
zolmitriptan
zonisamide

In various aspects, compositions comprising one or more TDO/IDO inhibitors (e.g., 1, 2, 3, 4, 5, or more) further comprise and/or are administered with one or more PD-1 inhibitors (e.g., 1, 2, 3, 4, 5, or more). In some embodiments, the compositions comprising one or more TDO/IDO inhibitors and one or more PD-1 inhibitors are useful for the treatment of cancer. In some embodiments, the compositions comprising one or more TDO/IDO inhibitors administered with one or more PD-1 inhibitors are useful for the treatment of cancer. As used herein, PD-1 inhibitors include inhibitors of the PD-1 ligand pathway. Non-limiting examples of PD-1 inhibitors include BMS-936559, MEDI4736, MPDL3280A, MSB0010718C, AMP-224, nivolumab, pembrolizumab, pidilizumab, BMS-986016, MDX1105-01, avelumab, and salts and solvates thereof. Additional examples of PD-1 inhibitors include, without limitation, the compounds listed in Table 2, and salts, solvates and combinations thereof. In some instances, the compositions comprising one or more TDO/IDO inhibitors and one or more PD-1 inhibitors further comprise one or more additional active agents. In some instances, the compositions comprising one or more TDO/IDO inhibitors administered with one or more PD-1 inhibitors are further administered with one or more additional active agents. Additional active agents include, without limitation, anti-cancer agents, IDO inhibitors (e.g., Table 1), TDO inhibitors (e.g., Table 1), CTLA-4 inhibitors (e.g., Table 3), and combinations thereof. In some cases, the anti-cancer agent is an IDO inhibitor. In some cases, the anti-cancer agent is a TDO inhibitor. In some cases, the anti-cancer agent is a CTLA-4 inhibitor.

TABLE 2
PD-1/PD-1L Inhibitors.
PD-1/PD-1L Inhibitor Compound Name

2-phenylbenzamidazole-5-sulfonic acid
2-propanediol dicarbanilate ester
abacavir
aceclofenac
acemetacin
acetohexamide
acetyltyrosine
acreozast
alacepril
alvimopan
aminoglutethimide
amisulpride
amprenavir
amsacrine
aprepitant
arformoterol
aspartame
aspoxicillin
astemizole
azlocillin
aztreonam
bacampecillin
benserazide
bentiromide
benzoxiquine
benzyl benzoate
benzyl nicotinate
benzylparaben
benzylpenicilloyl polylysine
beraprost
beta-d-glucopyranoside
betiatide
bisacodyl
boceprevir
butoctamide
butyl aminobenzoate
butylparaben
butyl-sympatol
calcitriol
calcium alginate
carfecillin
carindacillin
carumonam
cefanide
cefathiamidine
cefatrizine
cefazolin
cefetamet
cefixine
cefpiramide
ceftezole
ceftibuten
ceftiofur
ceftizoxime
ceftriaxone
cefuroxime
cefuroxime axetil
cefuzonam
chlamphenicol
chloprocaine
chlpropamide
cholestyramine
cidofovir
cisapride
citicoline
clindamycin
clinofibrate
cocarboxylase
cycotiamine
dasatinib
deferasirox
desoxycticosterone
dibucaine
diphenylcyclopropenone
dipivefrin
dobutamine
docarpamine
doxazosin
enalapril
ergometrine
esomeprazole
faropenem
fenoldopam
fenoterol
floctafenine
fluconazole
formoterol
gadodiamide
gastrodin
genistin
glafenine
gliclazide
glutathione
glybuzole
glyclopyramide
glycodiazine
gusperimus
ifenprodil
imatibin
imidurea
isoxsuprine
kalmegh
lansoprazole
lenampicillin
leucogen
levosulpride
lidofenin
lisdexamfetamine
lodoxamide
lofepramine
masoprocol
mefrusida
mesidazine
methotrexate
methyl salicyalate
methylergonovine
methysergide
metyrapone
micizine
midodrine
mizolastine
monobenzone
montirelin
mosapride
nafamostat
naratriptan
n-cyclohexyl-2-benzothiazylsulfenamide
nebivolol
nelfinavir
nibentan
nicardipine
nylidrin
osutidine
oxamniquine
panipenem
pentamidine
phenyl aminosalicylate
piperacillin
pivampicillin
podophyllum resin
procainamide
procaine
proglumide
propafenone
propyl paraben
protirelin
protokylol
quatenium 73
quinapril
racecadotril
rafoxanide
raloxifene
regadenoson
riboflavin
rilmazafone
ritodrine
sarpogrelate
seratrodast
sitagliptin
sitaxentan
sodium picosulphate
succinylsulfathiazole
sulalfadiazine
sulfabenzamide
sulfacetamide
sulfacytine
sulfadimethoxine
sulfadoxine
sulfamerazine
sulfameter
sulfamethizole
sulfamethoxazole
sulfamethoxypyridazine
sulfametomidine
sulfamonomethoxine
sulfanilamide
sulfaphenazole
sulfapyridine
sulfaquinoxaline
sulfatolamide
sulfisomidine
sulfisoxazole
sulfisoxazole acetyl
sulphaguanidine
sulpiride
sultopride
suxibuzone
talampicillin
taltirelin
technetium (tc-99m) mebrofenin
tetragastrin
thiothixene
tirofiban
tocofersolanum
tolbutamide
tranilast
ubenimex
vilazodone

In various aspects, compositions comprising one or more TDO/IDO inhibitors (e.g., 1, 2, 3, 4, 5, or more) further comprise and/or are administered with one or more IDO inhibitors (e.g., 1, 2, 3, 4, 5, or more). In some embodiments, the compositions comprising one or more TDO/IDO inhibitors and one or more IDO inhibitors are useful for the treatment of cancer. In some embodiments, the compositions comprising one or more TDO/IDO inhibitors administered with one or more IDO inhibitors are useful for the treatment of cancer. Non-limiting examples of IDO inhibitors useful in the compositions described herein include indoximod, F001287, NLG919, INCB024360, and salts and solvates thereof. Additional non-limiting examples of IDO inhibitors include the compounds listed in Table 1, and salts, solvates and combinations thereof. In some instances, the compositions comprising one or more TDO/IDO inhibitors and one or more IDO inhibitors further comprise one or more additional active agents. In some instances, the compositions comprising one or more TDO/IDO inhibitors administered with one or more IDO inhibitors are further administered with one or more additional active agents. Additional active agents include, without limitation, anti-cancer agents, PD-1 inhibitors (e.g., Table 2), TDO inhibitors (e.g., Table 1), CTLA-4 inhibitors (e.g., Table 3), and combinations thereof. In some cases, the anti-cancer agent is a PD-1 inhibitor. In some cases, the anti-cancer agent is a TDO inhibitor. In some cases, the anti-cancer agent is a CTLA-4 inhibitor.

In various aspects, compositions comprising one or more TDO/IDO inhibitors (e.g., 1, 2, 3, 4, 5, or more) further comprise and/or are administered with one or more CTLA-4 inhibitors (e.g., 1, 2, 3, 4, 5, or more). In some embodiments, the compositions comprising one or more TDO/IDO inhibitors and one or more CTLA-4 inhibitors are useful for the treatment of cancer. Non-limiting examples of CTLA-4 inhibitors include ipilimumab, tremelimumab, antibody clone 9H10, antibody clone BNI3, Del 60, and M9-14 del 55. Additional non-limiting examples of CTLA-4 inhibitors are provided in Table 3, and include salts, solvates, and combinations of the compounds of Table 3. In some embodiments, the compositions comprising one or more TDO/IDO inhibitors administered with one or more CTLA-4 inhibitors are useful for the treatment of cancer. In some instances, the compositions comprising one or more TDO/IDO inhibitors and one or more CTLA-4 inhibitors further comprise one or more additional active agents. In some instances, the compositions comprising one or more TDO/IDO inhibitors administered with one or more CTLA-4 inhibitors are further administered with one or more additional active agents. Additional active agents include, without limitation, anti-cancer agents, IDO inhibitors (e.g., Table 1), PD-1 inhibitors (e.g., Table 2), TDO inhibitors (e.g., Table 1), and combinations thereof. In some cases, the anti-cancer agent is an IDO inhibitor. In some cases, the anti-cancer agent is a PD-1 inhibitor. In some cases, the anti-cancer agent is a TDO inhibitor.

TABLE 3
CTLA-4 Inhibitors.
CTLA-4 Inhibitor Compound Name

1-(2-(2-methyl-4-(-o-tolyldiazenyl)phenyl)hydrazono)naphthalen-2(1h)-
one
1-(2-(naphth-2-yl)ethyl)-4-(3-trifluoromethylphenyl)-1,2,5,6-
tetrahydropyridine
y-23023, 2-(dimethylamino)-2-oxoethyl 2-(2-methyl-5H-chromeno[2,3-
b]pyridin-7-yl)propanoate
6-(2-(naphthalen-1-yl)hydrazono)-5-oxo-5,6-dihydronaphthalene-1-
sulfonic acid
abiraterone
abiraterone acetate
acemetacin
acetohexamide
alosetron
amyltricresol
apafant
azaperone
azelastine
balofloxacin
balsalazide
bendroflumethiazide
benzoyl peroxide
benzydamine
benzylhydrochlorothiazide
berberine
bicalutamide
brotizolam
bufogenin
carbamazepine
carfecillin
carindacillin
celecoxib
cepae ext
cetirizine
cga 184699 (lufenuron)
cilomilast
ciprofloxacin
clemizole
clopamide
cloxacillin
coumarin
crizotinib
cyclomethycaine
cyclopenthiazide
daidzein
dantrolene
dapiprazole
dasatinib
deferasirox
deracoxib
desoxycorticosterone
dicloxacillin
difloxacin
dolasetron
dolasetron mesylate
domperidone
donepezil
doxazosin
doxorubicin
droperidol
dyclonine
econazole
efloxate
eltrombopag
emedastine
enoxacin
ensulizole
epsiprantel
eslicarbazepine
estr-4-en-3-one, 17-(3-(2-furanyl)-1-oxopropoxy)-,(17beta)-)estr-4-en-3-
one
etizolam
etravirine
fabesetron
fenbufen
fenbutrazate
firocoxib
fleroxacin
flumazenil
flupirtine
flurbiprofen
flurbiprofen axetil
fluvastatin
folic acid
fp 83
gefitinib
gemifloxacin
gemifloxacin mesylate
gimatecan
grepafloxacin
he-11 (nibentan)
indapamide
indigo
ipriflavone
irinotecan
isoconazole
israpafant
lenalidomide
levofloxacin
linezolid
liranaftate
lomefloxacin
lornoxicam
marbofloxacin
mebhydrolin
meloxicam
mepitiostane
methyl polysilox
miconazole
mosapramine
moxifloxacin
mycophenolic acid
nadifloxacin
nandrolone cyclohexylpropionate
nandrolone phenylpropionate
naratriptan
nebivolol
nedocromil
nevirapine
nichicaine
niclosamide
nifenazone
nitazoxanide
norfloxacin
oestradiol benzoate
ofatumumab
ofloxacin
olanzapine
omoconazole
ondansetron
orbifloxacin
o-tolylazo-o-tolylazo-beta-naphthol
oxabolone
oxaprozin
oxcarbazepine
oxiconazole
oxitriptan
papaverine
perisoxal
pioglitazone
pipemidic acid
piperidolate
piperine
pirenoxine
piromidic acid
piroxicam
polythiazide
pranlukast
praziquantel
prazosin
procarbazine
protokylol
prulifloxacin
rafoxanide
ranolazine
regadenoson
repirinast
rivaroxaban
rubitecan
seratrodast
sertaconazole
silybin
simetride
sulbentinum
sulconazole
sulthiame
sunitinib
tamibarotene
terazosin
tiagabine
tiaprofenic acid
tilmacoxib
tinoridine
tolmetin
tolvaptan
tosufloxacin
troglitazone
vandetanib
vesnarinone
y-23023
y-3642
zaleplon
zaltoprofen
zolmitriptan

The compositions and TDO/IDO compounds described herein, in various aspects, are useful for the treatment of a disease or condition. Non-limiting examples of diseases or conditions include cancer and infectious diseases. In some embodiments, a composition comprising a TDO/IDO inhibitor inhibits tumor cell survival and/or promotes tumor cell death when administered to a patient having cancer. In some embodiments, a composition comprising a TDO/IDO inhibitor prevents and/or attenuates tumor cell growth when administered to a patient having cancer. In some embodiments, a composition comprising a TDO/IDO inhibitor prevents and/or attenuates tumor cell invasion when administered to a patient having cancer. In some embodiments, a composition comprising a TDO/IDO inhibitor prevents and/or attenuates tumor cell metastasis when administered to a patient having cancer.

In some embodiments, a composition provided herein comprises one or more TDO/IDO inhibitors and one or more additional active agents. In other or additional embodiments, a composition provided herein comprising one or more TDO/IDO inhibitors is co-administered with one or more additional active agents. In some embodiments, the composition is useful in a method of treating cancer, the method comprising administering the composition comprising the one or more TDO/IDO inhibitors and one or more additional active agents to a subject in need thereof. In some embodiments, the compositions is useful in a method of treating cancer, the method comprising co-administering the composition comprising the one or more TDO/IDO inhibitors with the one or more additional active agents. In some cases, the one or more additional active agents is a TDO/IDO inhibitor. In some cases, the one or more additional active agents is a CTLA-4 inhibitor, PD-1 inhibitor, IDO inhibitor, TDO inhibitor, or a combination thereof.

In various aspects of the disclosure, provided are compositions comprising one or more (e.g., 1, 2, 3, 4, 5 or more) TDO/IDO inhibitors comprising a compound of Table 1, or a salt, solvate or combination thereof. In some instances, the composition is administered with 1, 2, 3, or more additional TDO and/or IDO inhibitors. In some instances, the composition further comprises 1, 2, 3, or more additional TDO and/or IDO inhibitors. In some cases, the composition is administered with 1, 2, 3, or more PD-1 inhibitors. In some cases, the composition further comprises 1, 2, 3, or more PD-1 inhibitors. In some instances, the composition is administered with 1, 2, 3, or more CTLA-4 inhibitors. In some instances, the composition further comprises 1, 2, 3, or more CTLA-4 inhibitors. In some embodiments, the composition further comprises one or more PD-1 inhibitors, IDO inhibitors, TDO inhibitors, CTLA-4 inhibitors, or any combination thereof. In some embodiments, the composition is useful for the treatment of cancer. In some embodiments, the composition is useful for treating an infectious disease. In some embodiments, the composition further comprises and/or is administered with one or more anti-cancer agents, wherein an anti-cancer agent optionally comprises a PD-1 inhibitor, IDO inhibitor, CTLA-4 inhibitor, TDO inhibitor, or combinations thereof.

Provided herein, in various aspects, are compositions comprising one or more TDO/IDO inhibitors, wherein one or more of the TDO/IDO inhibitors are useful for the treatment and/or prevention of one or more diseases or conditions in addition to those described herein. For example, one or more TDO/IDO inhibitors are useful for the treatment of cancer and one or more additional diseases or conditions. In another example, one or more TDO/IDO inhibitors are useful for the treatment of an infectious disease and one or more additional diseases or conditions. In some embodiments, one or more TDO/IDO inhibitors are useful in compositions and methods that do not involve TDO/IDO inhibition.

In some instances, one or more TDO/IDO inhibitors of a composition herein are antimicrobial agents and are useful for the treatment of cancer. In some instances, one or more TDO/IDO inhibitors of a composition herein are antibacterial agents and are useful for the treatment of cancer and/or non-bacterial infectious disease. In some instances, one or more TDO/IDO inhibitors of a composition herein are antifungal agents and are useful for the treatment of cancer and/or non-fungal infectious disease. In some instances, one or more TDO/IDO inhibitors of a composition herein are antiparasitic agents and are useful for the treatment of cancer and/or non-parasitic infectious disease. In some instances, one or more TDO/IDO inhibitors of a composition herein are antiseptics and are useful for the treatment of cancer. In some instances, one or more TDO/IDO inhibitors of a composition herein are antiviral agents and are useful for the treatment of cancer and/or non-viral infectious disease. In some instances, one or more TDO/IDO inhibitors of a composition herein are sulfonamides, for example, diuretic and/or nonsteroidal anti-inflammatory sulfonamides, and are useful for the treatment of cancer and/or infectious disease. In some instances, one or more TDO/IDO inhibitors of a composition herein are nonsteroidal anti-inflammatory drugs (NSAIDs) and are useful for the treatment of cancer and/or infectious disease. In some instances, one or more TDO/IDO inhibitors of a composition herein are adrenergic agents and are useful for the treatment of cancer and/or infectious disease. In some instances, one or more TDO/IDO inhibitors of a composition herein are serotonergic agents and are useful for the treatment of cancer and/or infectious disease. In some instances, one or more TDO/IDO inhibitors of a composition herein are phosphodiesterase inhibitors and are useful for the treatment of cancer and/or infectious disease. In some instances, one or more TDO/IDO inhibitors of a composition herein are proton pump inhibitors and are useful for the treatment of cancer and/or infectious disease. In some instances, one or more TDO/IDO inhibitors of a composition herein are acetocholinesterase inhibitors, adrogenic agents, coumarins, anti-inflammatory drugs, antipsychotic agents, dopamenergic agents, GABA modulators, laxatives, muscle relaxants, sigmaergic agents, vasodilators, vitamins, antioxidants, or combinations thereof, and are useful for the treatment of cancer and/or infectious disease. In some instances, one or more TDO/IDO inhibitors of a composition herein also function as inhibitors of a PD-1 pathway. In some instances, one or more TDO/IDO inhibitors of a composition herein also function as CTLA-4 inhibitors. Non-limiting examples of TDO/IDO inhibitors of a composition provided herein include the compounds of Table 1 and the salts and solvates of the compounds of Table 1.

Provided herein, in various aspects, are compositions comprising an antimicrobial agent. In some embodiments, the compositions further comprise and/or are administered with one or more of the following additional active agents: an additional antimicrobial agent, adrenergic agent, serotonergic agent, NSAID, sulfonamide, phosphodiesterase inhibitor, proton pump inhibitor, acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, any active agent comprising a compound from Table 1, a salt or solvate of any active agent comprising a compound from Table 1, or combination thereof. In exemplary embodiments, the antimicrobial agent is a TDO/IDO inhibitor. In some cases, one of the one or more additional active agents is a TDO/IDO inhibitor. In some embodiments, the composition further comprises and/or is administered with 1, 2, 3, 4, 5, or more CTLA-4 inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more TDO inhibitors. In some embodiments, the composition further comprises and/or is administered with one or more CTLA-4 inhibitors, PD-1 inhibitors, IDO inhibitors, TDO inhibitors, or a combination thereof. In some cases, the composition is useful for the treatment of cancer. In some cases, the composition further comprises and/or is administered with an anti-cancer agent. In some cases, the composition is useful for the treatment of an infectious disease. In some embodiments, the antimicrobial agent comprises an antibacterial agent. In some embodiments, the antibacterial agent comprises cloxiquine, betamipronum, chloroxine, nalidixic acid, pazufloxacin, propicillin, besifloxacin, oxacillin sodium, moxifloxacin, cloxacillin, dicloxacillin, or a salt, solvate or combination thereof. In some embodiments, the antimicrobial agent comprises an antifungal agent. In some embodiments, the antifungal agent comprises tioconazole, liarozole, sertaconazole, econazole, sulconazole, miconazole, isoconazole, itraconazole, fenticonazole, cloconazole, acrisorcinum, climbazole, croconazole, diphenylimidazole, exalamide, lanoconazole, oxiconazole, bifonazole, luliconazole, thiabendazole, butoconazole, clotrimazole, ketoconazole, luliconazole, albaconazole, efinaconazole, epoxiconazole, fluconazole, isavuconazole, posaconazole, propiconazole, ravuconazole, terconazole, voriconazole, abafungin, omoconazole, phenoxyacetic acid, naftifine, tolindate, tolnaftate, butenafine, o-(5,6,7,8-tetrahydro-2-naphthyl)-6-methoxy-n-methylthio-2-pyridinecarbamate, or a salt, solvate or combination thereof. In some embodiments, the antimicrobial agent comprises an antiparasitic agent. In some embodiments, the antiparasitic agent comprises praziquantel, chloroquine, epsiprantel, niclosamide, hydroxychloroquin, or a salt, solvate or combination thereof. In some embodiments, the antimicrobial agent comprises an antiseptic. In some embodiments, the antiseptic comprises iodochlorohydroxyquinoline, euresol, acrisorcinum, benzyl peroxide, benzoyl peroxide, benzoxiquine, ethacridine lactate, or a salt, solvate or combination thereof. In some embodiments, the antimicrobial agent comprises an antiviral agent. In some embodiments, the antiviral agent comprises imiquimod, efavirenz, or a salt, solvate or combination thereof. In some embodiments, the antimicrobial agent comprises chlorquinaldol, or a salt or solvate thereof.

Provided herein, in various aspects, are compositions comprising an adrenergic agent. In some embodiments, the compositions further comprise and/or are administered with one or more of the following additional active agents: antimicrobial agent, additional adrenergic agent, serotonergic agent, NSAID, sulfonamide, phosphodiesterase inhibitor, proton pump inhibitor, acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, any active agent comprising a compound from Table 1, a salt or solvate of any active agent comprising a compound from Table 1, or combination thereof. In exemplary embodiments, the adrenergic agent is a TDO/IDO inhibitor. In some cases, one of the one or more additional active agents is a TDO/IDO inhibitor. In some embodiments, the composition further comprises and/or is administered with 1, 2, 3, 4, 5, or more CTLA-4 inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more TDO inhibitors. In some embodiments, the composition further comprises and/or is administered with one or more CTLA-4 inhibitors, PD-1 inhibitors, IDO inhibitors, TDO inhibitors, or a combination thereof. In some cases, the composition is useful for the treatment of cancer. In some cases, the composition further comprises and/or is administered with an anti-cancer agent. In some cases, the composition is useful for the treatment of an infectious disease. In some embodiments, the adrenergic agent comprises octopamine, ephedrine, epinephrine, levonordefrin, norfenefrine, methoxamine, mirtazapine, norpseudoephedrine, pseudophedrine, norepinephrine, adrenochrome, amezinium, clobenzorex, mirtazapine, or a salt, solvate or combination thereof.

Provided herein, in various aspects, are compositions comprising a serotonergic agent. In some embodiments, the compositions further comprise and/or are administered with one or more of the following additional active agents: antimicrobial agent, adrenergic agent, additional serotonergic agent, NSAID, sulfonamide, phosphodiesterase inhibitor, proton pump inhibitor, acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, any active agent comprising a compound from Table 1, a salt or solvate of any active agent comprising a compound from Table 1, or combination thereof. In exemplary embodiments, the serotonergic agent is a TDO/IDO inhibitor. In some cases, one of the one or more additional active agents is a TDO/IDO inhibitor. In some embodiments, the composition further comprises and/or is administered with 1, 2, 3, 4, 5, or more CTLA-4 inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more TDO inhibitors. In some embodiments, the composition further comprises and/or is administered with one or more CTLA-4 inhibitors, PD-1 inhibitors, IDO inhibitors, TDO inhibitors, or a combination thereof. In some cases, the composition is useful for the treatment of cancer. In some cases, the composition further comprises and/or is administered with an anti-cancer agent. In some cases, the composition is useful for the treatment of an infectious disease. In some embodiments, the serotonergic agent comprises 1-tryptophan, frovatriptan succinate, ramosetron, mazindol, ondansetron, itasetron, indisetron, naratriptan, or a salt, solvate or combination thereof.

Provided herein, in various aspects, are compositions comprising a sulfonamide. In some embodiments, the compositions further comprise and/or are administered with one or more of the following additional active agents: antimicrobial agent, adrenergic agent, serotonergic agent, NSAID, additional sulfonamide, phosphodiesterase inhibitor, proton pump inhibitor, acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, any active agent comprising a compound from Table 1, a salt or solvate of any active agent comprising a compound from Table 1, or combination thereof. In exemplary embodiments, the sulfonamide is a TDO/IDO inhibitor. In some cases, one of the one or more additional active agents is a TDO/IDO inhibitor. In some embodiments, the composition further comprises and/or is administered with 1, 2, 3, 4, 5, or more CTLA-4 inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more TDO inhibitors. In some embodiments, the composition further comprises and/or is administered with one or more CTLA-4 inhibitors, PD-1 inhibitors, IDO inhibitors, TDO inhibitors, or a combination thereof. In some cases, the composition is useful for the treatment of cancer. In some cases, the composition further comprises and/or is administered with an anti-cancer agent. In some cases, the composition is useful for the treatment of an infectious disease. In some embodiments, the sulfonamide is a diuretic. In some embodiments, the sulfonamide comprises furosemide, chlorthalidone, indapamide, cyclopenthiazide, alilusem, benzthiazide, polythiazide, or a salt, solvate or combination thereof.

Provided herein, in various aspects, are compositions comprising a NSAID. In some embodiments, the compositions further comprise and/or are administered with one or more of the following additional active agents: antimicrobial agent, adrenergic agent, serotonergic agent, additional NSAID, sulfonamide, phosphodiesterase inhibitor, proton pump inhibitor, acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, any active agent comprising a compound from Table 1, a salt or solvate of any active agent comprising a compound from Table 1, or combination thereof. In exemplary embodiments, the NSAID is a TDO/IDO inhibitor. In some cases, one of the one or more additional active agents is a TDO/IDO inhibitor. In some embodiments, the composition further comprises and/or is administered with 1, 2, 3, 4, 5, or more CTLA-4 inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more TDO inhibitors. In some embodiments, the composition further comprises and/or is administered with one or more CTLA-4 inhibitors, PD-1 inhibitors, IDO inhibitors, TDO inhibitors, or a combination thereof. In some cases, the composition is useful for the treatment of cancer. In some cases, the composition further comprises and/or is administered with an anti-cancer agent. In some cases, the composition is useful for the treatment of an infectious disease. In some embodiments, the NSAID comprises glucosamine, nabumetone, fenoprofen, loxoprofen, diflunisal, ketoprofen, dexketoprofen, fenbufen, nepafenac, tolmetin, pelubiprofen, tiaprofenic acid, suprofen, metiazinic acid, carprofen, clidanac, etodolac, zaltoprofen, nifenazone, rofecoxib, protizinic acid, methyl salicylate, piroxicam, meloxicam, diacerein, lornoxicam, glafenine, or a salt, solvate, or combination thereof.

Provided herein, in various aspects, are compositions comprising a phosphodiesterase inhibitor. In some embodiments, the compositions further comprise and/or are administered with one or more of the following additional active agents: antimicrobial agent, adrenergic agent, serotonergic agent, NSAID, sulfonamide, additional phosphodiesterase inhibitor, proton pump inhibitor, acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, any active agent comprising a compound from Table 1, a salt or solvate of any active agent comprising a compound from Table 1, or combination thereof. In exemplary embodiments, the phosphodiesterase inhibitor is a TDO/IDO inhibitor. In some cases, one of the one or more additional active agents is a TDO/IDO inhibitor. In some embodiments, the composition further comprises and/or is administered with 1, 2, 3, 4, 5, or more CTLA-4 inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more TDO inhibitors. In some embodiments, the composition further comprises and/or is administered with one or more CTLA-4 inhibitors, PD-1 inhibitors, IDO inhibitors, TDO inhibitors, or a combination thereof. In some cases, the composition is useful for the treatment of cancer. In some cases, the composition further comprises and/or is administered with an anti-cancer agent. In some cases, the composition is useful for the treatment of an infectious disease. In some embodiments, the phosphodiesterase inhibitor comprises theophylline, ibudilast, anagrelide, doxofylline, rolipram, cilomilast, vinpocetine, or a salt, solvate or combination thereof.

Provided herein, in various aspects, are compositions comprising a proton pump inhibitor. In some embodiments, the compositions further comprise and/or are administered with one or more of the following additional active agents: antimicrobial agent, adrenergic agent, serotonergic agent, NSAID, sulfonamide, phosphodiesterase inhibitor, additional proton pump inhibitor, acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, any active agent comprising a compound from Table 1, a salt or solvate of any active agent comprising a compound from Table 1, or combination thereof. In exemplary embodiments, the proton pump inhibitor is a TDO/IDO inhibitor. In some cases, one of the one or more additional active agents is a TDO/IDO inhibitor. In some embodiments, the composition further comprises and/or is administered with 1, 2, 3, 4, 5, or more CTLA-4 inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more TDO inhibitors. In some embodiments, the composition further comprises and/or is administered with one or more CTLA-4 inhibitors, PD-1 inhibitors, IDO inhibitors, TDO inhibitors, or a combination thereof. In some cases, the composition is useful for the treatment of cancer. In some cases, the composition further comprises and/or is administered with an anti-cancer agent. In some cases, the composition is useful for the treatment of an infectious disease. In some embodiments, the proton pump inhibitor comprises esomeprazole, omeprazole, tenatoprazole, rabeprazole, lansoprazole, pantoprazole, or a salt, solvate or combination thereof.

Provided herein, in various aspects, are compositions comprising an acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, or a combination thereof. In some embodiments, the compositions further comprise and/or are administered with one or more of the following additional active agents: antimicrobial agent, adrenergic agent, serotonergic agent, NSAID, sulfonamide, phosphodiesterase inhibitor, proton pump inhibitor, any active agent comprising a compound from Table 1, a salt or solvate of any active agent comprising a compound from Table 1, or combination thereof. In exemplary embodiments, the acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, and/or antioxidant is a TDO/IDO inhibitor. In some cases, one of the one or more additional active agents is a TDO/IDO inhibitor. In some embodiments, the composition further comprises and/or is administered with 1, 2, 3, 4, 5, or more CTLA-4 inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more TDO inhibitors. In some embodiments, the composition further comprises and/or is administered with one or more CTLA-4 inhibitors, PD-1 inhibitors, IDO inhibitors, TDO inhibitors, or a combination thereof. In some cases, the composition is useful for the treatment of cancer. In some cases, the composition further comprises and/or is administered with an anti-cancer agent. In some cases, the composition is useful for the treatment of an infectious disease. In some embodiments, the acetocholinesterase inhibitor comprises edrophonium, tacrine, or a salt, solvate or combination thereof. In some embodiments, the adrogenic agent comprises trenbolone acetate, abiraterone acetate, or a salt, solvate or combination thereof. In some embodiments, the coumarin comprises coumarin, cantabiline, methoxsalen, phenprocoumon, or a salt, solvate or combination thereof. In some embodiments, the anti-inflammatory drug comprises mesalazine, felbinac ethyl ester, perisoxalum, or a salt, solvate or combination thereof. In some embodiments, the antipsychotic agent comprises blonanserin, acetophenazine, carfenazine, pimozide, or a salt, solvate or combination thereof. In some embodiments, the dopamenergic agent comprises zelandopam, droperidol, benperidol, domperidone, or a salt, solvate or combination thereof. In some embodiments, the GABA modulator comprises zaleplon, cyclothiazide, or a salt, solvate or combination thereof. In some embodiments, the laxative comprises 1,8-dihydroxyanthraquinone, emodin, or a salt, solvate or combination thereof. In some embodiments, the muscle relaxant comprises chlorzoxazone, methocarbamol, chlorphenesin carbamate, tolperisone, or a salt, solvate or combination thereof. In some embodiments, the sigmaergic agent comprises berberine, noscapine, or a salt, solvate or combination thereof. In some embodiments, the vasodilator comprises trapidil, cyclandelate, diazoxide, or a salt, solvate or combination thereof. In some embodiments, the vitamin comprises pyridoxine, menadione, caldibasic phosphate, benzyl nicotinate, cycotiamine, d-panthenol, or a salt, solvate or combination thereof. In some embodiments, the antioxidant comprises edaravone, alphalipoic acid, lipoic acid, santoquin, melatonin, or a salt, solvate or combination thereof.

Provided herein, in various aspects, are compositions comprising a compound, or a salt or solvate thereof, of Table 1. In some embodiments, the compositions further comprise and/or are administered with one or more of the following additional active agents: antimicrobial agent, adrenergic agent, serotonergic agent, NSAID, sulfonamide, phosphodiesterase inhibitor, proton pump inhibitor, acetocholinesterase inhibitor, adrogenic agent, coumarin, anti-inflammatory drug, antipsychotic agent, dopamenergic agent, GABA modulator, laxative, muscle relaxant, sigmaergic agent, vasodilator, vitamin, antioxidant, any additional active agent comprising a compound from Table 1, or a salt, solvate or combination thereof. In exemplary embodiments, the compound of Table 1 is a TDO/IDO inhibitor. In some cases, one of the one or more additional active agents is a TDO/IDO inhibitor. In some embodiments, the composition further comprises and/or is administered with 1, 2, 3, 4, 5, or more TDO inhibitors. In some embodiments, the composition further comprises and/or is administered with 1, 2, 3, 4, 5, or more PD-1 inhibitors. In some embodiments, the composition further comprises and/or is administered with 1, 2, 3, 4, 5, or more IDO inhibitors. In some embodiments, the composition further comprises and/or is administered with 1, 2, 3, 4, 5, or more CTLA-4 inhibitors. In some embodiments, the composition further comprises and/or is administered with one or more CTLA-4 inhibitors, PD-1 inhibitors, IDO inhibitors, TDO inhibitors or a combination thereof. In some cases, the composition is useful for the treatment of cancer. In some cases, the composition further comprises and/or is administered with an anti-cancer agent. In some cases, the composition is useful for the treatment of an infectious disease.

### Methods of Treatment

Provided herein, in various embodiments, are methods of treating a disease or condition comprising the administration of a composition comprising one or more TDO/IDO inhibitors. Non-limiting examples of TDO/IDO inhibitors for treating a disease or condition are provided in Table 1 and include salts and solvates of the compounds in Table 1. In some instances, a composition comprising two or more TDO/IDO inhibitors is useful for treating a disease or condition. In some instances, a composition comprising 3, 4, 5 or more TDO/IDO inhibitors is useful for treating a disease or condition. In some cases, the disease is cancer. In some embodiments, the TDO/IDO inhibitors provided herein are useful in immunotherapy, stem cell therapy, and/or CAR T-cell therapy. As used herein, use of a TDO/IDO inhibitor for the treatment of cancer as described herein extends to immunotherapies, stem cell therapies, and CAR T-cell therapies, without limitation. Accordingly, any TDO/IDO inhibitor described herein is useful for immunotherapies, stem cell therapies, and/or CAR T-cell therapies. In some cases, the disease is an infectious disease. In some instances, the methods further comprise the administration of one or more active agents, where the one or more active agents are administered with the one or more TDO/IDO inhibitors together or separately from the one or more TDO/IDO inhibitors. In some instances, the composition comprising the one or more TDO/IDO inhibitors further comprises one or more additional active agents. In some embodiments, an additional active agent is a PD-1 inhibitor. In some embodiments, an additional active agent is an IDO inhibitor. In some embodiments, an additional active agent is a TDO inhibitor. In some embodiments, an additional active agent is a CTLA-4 inhibitor. In some cases, the methods further comprise the administration of one or more anti-cancer therapies, wherein the one or more anti-cancer therapies is administered with or separately from the one or more TDO/IDO inhibitors. In some cases, the composition comprising the one or more TDO/IDO inhibitors further comprises one or more anti-cancer agents. In some cases, an anti-cancer agent is a PD-1 inhibitor, IDO inhibitor, CTLA-4 inhibitor, TDO inhibitor, or any combination thereof. In some embodiments, a TDO/IDO inhibitor useful in the compositions and methods described herein comprises a compound of Table 1, or a salt or solvate thereof. Additional non-limiting examples of TDO/IDO inhibitors useful in the compositions and methods described herein include indoximod, F001287, NLG919 (D-1MT), INCB024360, or a salt, solvate or combination thereof. Non-limiting examples of PD-1 inhibitors useful in the compositions and methods described herein include BMS-936559, MEDI4736, MPDL3280A, MSB0010718C, AMP-224, nivolumab, pembrolizumab, pidilizumab, BMS-986016, MDX1105-01, avelumab, compounds of Table 2, and salts and solvates thereof. Non-limiting examples of IDO inhibitors useful in the compositions and methods described herein include indoximod, F001287, NLG919, INCB024360, and salts and solvates thereof. Non-limiting examples of CTLA-4 inhibitors useful in the compositions and methods described herein include ipilimumab, tremelimumab, antibody clone 9H10, antibody clone BNI3, Del 60, M9-14 del 55, compounds of Table 3, and salts and solvates thereof.

A composition described herein is useful for the treatment of diseases and conditions in subjects and patients that include humans and non-human mammals (e.g., mice, rats, pigs, cats, dogs, horses). Additional subjects include, without limitation, livestock such as cattle, sheep, goats, swine; poultry such as chickens, ducks, geese, turkeys; and domesticated animals such as dogs and cats. In addition, subjects include, without limitation, subjects suitable for diagnostic or research applications. Additional subjects include, without limitation, rodents such as mice, rats and hamsters; rabbits; primates and swine such as inbred pigs. The terms do not denote a particular age. Thus, both adult and children are intended to be covered.

In many instances, a treatment is an act upon a disease or condition with a composition to reduce or ameliorate the pharmacologic and/or physiologic effects of the disease or condition and/or its symptoms. In some examples, the disease is cancer. In some embodiments, treatment includes reducing the risk of occurrence of a disease in a subject determined to be predisposed to the disease but not yet diagnosed as infected with the disease. In this instance, a treatment may be considered a way of inhibiting said disease. In some embodiments, treatment includes impeding the development of a disease. In other or additional embodiments, treatment includes relieving a disease by causing regression of the disease and/or relief from one or more disease symptoms. Treatment, in many implementations, includes the delivery of an agent to provide a pharmacologic effect, even in the absence of a disease or condition. In various embodiments, treatment comprises administration of a composition comprising at least one active agent. In exemplary embodiments, a TDO/IDO inhibitor is an active agent of a composition described herein. In some embodiments, a PD-1 inhibitor is an active agent of a composition described herein. In some embodiments, an IDO inhibitor is an active agent of a composition described herein. In some embodiments, a CTLA-4 inhibitor is an active agent of a composition described herein. In some embodiments, a TDO inhibitor is an active agent of a composition described herein. In some embodiments, an anti-cancer agent is an active agent of a composition described herein.

In one aspect, provided herein is a method of treating cancer comprising administering to a patient in need thereof an effective amount of a compound of Formula (I), or a pharmaceutically acceptable salt or solvate thereof:

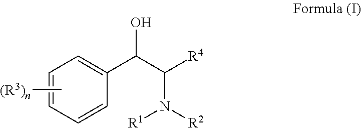

wherein
R¹ and R² are each independently selected from hydrogen or C₁-C₆ alkyl;
each R³ are independently selected from hydrogen, halogen, —OH, C₁-C₆ alkyl, or C₁-C₆ alkoxy;
R⁴ is hydrogen, halogen, or C₁-C₆ alkyl; and
n is 1, 2, 3, or 4.

In some embodiments, R¹ and R² are each hydrogen. In some embodiments, R¹ is hydrogen and R² is methyl. In some embodiments, R⁴ is hydrogen. In some embodiments, R⁴ is methyl. In some embodiments, n is 1 and R³ is hydrogen. In some embodiments, n is 1 and R³ is —OH. In some embodiments, n is 2 and R³ are each —OH. In some embodiments, n is 2 and R³ are each —OMe. In some embodiments, the compound of Formula (I) or a pharmaceutically acceptable salt or solvate comprises:

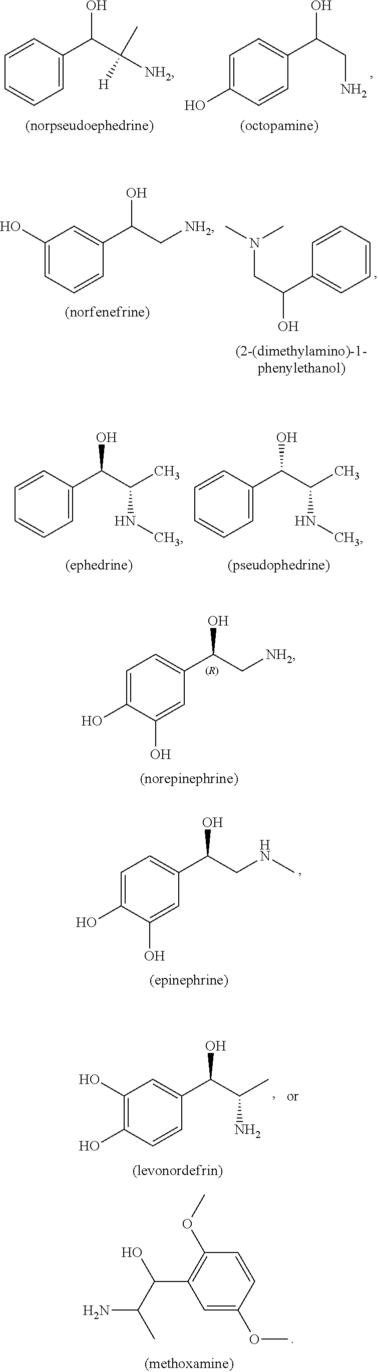

In some embodiments, a method of treating a disease comprises administering to a patient in need thereof an effective amount of a compound of Formula (I), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (I) is a TDO/IDO inhibitor, i.e. the compound of Formula (I) is a TDO inhibitor, IDO inhibitor, or an inhibitor of both TDO and IDO. In some embodiments, the compound of Formula (I) is not a TDO inhibitor. In some embodiments, a compound of Formula (I) is not an IDO inhibitor. In some embodiments, a compound of Formula (I) is not an inhibitor of both TDO and IDO. In some embodiments, the disease is cancer.

In another aspect, provided herein is a method of treating cancer comprising administering to a patient in need thereof an effective amount of a compound of Formula (II), or a pharmaceutically acceptable salt or solvate thereof:

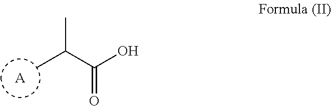

wherein
Ring A is optionally substituted aryl or optionally substituted heteroaryl.

In some embodiments, Ring A is optionally substituted heteroaryl. In some embodiments, Ring A is optionally substituted aryl. In some embodiments, the compound of Formula (II) is of Formula (IIa):

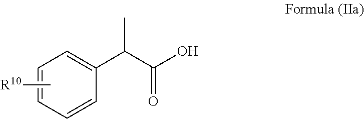

wherein
R¹⁰ is —X—R¹¹;
X is —O—, —CH₂—, —(C═O)—, or —CH═; and
R¹¹ is optionally substituted aryl, optionally substituted cycloalkyl, optionally substituted heteroaryl, or optionally substituted heterocycloalkyl.

In some embodiments, R¹¹ is phenyl. In some embodiments, X is —O— or —(C═O)—. In some embodiments, the compound of Formula (II) or a pharmaceutically acceptable salt or solvate comprises:

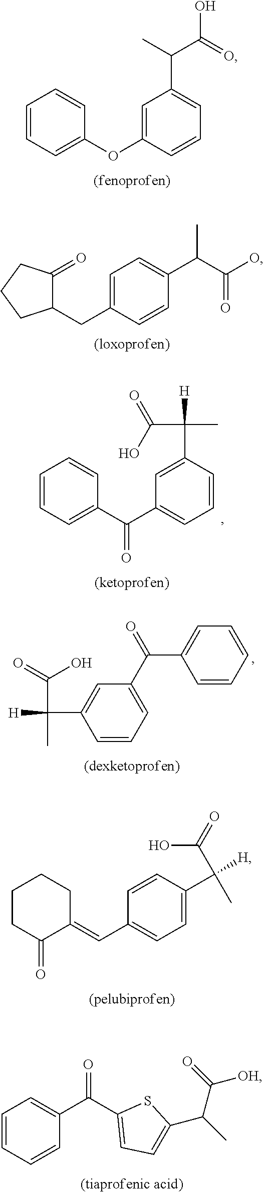

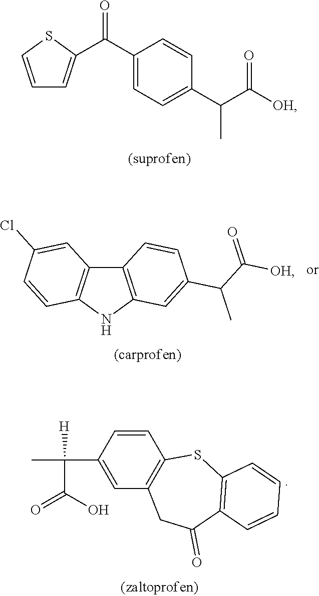 [SMILES: CC(C(=O)O)c1ccc(C(=O)c2cccs2)cc1 ; CC(C(=O)O)c1ccc2c(c1)[nH]c1ccc(Cl)cc12 ; "or" ; C[C@H](C(=O)O)c1ccc2c(c1)CC(=O)c1ccccc1S2]

In some embodiments, a method of treating a disease comprises administering to a patient in need thereof an effective amount of a compound of Formula (II), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (I) is a TDO/IDO inhibitor, i.e. the compound of Formula (II) is a TDO inhibitor, IDO inhibitor, or an inhibitor of both TDO and IDO. In some embodiments, the compound of Formula (II) is not a TDO inhibitor. In some embodiments, a compound of Formula (II) is not an IDO inhibitor. In some embodiments, a compound of Formula (II) is not an inhibitor of both TDO and IDO. In some embodiments, the disease is cancer.

In another aspect, provided herein is a method of treating cancer comprising administering to a patient in need thereof an effective amount of a compound of Formula (III), or a pharmaceutically acceptable salt or solvate thereof:

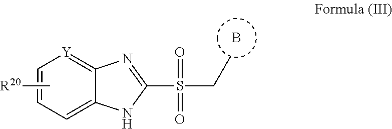

wherein
Ring B is an optionally substituted heteroaryl;
Y is —CH— or —N—; and R²⁰ is hydrogen, halogen, C₁-C₆ alkyl, C₁-C₆ alkoxy, or C₁-C₆ haloalkoxy.

In some embodiments, Ring B is optionally substituted pyridyl. In some embodiments, the compound of Formula (III) is of Formula (IIIa):

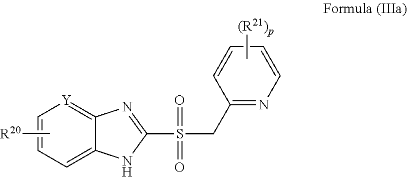

wherein
each R²¹ is independently hydrogen, C₁-C₆ alkyl, C₁-C₆ haloalkyl, C₁-C₆ alkoxy, alkoxy C₁-C₆alkoxy, or C₁-C₆ haloalkoxy; and
p is 1, 2, 3, or 4.

In some embodiments, p is 2. In some embodiments, p is 3. In some embodiments, each R²¹ is independently hydrogen, methyl, methoxy, 2,2,2-trifluoroethoxy, or 2-methoxypropoxy. In some embodiments, Y is —CH—. In some embodiments, R²⁰ is hydrogen, methoxy, or difluoromethoxy. In some embodiments, the compound of Formula (III) or a pharmaceutically acceptable salt or solvate comprises:

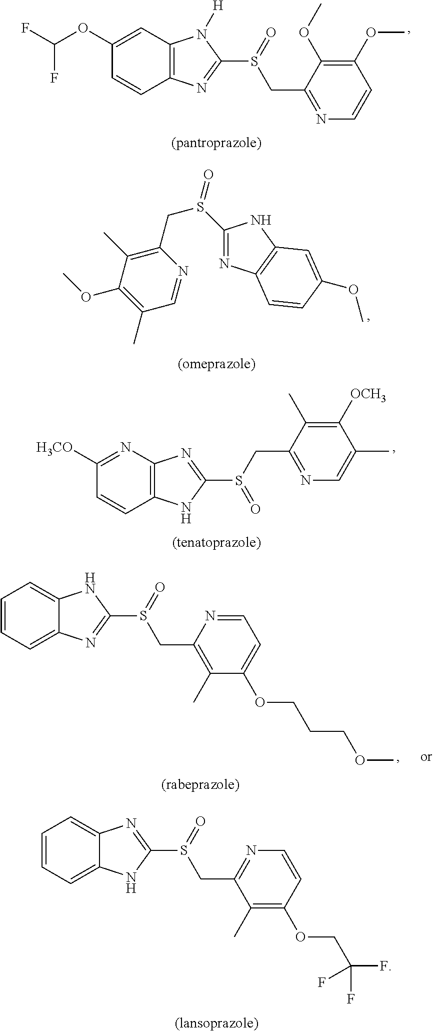

In some embodiments, a method of treating a disease comprises administering to a patient in need thereof an effective amount of a compound of Formula (III), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (III) is a TDO/IDO inhibitor, i.e. the compound of Formula (III) is a TDO inhibitor, IDO inhibitor, or an inhibitor of both TDO and IDO. In some embodiments, the compound of Formula (III) is not a TDO inhibitor. In some embodiments, a compound of Formula (III) is not an IDO inhibitor. In some embodiments, a compound of Formula (III) is not an inhibitor of both TDO and IDO. In some embodiments, the disease is cancer.

In another aspect, provided herein is a method of treating cancer comprising administering to a patient in need thereof an effective amount of a compound of Formula (IV), or a pharmaceutically acceptable salt or solvate thereof:

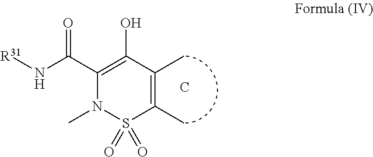

wherein
R³¹ is an optionally substituted aryl or an optionally substituted heteroaryl;
Ring C is an optionally substituted aryl or an optionally substituted heteroaryl.

In some embodiments, Ring C is optionally substituted phenyl or optionally substituted thiophenyl. In some embodiments, Ring C in phenyl. In some embodiments, Ring C in optionally substituted thiophenyl. In some embodiments, R³¹ is optionally substituted heteroaryl. In some embodiments, R³¹ is pyridyl or methyl substituted thiazolyl. In some embodiments, the compound of Formula (IV) or a pharmaceutically acceptable salt or solvate comprises:

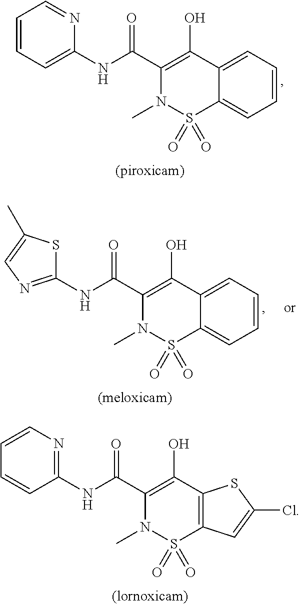 [SMILES: CN1C(C(=O)Nc2ccccn2)=C(O)c2ccccc2S1(=O)=O ; Cc1cnc(NC(=O)C2=C(O)c3ccccc3S(=O)(=O)N2C)s1 ; CN1C(C(=O)Nc2ccccn2)=C(O)c2sc(Cl)cc2S1(=O)=O]

In some embodiments, a method of treating a disease comprises administering to a patient in need thereof an effective amount of a compound of Formula (IV), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (IV) is a TDO/IDO inhibitor, i.e. the compound of Formula (IV) is a TDO inhibitor, IDO inhibitor, or an inhibitor of both TDO and IDO. In some embodiments, the compound of Formula (IV) is not a TDO inhibitor. In some embodiments, a compound of Formula (IV) is not an IDO inhibitor. In some embodiments, a compound of Formula (IV) is not an inhibitor of both TDO and IDO. In some embodiments, the disease is cancer.

In another aspect, provided herein is a method of treating cancer comprising administering to a patient in need thereof an effective amount of a compound of Formula (V), or a pharmaceutically acceptable salt or solvate thereof:

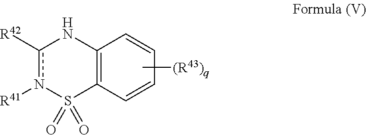

wherein
[symbol] is a single bond or a double bond;
R⁴¹ is hydrogen or C₁-C₆ alkyl when [symbol] is a single bond; or
R⁴¹ is absent when [symbol] is a double bond;
R⁴² is C₁-C₆ alkyl, optionally substituted aryl, optionally substituted aralkyl, optionally substituted heteroaryl, optionally substituted heteroaralkyl, optionally substituted cycloalkyl, optionally substituted cycloalkylalkyl, or —(CH₂)—S—R⁴⁴;
each R⁴³ is independently hydrogen, halogen, C₁-C₆ alkyl, C₁-C₆ alkoxy, or —S(═O)₂NH₂;
R⁴⁴ is C₁-C₆ alkyl, C₁-C₆ haloalkyl, or optionally substituted aralkyl; and
q is 1, 2, 3, or 4.

In some embodiments, the compound of Formula (V) is of Formula (Va):

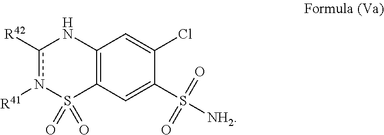

In some embodiments, [symbol] is a single bond and R⁴¹ is hydrogen. In some embodiments, [symbol] is a single bond and R⁴¹ is methyl. In some embodiments, [symbol] is a double bond and R⁴¹ is absent. In some embodiments, R⁴² is optionally substituted aralkyl, optionally substituted cycloalkyl, or optionally substituted cycloalkylalkyl. In some embodiments, R⁴² is benzyl, cyclopentylmethyl, or bicyclo[2.2.1]hept-2-ene. In some embodiments, R⁴² is —(CH₂)—S—R⁴⁴. In some embodiments, R⁴⁴ is C₁-C₆ haloalkyl, or optionally substituted aralkyl. In some embodiments, R⁴⁴ is benzyl or 2,2,2-trifluoroethyl. In some embodiments, the compound of Formula (V) or a pharmaceutically acceptable salt or solvate comprises:

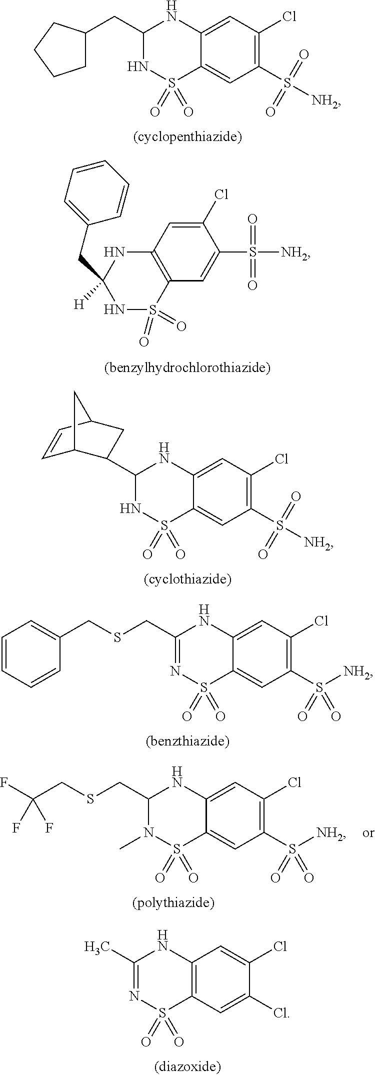 [SMILES: NS(=O)(=O)c1cc2c(cc1Cl)NC(CC1CCCC1)NS2(=O)=O ; NS(=O)(=O)c1cc2c(cc1Cl)N[C@H](Cc1ccccc1)NS2(=O)=O ; NS(=O)(=O)c1cc2c(cc1Cl)NC(C1CC3C=CC1C3)NS2(=O)=O ; NS(=O)(=O)c1cc2c(cc1Cl)NC(CSCc1ccccc1)=NS2(=O)=O ; CN1C(CSCC(F)(F)F)Nc2cc(Cl)c(S(N)(=O)=O)cc2S1(=O)=O ; CC1=NS(=O)(=O)c2cc(Cl)c(Cl)cc2N1]

In some embodiments, a method of treating a disease comprises administering to a patient in need thereof an effective amount of a compound of Formula (V), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (V) is a TDO/IDO inhibitor, i.e. the compound of Formula (V) is a TDO inhibitor, IDO inhibitor, or an inhibitor of both TDO and IDO. In some embodiments, the compound of Formula (V) is not a TDO inhibitor. In some embodiments, a compound of Formula (V) is not an IDO inhibitor. In some embodiments, a compound of Formula (V) is not an inhibitor of both TDO and IDO. In some embodiments, the disease is cancer.

In another aspect, provided herein is a method of treating cancer comprising administering to a patient in need thereof an effective amount of a compound of Formula (VI), or a pharmaceutically acceptable salt or solvate thereof:

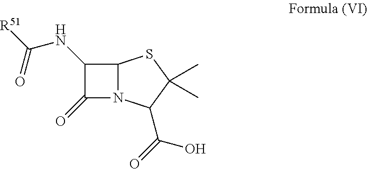

wherein
R⁵¹ is optionally substituted aryl, optionally substituted heteroaryl, or —CHR⁵²R⁵³;
R⁵² is C₁-C₆ alkyl; and
R⁵³ is optionally substituted aryl, optionally substituted heteroaryl, or optionally substituted aryloxy.

In some embodiments, R⁵¹ is optionally substituted isoxazolyl. In some embodiments, the compound of Formula (VI) is of Formula (VIa):

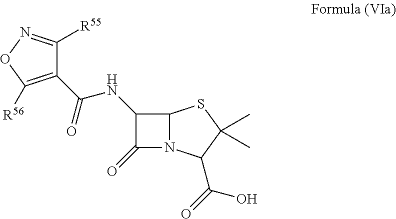

wherein
R⁵⁴ is C₁-C₆ alkyl; and
R⁵⁵ is optionally substituted aryl.

In some embodiments, R⁵⁵ is optionally substituted phenyl. In some embodiments, R⁵⁴ is methyl. In some embodiments, R⁵¹ is —CHR⁵²R⁵³. In some embodiments, R⁵² is phenyl and R⁵³ is —C(═O)OR⁵⁴. In some embodiments, the compound of Formula (VI) or a pharmaceutically acceptable salt or solvate comprises:

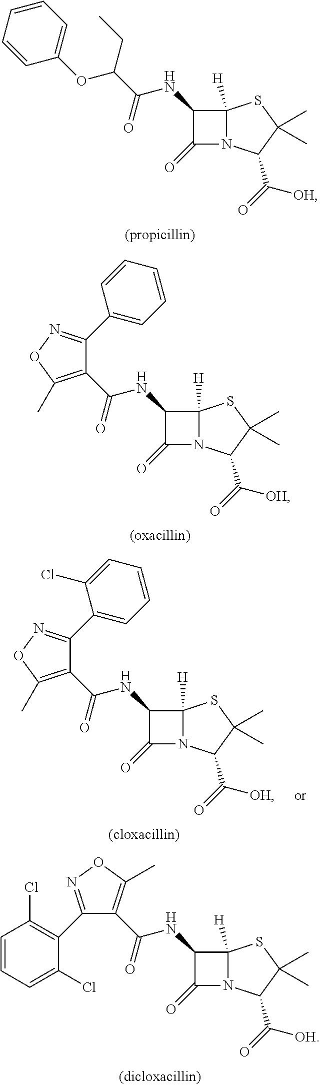 [SMILES: CCC(Oc1ccccc1)C(=O)N[C@@H]1C(=O)N2[C@@H]1SC(C)(C)[C@@H]2C(=O)O ; Cc1onc(-c2ccccc2)c1C(=O)N[C@@H]1C(=O)N2[C@@H]1SC(C)(C)[C@@H]2C(=O)O ; Cc1onc(-c2ccccc2Cl)c1C(=O)N[C@@H]1C(=O)N2[C@@H]1SC(C)(C)[C@@H]2C(=O)O ; Cc1onc(-c2c(Cl)cccc2Cl)c1C(=O)N[C@@H]1C(=O)N2[C@@H]1SC(C)(C)[C@@H]2C(=O)O]

In some embodiments, a method of treating a disease comprises administering to a patient in need thereof an effective amount of a compound of Formula (VI), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (VI) is a TDO/IDO inhibitor, i.e. the compound of Formula (VI) is a TDO inhibitor, IDO inhibitor, or an inhibitor of both TDO and IDO. In some embodiments, the compound of Formula (VI) is not a TDO inhibitor. In some embodiments, a compound of Formula (VI) is not an IDO inhibitor. In some embodiments, a compound of Formula (VI) is not an inhibitor of both TDO and IDO. In some embodiments, the disease is cancer.

In another aspect, provided herein is a method of treating cancer comprising administering to a patient in need thereof an effective amount of a compound of Formula (VII), or a pharmaceutically acceptable salt or solvate thereof:

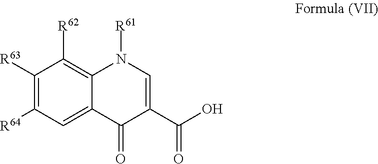

wherein
R⁶¹ is C₁-C₆ alkyl or cycloalkyl;
R⁶² is halogen, C₁-C₆ alkyl, or C₁-C₆ alkoxy; or
R⁶¹ and R⁶² taken together with the atoms to which they are attached form an optionally substituted heterocycloalkyl;
R⁶³ is optionally substituted cycloalkyl or optionally substituted heterocycloalkyl; and
R⁶⁴ is halogen, C₁-C₆ alkyl, or C₁-C₆ alkoxy.

In some embodiments, the compound of Formula (VII) or a pharmaceutically acceptable salt or solvate comprises:

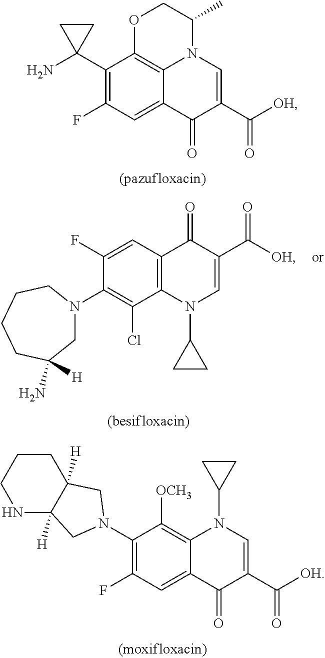 [SMILES: C[C@H]1COc2c(C3(N)CC3)c(F)cc3c(=O)c(C(=O)O)cn1c23 ; N[C@@H]1CCCCN(c2c(F)cc3c(=O)c(C(=O)O)cn(C4CC4)c3c2Cl)C1 ; COc1c(N2C[C@@H]3CCCN[C@@H]3C2)c(F)cc2c(=O)c(C(=O)O)cn(C3CC3)c12]

In some embodiments, a method of treating a disease comprises administering to a patient in need thereof an effective amount of a compound of Formula (VII), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (VII) is a TDO/IDO inhibitor, i.e. the compound of Formula (VII) is a TDO inhibitor, IDO inhibitor, or an inhibitor of both TDO and IDO. In some embodiments, the compound of Formula (VII) is not a TDO inhibitor. In some embodiments, a compound of Formula (VII) is not an IDO inhibitor. In some embodiments, a compound of Formula (VII) is not an inhibitor of both TDO and IDO. In some embodiments, the disease is cancer.

In another aspect, provided herein is a method of treating cancer comprising administering to a patient in need thereof an effective amount of a compound of Formula (VIII), or a pharmaceutically acceptable salt or solvate thereof:

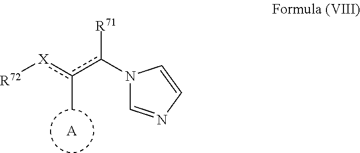

wherein
each [symbol] are independently a single bond or a double bond provided that they are not both double bonds;
Ring A is optionally substituted aryl, or optionally substituted heteroaryl;
X is O or S, when

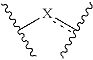

is

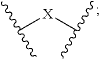

or
X is N, when

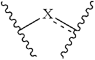

is

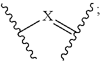

R⁷¹ is hydrogen or optionally substituted C₁-C₆ alkyl;
R⁷² is —(C₁-C₆ alkylene) (optionally substituted aryl), —(C₁-C₆ alkylene) (optionally substituted heteroaryl), —(C₁-C₆ alkylene)-O-(optionally substituted aryl), or —O—(C₁-C₆ alkylene) (optionally substituted aryl), provided that when R² is —O—(C₁-C₆ alkylene) (optionally substituted aryl), then X is N.

In some embodiments, the compound of Formula (VIII), or a pharmaceutically acceptable salt or solvate thereof is of Formula (VIIIa) or a pharmaceutically acceptable salt or solvate thereof:

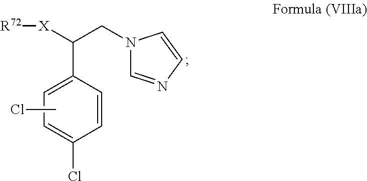

wherein
X is O or S; and
R⁷² is —(C₁-C₆ alkylene) (optionally substituted aryl) or —(C₁-C₆ alkylene) (optionally substituted heteroaryl).

In some embodiments, the compound of Formula (VIII) or a pharmaceutically acceptable salt or solvate comprises:

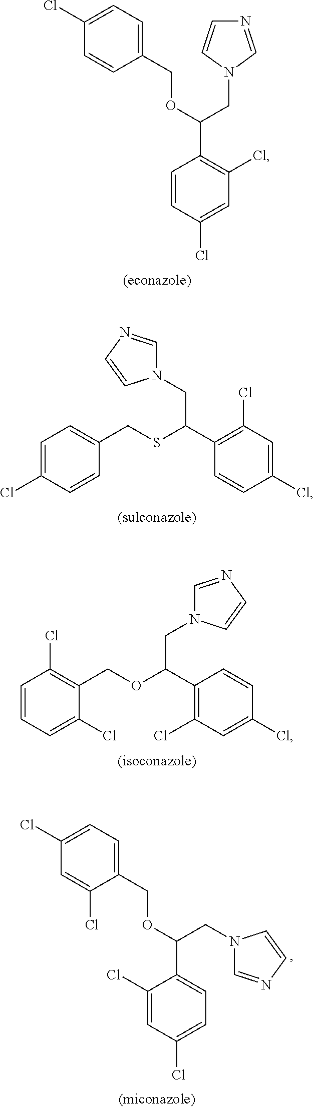 [SMILES: Clc1ccc(COC(Cn2ccnc2)c2ccc(Cl)cc2Cl)cc1 ; Clc1ccc(CSC(Cn2ccnc2)c2ccc(Cl)cc2Cl)cc1 ; Clc1ccc(C(Cn2ccnc2)OCc2c(Cl)cccc2Cl)c(Cl)c1 ; Clc1ccc(COC(Cn2ccnc2)c2ccc(Cl)cc2Cl)c(Cl)c1]

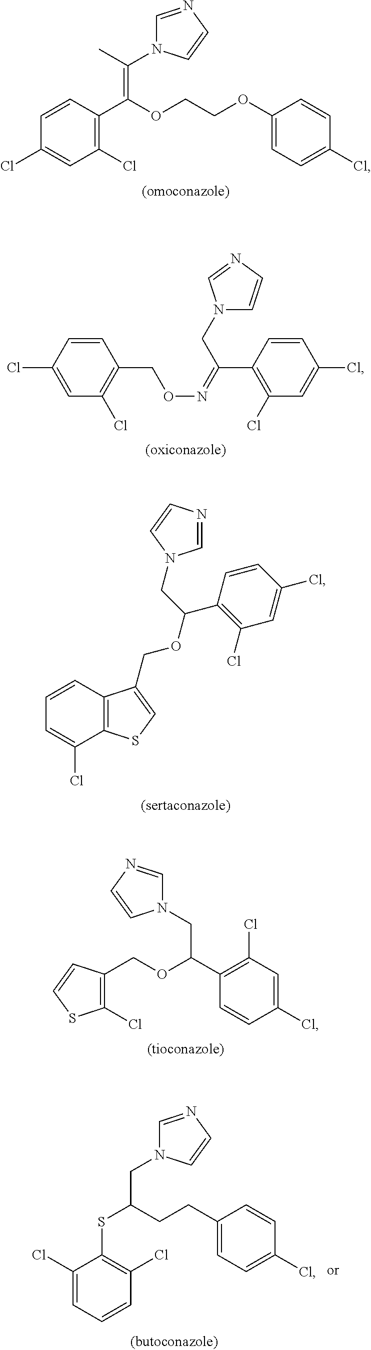 [SMILES: C/C(=C(/OCCOc1ccc(Cl)cc1)c1ccc(Cl)cc1Cl)n1ccnc1 ; Clc1ccc(CO/N=C(\Cn2ccnc2)c2ccc(Cl)cc2Cl)c(Cl)c1 ; Clc1ccc(C(Cn2ccnc2)OCc2csc3c(Cl)cccc23)c(Cl)c1 ; Clc1ccc(C(Cn2ccnc2)OCc2ccsc2Cl)c(Cl)c1 ; Clc1ccc(CCC(Cn2ccnc2)Sc2c(Cl)cccc2Cl)cc1 ; "or"]

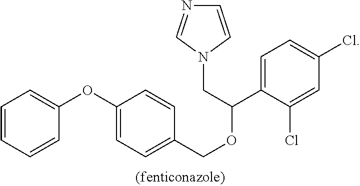 [SMILES: Clc1ccc(C(Cn2ccnc2)OCc2ccc(Oc3ccccc3)cc2)c(Cl)c1]

In some embodiments, a method of treating a disease comprises administering to a patient in need thereof an effective amount of a compound of Formula (VIII), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (VIII) is a TDO/IDO inhibitor, i.e. the compound of Formula (VIII) is a TDO inhibitor, IDO inhibitor, or an inhibitor of both TDO and IDO. In some embodiments, the compound of Formula (VIII) is not a TDO inhibitor. In some embodiments, a compound of Formula (VIII) is not an IDO inhibitor. In some embodiments, a compound of Formula (VIII) is not an inhibitor of both TDO and IDO. In some embodiments, the disease is cancer.

In one aspect, provided herein is a method of treating cancer comprising administering to a patient in need thereof an effective amount of a compound of Formula (IX), or a pharmaceutically acceptable salt or solvate thereof:

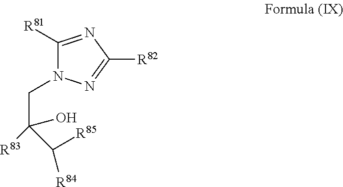

wherein
R⁸¹ is hydrogen, or optionally substituted C₁-C₆ alkyl;
R⁸² is hydrogen, or optionally substituted C₁-C₆ alkyl;
R⁸³ is optionally substituted aryl or optionally substituted heteroaryl;
R⁸⁴ is hydrogen, or optionally substituted C₁-C₆ alkyl;
or
the oxygen from the —OH and R⁸⁴ are taken together to form an epoxide; and
R⁸⁵ is optionally substituted aryl, optionally substituted heteroaryl, optionally substituted cycloalkyl, or optionally substituted heterocycloalkyl.

In some embodiments, R⁸¹ and R⁸² are both hydrogen. In some embodiments, R⁸³ is an optionally substituted phenyl. In some embodiments, R⁸³ is phenyl substituted with one or more halogen. In some embodiments, R⁸³ is phenyl substituted with one or more fluoro. In some embodiments, R⁸³ is phenyl substituted with two fluoro. In some embodiments, R⁸⁴ is hydrogen. In some embodiments, the oxygen from the —OH and R⁸⁴ are taken together to form an epoxide. In some embodiments, R⁸⁴ is methyl. In some embodiments, R⁸⁵ is optionally substituted aryl. In some embodiments, R⁸⁵ is optionally substituted heteroaryl. In some embodiments, R⁸⁵ is optionally substituted phenyl. In some embodiments, R⁸⁵ is optionally substituted thiazole, optionally substituted pyridine, optionally substituted pyrimidine, or optionally substituted triazole. In some embodiments, R⁸⁵ is optionally substituted heterocycloalkyl.

In some embodiments, the compound of Formula (IX) or a pharmaceutically acceptable salt or solvate comprises:

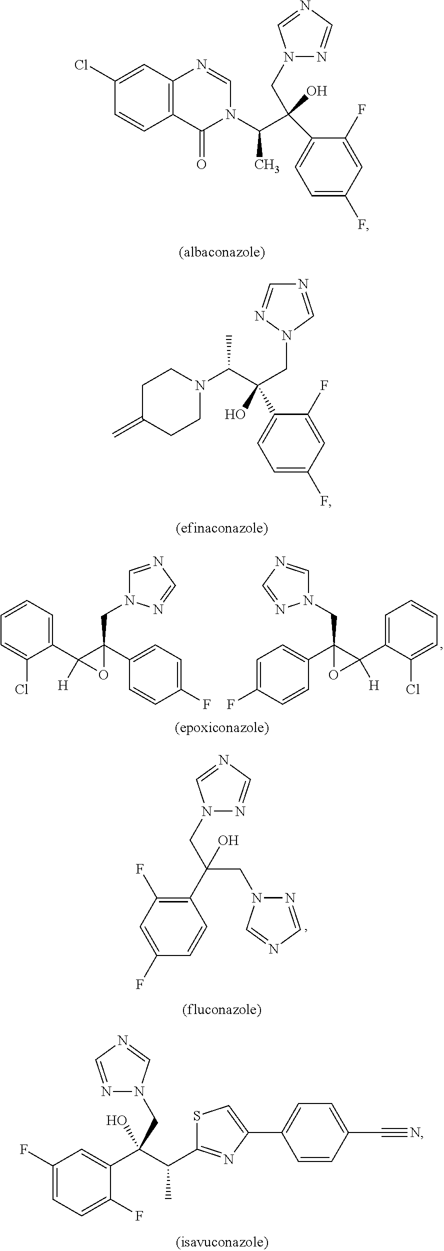

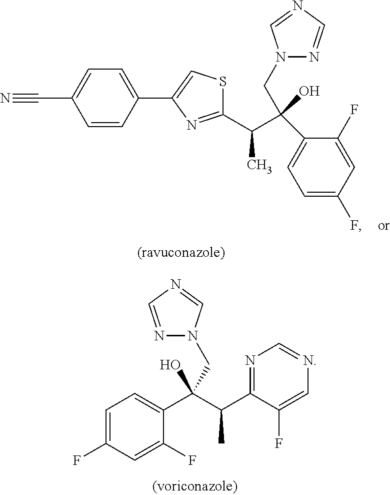 [SMILES: C[C@@H](c1nc(-c2ccc(C#N)cc2)cs1)[C@](O)(Cn1cncn1)c1ccc(F)cc1F ; C[C@H](c1ncncc1F)[C@@](O)(Cn1cncn1)c1ccc(F)cc1F]

In some embodiments, a method of treating a disease comprises administering to a patient in need thereof an effective amount of a compound of Formula (IX), or a salt or solvate thereof, and one or more additional active agents. Additional active agents include, without limitation, PD-1 inhibitors, IDO inhibitors, CTLA-4 inhibitors, TDO inhibitors, and combinations thereof. In some cases, an additional active agent is an anti-cancer agent. In some embodiments, the compound of Formula (IX) is a TDO/IDO inhibitor, i.e. the compound of Formula (IX) is a TDO inhibitor, IDO inhibitor, or an inhibitor of both TDO and IDO. In some embodiments, the compound of Formula (IX) is not a TDO inhibitor. In some embodiments, a compound of Formula (IX) is not an IDO inhibitor. In some embodiments, a compound of Formula (IX) is not an inhibitor of both TDO and IDO. In some embodiments, the disease is cancer.

In some embodiments, a method of treating a disease comprises administering to a patient in need thereof an effective amount of a compound of Formula (I), or a salt or solvate thereof, and an effective amount of a compound of Formula (II), a compound of Formula (III), a compound of Formula (IV), a compound of Formula (V), a compound of Formula (VI), a compound of Formula (VII), Formula (VIII), Formula (IX) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (I), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (II), or a salt or solvate thereof, and an effective amount of a compound of Formula (I), a compound of Formula (III), a compound of Formula (IV), a compound of Formula (V), a compound of Formula (VI), a compound of Formula (VII), Formula (VIII), Formula (IX) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (II), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (III), or a salt or solvate thereof, and an effective amount of a compound of Formula (II), a compound of Formula (I), a compound of Formula (IV), a compound of Formula (V), a compound of Formula (VI), a compound of Formula (VII), Formula (VIII), Formula (IX) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (III), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (IV), or a salt or solvate thereof, and an effective amount of a compound of Formula (II), a compound of Formula (III), a compound of Formula (I), a compound of Formula (V), a compound of Formula (VI), a compound of Formula (VII), Formula (VIII), Formula (IX) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (IV), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (V), or a salt or solvate thereof, and an effective amount of a compound of Formula (II), a compound of Formula (III), a compound of Formula (IV), a compound of Formula (I), a compound of Formula (VI), a compound of Formula (VII), Formula (VIII), Formula (IX) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (V), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (VI), or a salt or solvate thereof, and an effective amount of a compound of Formula (II), a compound of Formula (III), a compound of Formula (IV), a compound of Formula (V), a compound of Formula (I), a compound of Formula (VII), Formula (VIII), Formula (IX) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (VI), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (VII), or a salt or solvate thereof, and an effective amount of a compound of Formula (II), a compound of Formula (III), a compound of Formula (IV), a compound of Formula (V), a compound of Formula (VI), a compound of Formula (I), Formula (VIII), Formula (IX) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (VII), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (VIII), or a salt or solvate thereof, and an effective amount of a compound of Formula (II), a compound of Formula (III), a compound of Formula (IV), a compound of Formula (V), a compound of Formula (VI), a compound of Formula (I), Formula (VII), Formula (IX) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (IX), or a salt or solvate thereof, and an effective amount of a compound of Formula (II), a compound of Formula (III), a compound of Formula (IV), a compound of Formula (V), a compound of Formula (VI), a compound of Formula (I), Formula (VIII), Formula (VII) or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (VIII), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In some embodiments, a method of treating a disease comprises administering to a patient in need thereof an effective amount of a compound of Formula (IX), or a salt or solvate thereof, and an effective amount of an active agent comprising a compound of Table 1, or a salt or solvate of a compound of Table 1. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer.

In another aspect, provided herein is a method of treating cancer comprising administering to a patient in need thereof an effective amount of a compound or a pharmaceutically acceptable salt or solvate thereof comprising:

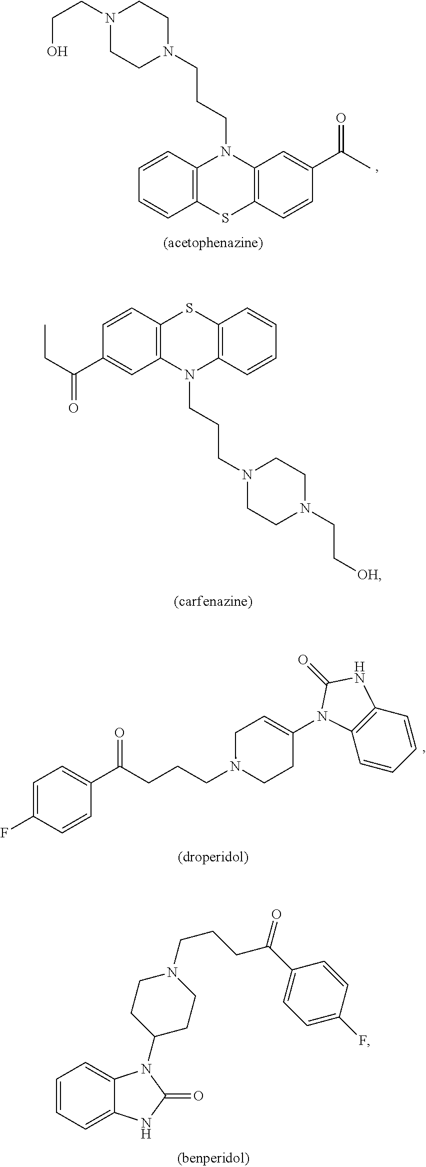 [SMILES: CC(=O)c1ccc2c(c1)N(CCCN1CCN(CCO)CC1)c1ccccc1S2 ; CCC(=O)c1ccc2c(c1)N(CCCN1CCN(CCO)CC1)c1ccccc1S2 ; O=C(CCCN1CC=C(n2c(=O)[nH]c3ccccc32)CC1)c1ccc(F)cc1 ; O=C(CCCN1CCC(n2c(=O)[nH]c3ccccc32)CC1)c1ccc(F)cc1]

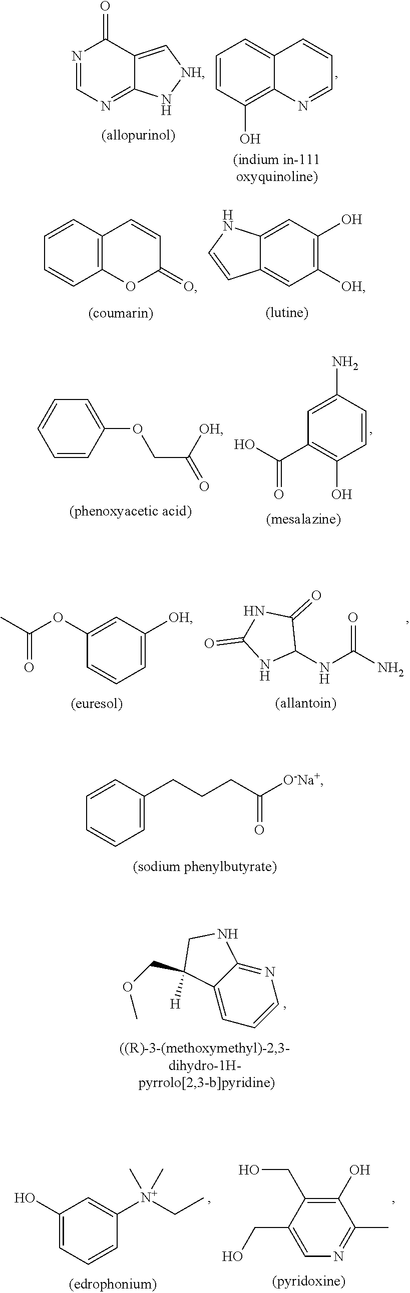

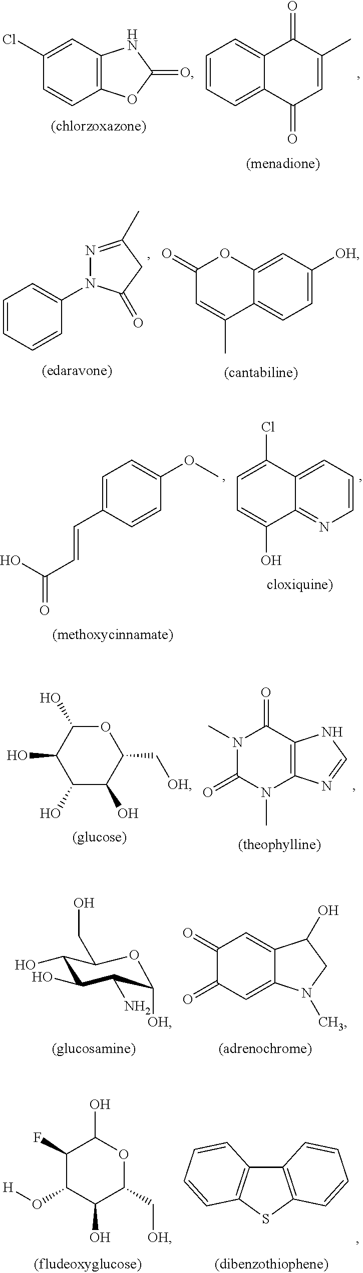

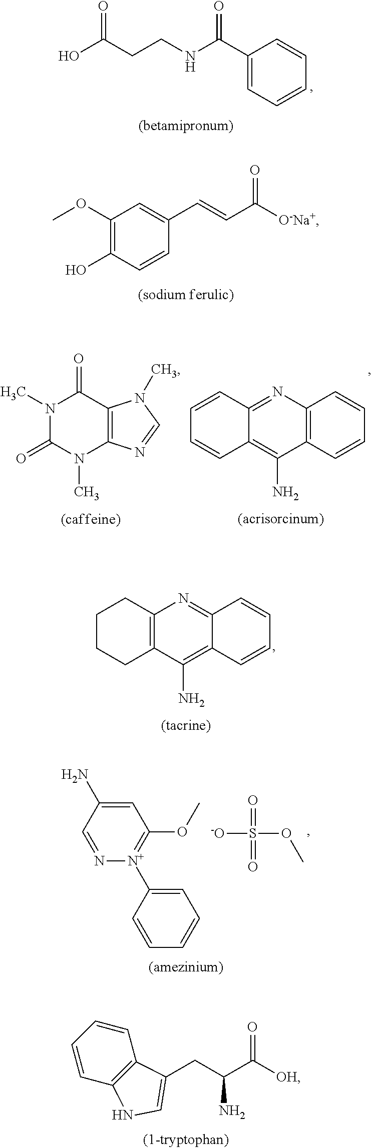

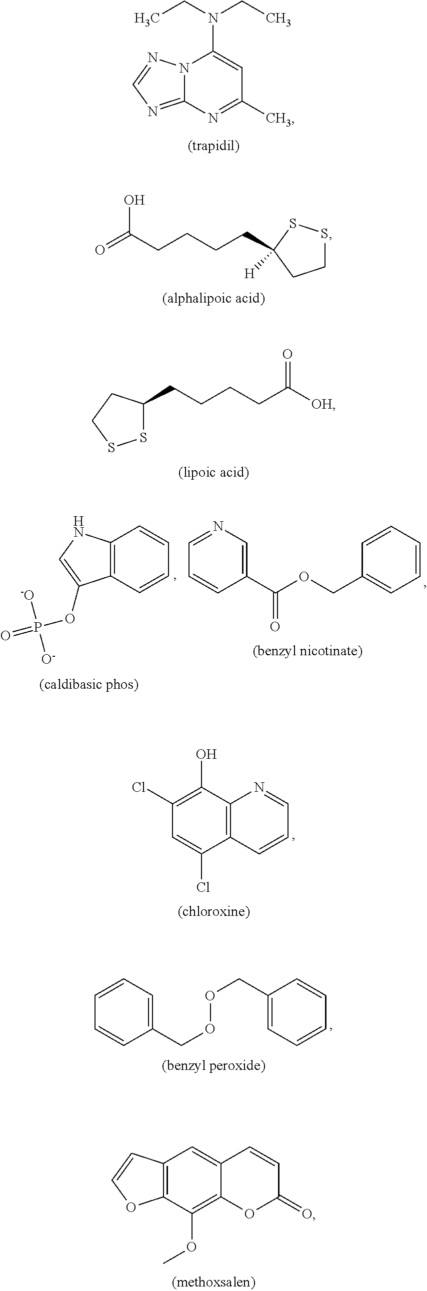

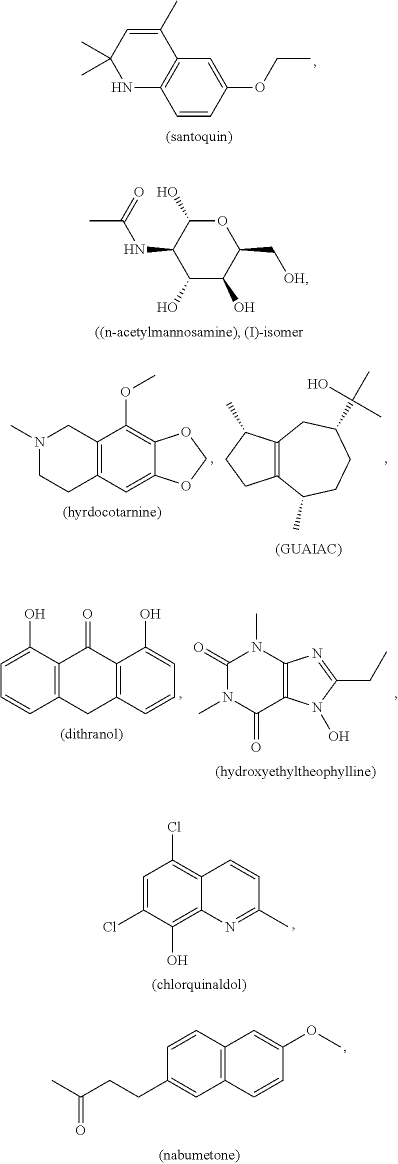

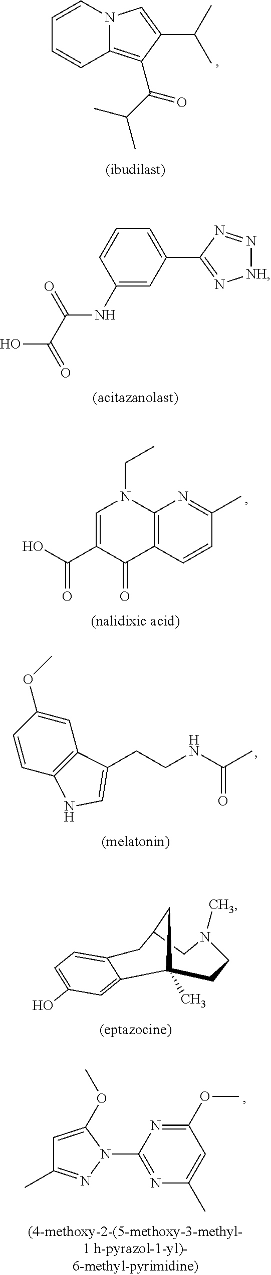

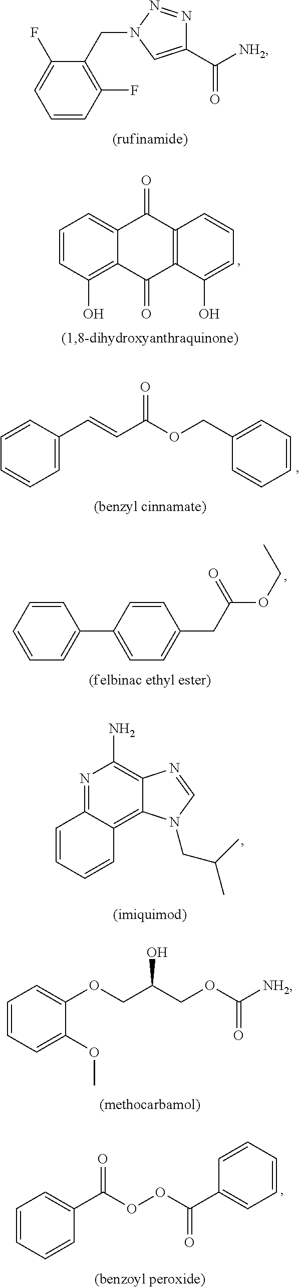

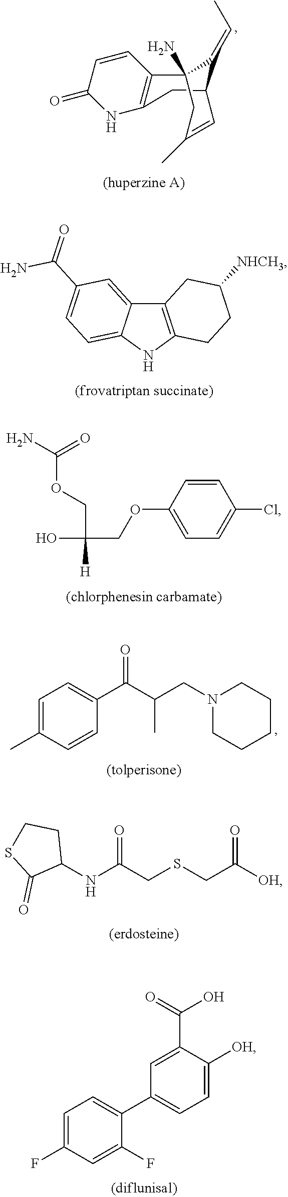 [SMILES: C/C=C1/[C@H]2C=C(C)C[C@]1(N)c1ccc(=O)[nH]c1C2 ; CN[C@@H]1CCc2[nH]c3ccc(C(N)=O)cc3c2C1 ; NC(=O)OC[C@@H](O)COc1ccc(Cl)cc1 ; Cc1ccc(C(=O)C(C)CN2CCCCC2)cc1 ; O=C(O)CSCC(=O)NC1CCSC1=O ; O=C(O)c1cc(-c2ccc(F)cc2F)ccc1O]

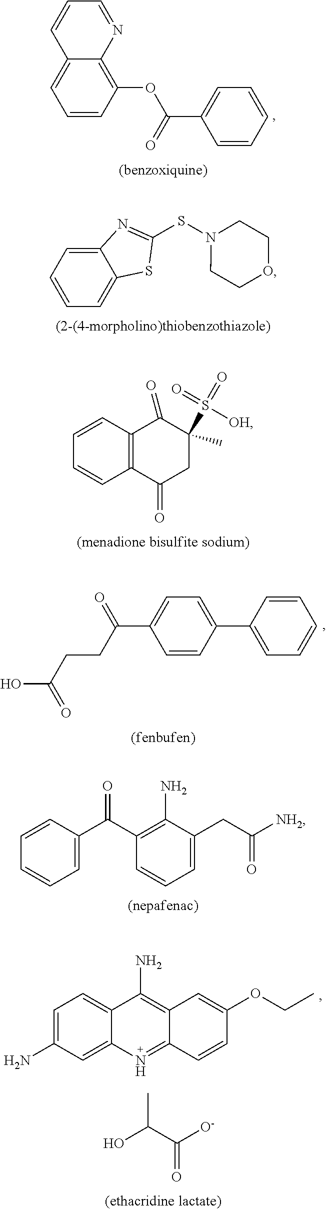

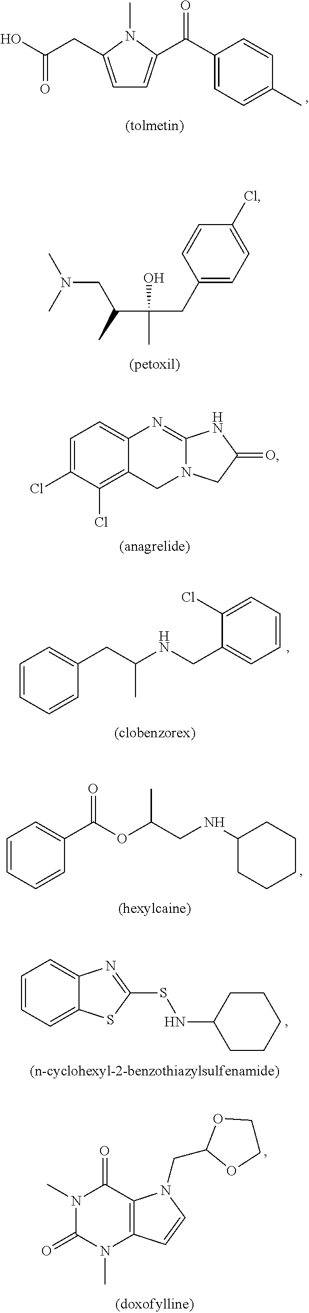

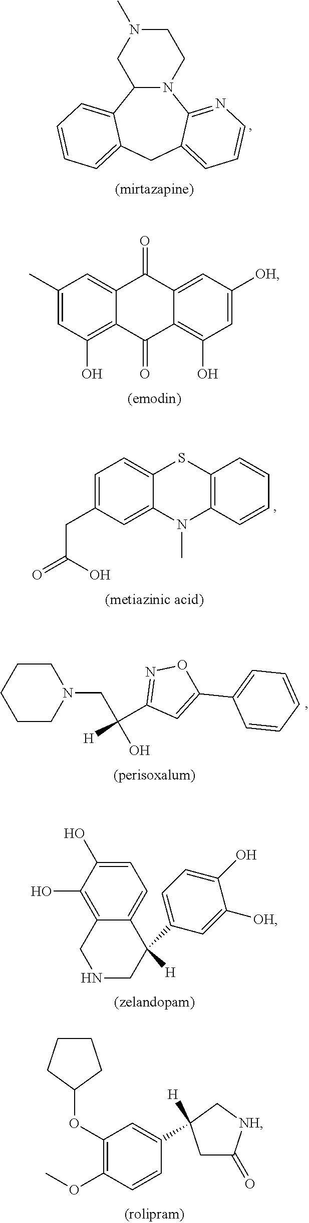 [SMILES: CN1CCN2c3ncccc3Cc3ccccc3C2C1 ; Cc1cc(O)c2c(c1)C(=O)c1cc(O)cc(O)c1C2=O ; CN1c2ccccc2Sc2ccc(CC(=O)O)cc21 ; O[C@H](CN1CCCCC1)c1cc(-c2ccccc2)on1 ; Oc1ccc([C@@H]2CNCc3c2ccc(O)c3O)cc1O ; COc1ccc([C@@H]2CNC(=O)C2)cc1OC1CCCC1]

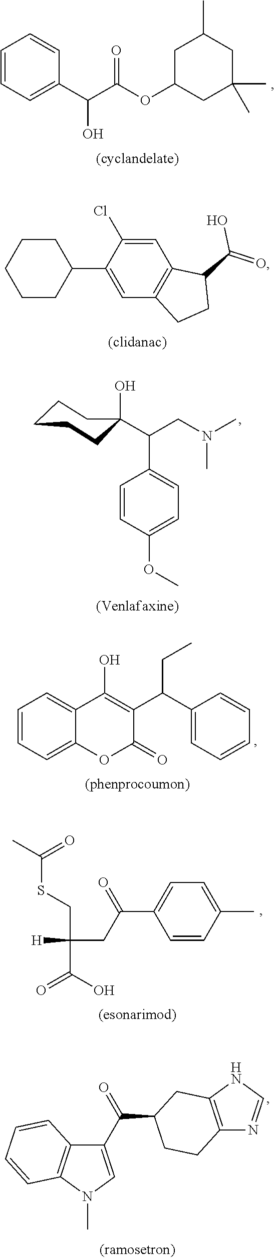

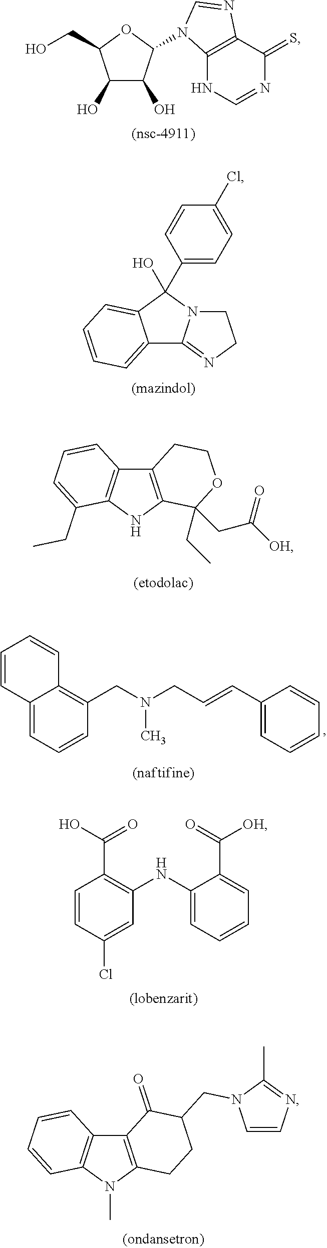 [SMILES: OC[C@H]1O[C@H](n2cnc3c(=S)nc[nH]c32)[C@@H](O)[C@H]1O ; OC1(c2ccc(Cl)cc2)c2ccccc2C2=NCCN21 ; CCc1cccc2c3c([nH]c12)C(CC)(CC(=O)O)OCC3 ; CN(C/C=C/c1ccccc1)Cc1cccc2ccccc12 ; O=C(O)c1ccccc1Nc1cc(Cl)ccc1C(=O)O ; Cc1nccn1CC1CCc2c(c3ccccc3n2C)C1=O]

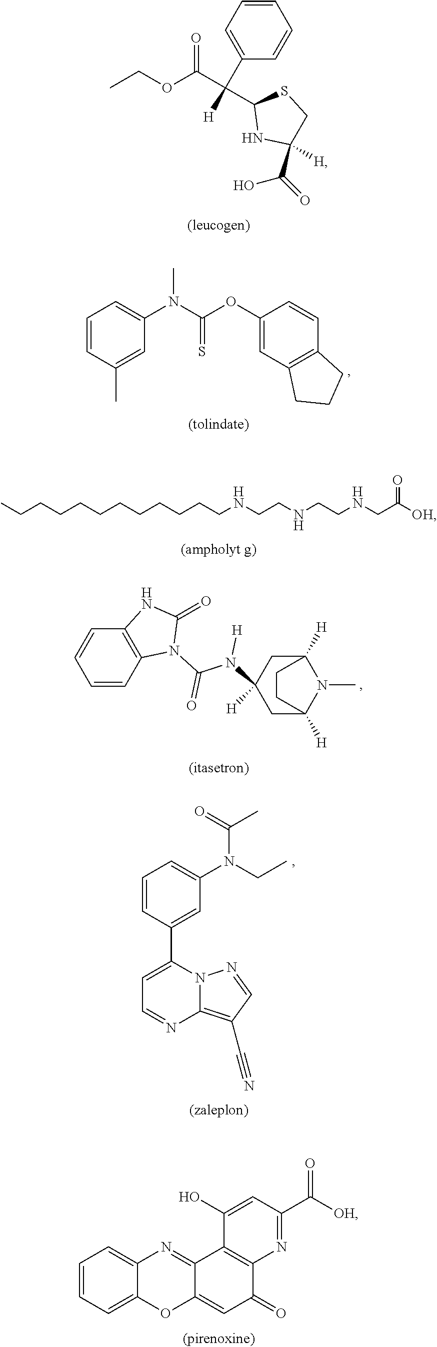

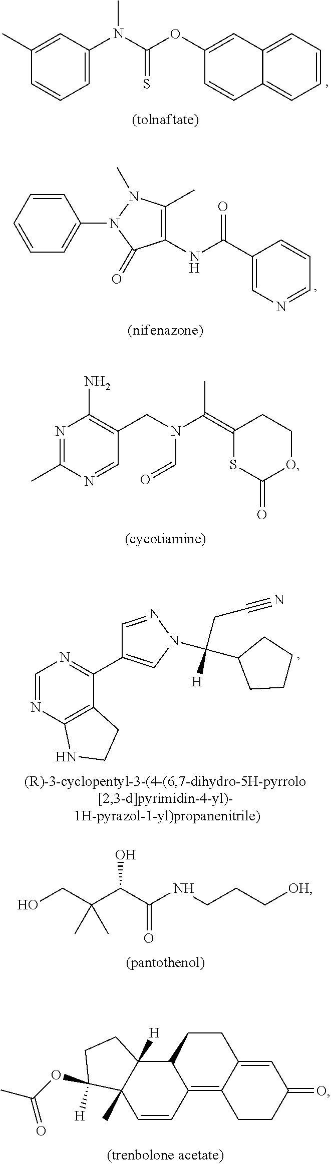

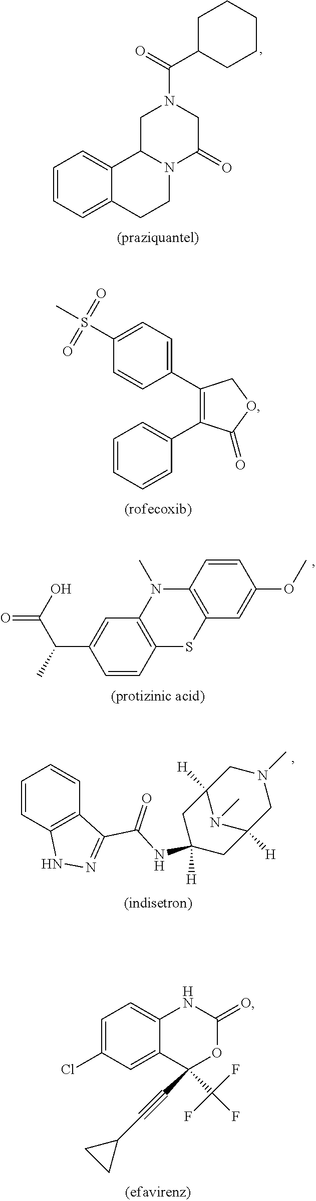 [SMILES: O=C(C1CCCCC1)N1CC(=O)N2CCc3ccccc3C2C1 ; CS(=O)(=O)c1ccc(C2=C(c3ccccc3)C(=O)OC2)cc1 ; COc1ccc2c(c1)Sc1ccc([C@H](C)C(=O)O)cc1N2C ; CN1C[C@H]2C[C@@H](NC(=O)c3n[nH]c4ccccc34)C[C@@H](C1)N2C ; O=C1Nc2ccc(Cl)cc2[C@@](C#CC2CC2)(C(F)(F)F)O1]

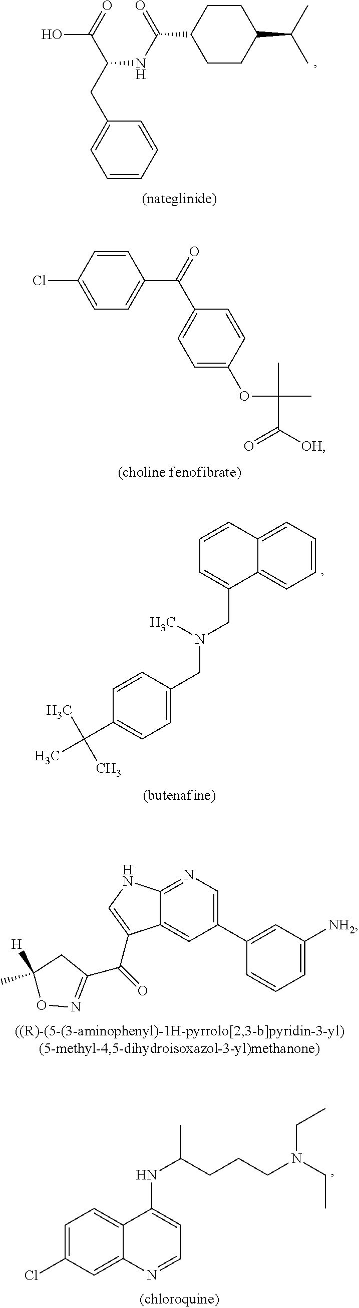

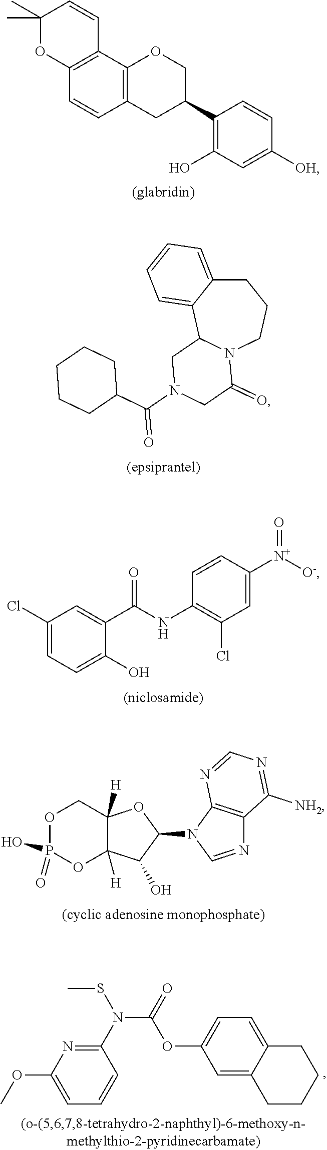

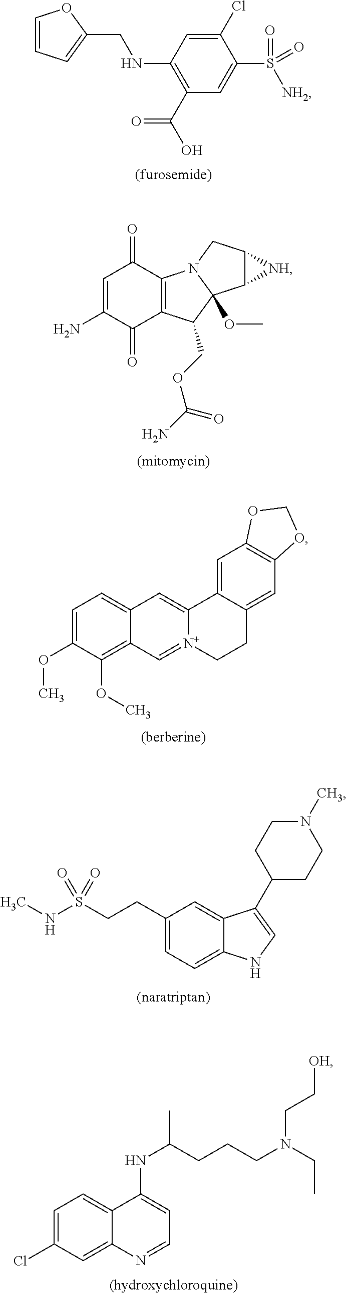 [SMILES: NS(=O)(=O)c1cc(C(=O)O)c(NCc2ccco2)cc1Cl ; CO[C@@]12[C@H](COC(N)=O)C3=C(C(=O)C=C(N)C3=O)N1C[C@@H]1N[C@@H]12 ; COc1ccc2cc3[n+](cc2c1OC)CCc1cc2c(cc1-3)OCO2 ; CNS(=O)(=O)CCc1ccc2[nH]cc(C3CCN(C)CC3)c2c1 ; CCN(CCO)CCCC(C)Nc1ccnc2cc(Cl)ccc12]

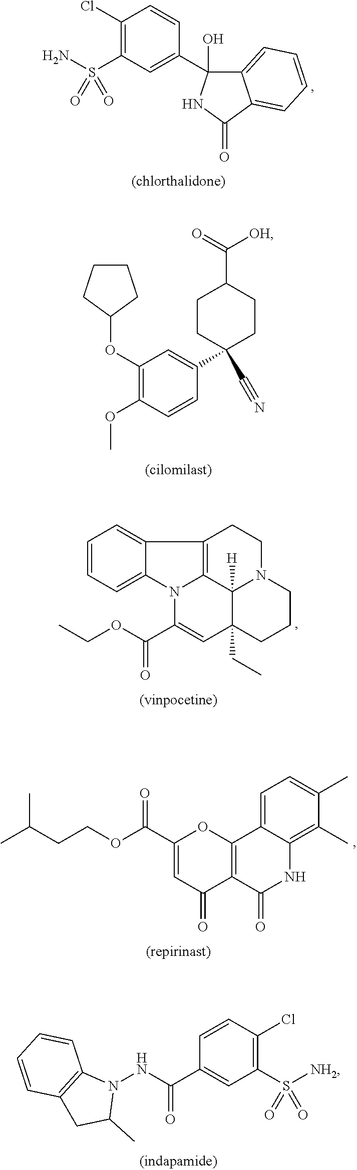

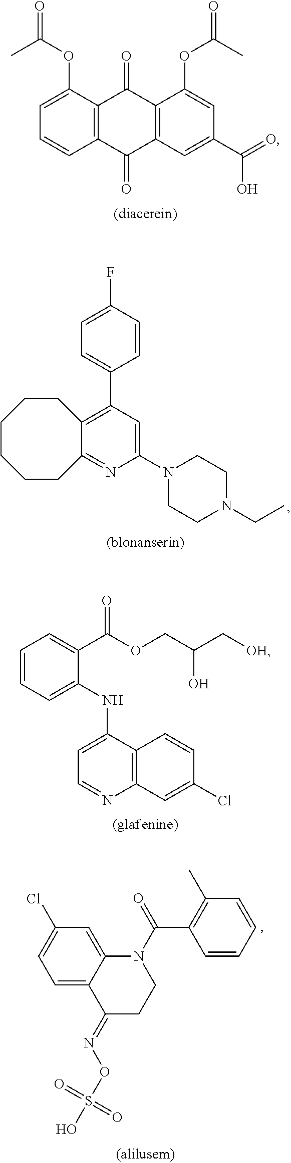 [SMILES: CC(=O)Oc1cccc2c1C(=O)c1c(OC(C)=O)cc(C(=O)O)cc1C2=O ; CCN1CCN(c2cc(-c3ccc(F)cc3)c3c(n2)CCCCCC3)CC1 ; O=C(OCC(O)CO)c1ccccc1Nc1ccnc2cc(Cl)ccc12 ; Cc1ccccc1C(=O)N1CC/C(=N\OS(=O)(=O)O)c2ccc(Cl)cc21]

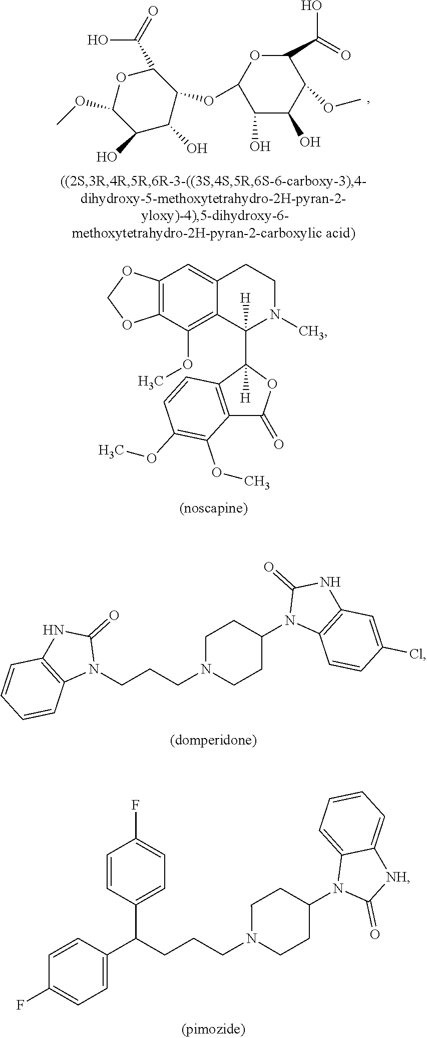

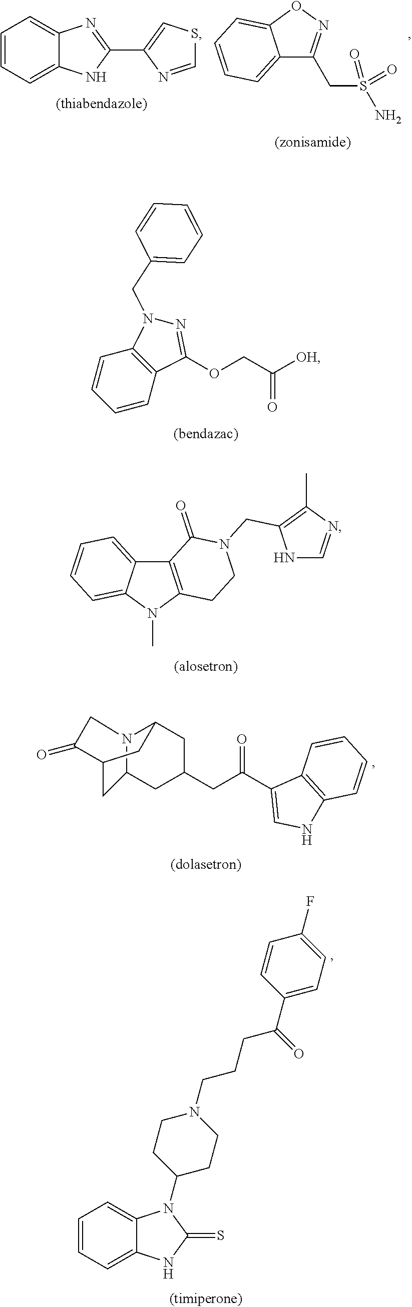

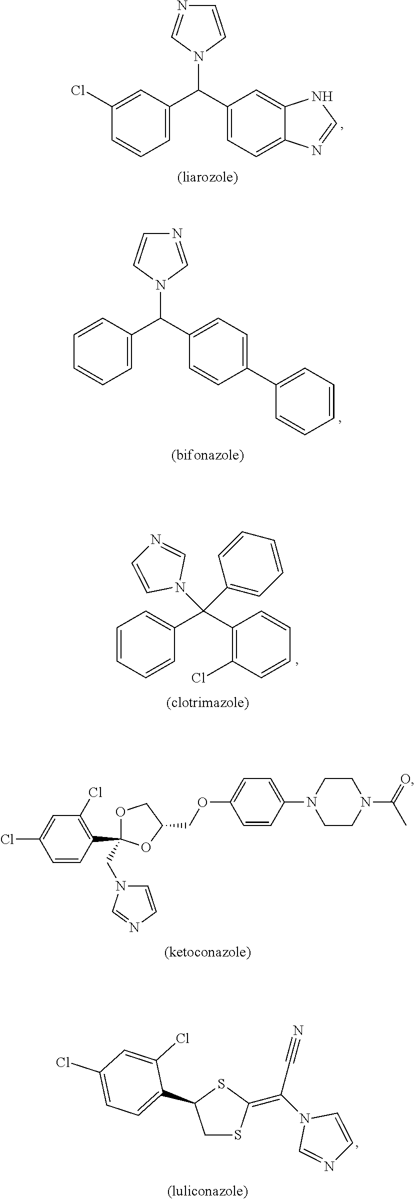 [SMILES: Clc1cccc(C(c2ccc3nc[nH]c3c2)n2ccnc2)c1 ; c1ccc(-c2ccc(C(c3ccccc3)n3ccnc3)cc2)cc1 ; Clc1ccccc1C(c1ccccc1)(c1ccccc1)n1ccnc1 ; CC(=O)N1CCN(c2ccc(OC[C@H]3CO[C@](Cn4ccnc4)(c4ccc(Cl)cc4Cl)O3)cc2)CC1 ; N#C/C(=C1/SC[C@@H](c2ccc(Cl)cc2Cl)S1)n1ccnc1]

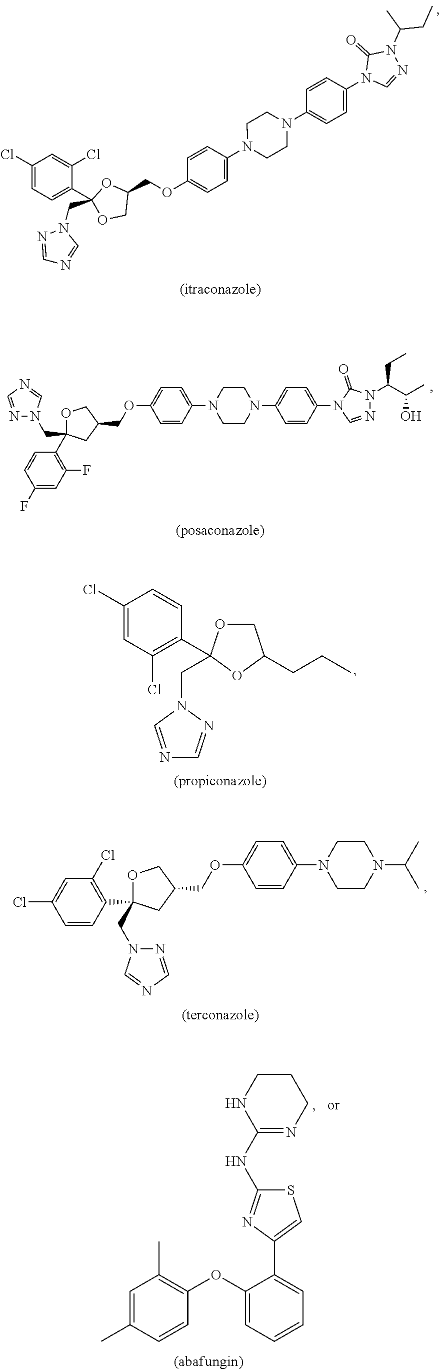

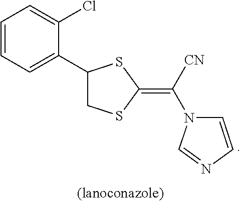 [SMILES: N#C/C(=C1/SCC(c2ccccc2Cl)S1)n1ccnc1]

In some embodiments, the method further comprises administering to the patient in need thereof an effective amount of a compound of Formula (I), a compound of Formula (II), a compound of Formula (III), a compound of Formula (IV), a compound of Formula (V), a compound of Formula (VI), a compound of Formula (VII), a compound of Formula (VIII), a compound of Formula (IX), or salts, solvates, or any combinations thereof. In some cases, the method further comprises administering to the patient one or more additional active agents comprising one or more PD-1 inhibitors, IDO inhibitors, CTLA-4 inhibitors, TDO inhibitors, anti-cancer agents, or combinations thereof. In some embodiments, the disease is cancer. In some embodiments, the method further comprises administering to the patient in need thereof an effective amount of an active agent comprising a compound of Table 1, or a salt or solvate of a compound of Table 1.

In some embodiments, a method of treating a disease or condition comprises the administration of an active agent comprising econazole, sulconazole, isoconazole, miconazole, sertaconazole, tioconazole, fenticonazole, liarozole, cloconazole, itraconazole, niclosamide, deferasirox, eltrombopag, or a pharmaceutically acceptable salt, solvate or combination thereof. In some instances, the disease is cancer. In some instances, the disease is an infectious disease. In some embodiments, the active agent is a TDO/IDO inhibitor, i.e. the active agent is a TDO inhibitor, IDO inhibitor, or an inhibitor of both TDO and IDO. In some embodiments, the active agent is not a TDO inhibitor. In some embodiments, an active agent is not an IDO inhibitor. In some embodiments, an active agent is not an inhibitor of both TDO and IDO. In some instances, the composition further comprises one or more additional active agents. In some instances, the composition is administered with one or more additional active agents. Additional active agents include, without limitation, PD-1 inhibitors (e.g., Table 2), IDO inhibitors (e.g., Table 1), CTLA-4 inhibitors (e.g., Table 3), and TDO inhibitors (e.g., Table 1). In some cases, an additional active agent is an anti-cancer agent.

### Combinations

In various aspects, provided herein are compositions comprising one or more TDO/IDO inhibitors (e.g., inhibitors of TDO, IDO, or both TDO and IDO) and one or more additional active agents useful for the treatment of a disease or condition. Further provided are compositions comprising one or more TDO/IDO inhibitors that are administered with compositions comprising one or more active agents for the treatment of a disease or condition. In some embodiments, one or more of the additional active agents are PD-1 inhibitors. In some embodiments, one or more of the additional active agents are IDO inhibitors. In some embodiments, one or more of the additional active agents are CTLA-4 inhibitors. In some embodiments, one or more of the additional active agents are TDO inhibitors. In some embodiments, one or more of the additional active agents are anti-cancer agents. In some embodiments, at least one TDO/IDO inhibitor is a small molecule. In some embodiments, at least one TDO/IDO inhibitor is a peptide. In some cases, at least one TDO/IDO inhibitor is not an immunoglobulin or immunoglobulin derived protein. Non-limiting examples of TDO/IDO inhibitors include the compounds of Table 1, Formula (I), Formula (II), Formula (III), Formula (IV), Formula (V), Formula (VI), Formula (VII), Formula (VIII), Formula (IX), and the salts and solvates thereof. TDO inhibitors include, without limitation, indoximod, F001287, NLG919, INCB024360, and any TDO inhibitor described elsewhere herein, such as in Table 1, and salts and solvates thereof. Non-limiting examples of PD-1 inhibitors include BMS-936559, MEDI4736, MPDL3280A, MSB0010718C, AMP-224, nivolumab, pembrolizumab, pidilizumab, BMS-986016, MDX1105-01, avelumab, any PD-1 inhibitor of Table 2, and salts and solvates thereof. Non-limiting examples of IDO inhibitors include indoximod, F001287, NLG919, INCB024360, any IDO inhibitors described elsewhere herein, such as in Table 1, and salts and solvates thereof. Non-limiting examples of CTLA-4 inhibitors include ipilimumab, tremelimumab, antibody clone 9H10, antibody clone BNI3, Del 60, M9-14 del 55, any CTLA-4 inhibitor of Table 3, and salts and solvates thereof.

In some instances, the disease is cancer and the treatment inhibits tumor cell survival. In some instances, the disease is cancer and the treatment promotes tumor cell death. In some instances, the disease is cancer and the treatment prevents tumor cell growth. In some instances, the disease is cancer and the treatment attenuates tumor cell growth. In some instances, the disease is cancer and the treatment prevents tumor cell invasion. In some instances, the disease is cancer and the treatment attenuates tumor cell invasion. In some instances, the disease is cancer and the treatment prevents tumor metastasis. In some instances, the disease is cancer and the treatment attenuates tumor metastasis. In some instances, the disease is an infectious disease, such as a disease caused by a bacteria, virus or parasite.

In some embodiments, a composition comprising one or more TDO/IDO inhibitors and one or more additional active agents are administered to a patient with one or more additional therapies. Additional therapies include, but are not limited to, chemotherapy and known cancer treatment methods.

In various aspects, provided herein are compositions comprising 2, 3, 4 or more TDO/IDO inhibitors. In some embodiments, at least one TDO/IDO inhibitor comprises a compound of Formula (I), or a salt or solvate thereof. In some embodiments, at least one TDO/IDO inhibitor comprises a compound of Formula (II), or a salt or solvate thereof. In some embodiments, at least one TDO/IDO inhibitor comprises a compound of Formula (III), or a salt or solvate thereof. In some embodiments, at least one TDO/IDO inhibitor comprises a compound of Formula (IV), or a salt or solvate thereof. In some embodiments, at least one TDO/IDO inhibitor comprises a compound of Formula (V), or a salt or solvate thereof. In some embodiments, at least one TDO/IDO inhibitor comprises a compound of Formula (VI), or a salt or solvate thereof. In some embodiments, at least one TDO/IDO inhibitor comprises a compound of Formula (VII), or a salt or solvate thereof. In some embodiments, at least one TDO/IDO inhibitor comprises a compound from Table 1, or a salt or solvate of a compound from Table 1. In some embodiments, the composition further comprises one or more PD-1 inhibitors, IDO inhibitors, CTLA-4 inhibitors, TDO inhibitors, anti-cancer agents, or any combination thereof.

Combinations with PD-1 Inhibitors

In some embodiments, a composition comprises one or more TDO/IDO inhibitors and one or more PD-1 inhibitors. In other embodiments, a composition comprising one or more TDO/IDO inhibitors is configured for administered with a composition comprising one or more PD-1 inhibitors. In some cases, a composition comprises 2, 3, 4, 5, or more TDO/IDO inhibitors. In some cases, a composition comprises 2, 3, 4, 5, or more PD-1 inhibitors. In some embodiments, a composition comprising one or more PD-1 inhibitors and one or more TDO/IDO inhibitors further comprises one or more IDO inhibitors, TDO and/or CTLA-4 inhibitors. In some embodiments, a composition comprising one or more TDO/IDO inhibitors and one or more PD-1 inhibitors is administered with one or more IDO inhibitors, TDO and/or CTLA-4 inhibitors. In some embodiments, a TDO/IDO inhibitor comprises a compound of Formula (I), Formula (II), Formula (III), Formula (IV), Formula (V), Formula (VI), Formula (VII), Formula (VIII), Formula (IX), Table 1, indoximod, F001287, NLG919, INCB024360, or a salt, solvate, or combination thereof. In some cases, a PD-1 inhibitor comprises BMS-936559, MEDI4736, MPDL3280A, MSB0010718C, AMP-224, nivolumab, pembrolizumab, pidilizumab, BMS-986016, MDX1105-01, avelumab, compound of Table 2, or a salt, solvate, or combination thereof.

In some embodiments, provided herein are compositions comprising an antimicrobial agent, a PD-1 inhibitor, and optionally one or more additional active agents. In some embodiments, provided herein are compositions comprising an antimicrobial agent administered with a PD-1 inhibitor and optionally one or more additional active agents. In some embodiments, the antimicrobial agent is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, the antimicrobial agent comprises an antibacterial agent. In some embodiments, the antibacterial agent comprises cloxiquine, betamipronum, chloroxine, nalidixic acid, pazufloxacin, propicillin, besifloxacin, oxacillin sodium, moxifloxacin, cloxacillin, dicloxacillin, or a salt, solvate or combination thereof. In some embodiments, the antimicrobial agent comprises an antifungal agent. In some embodiments, the antifungal agent comprises tioconazole, liarozole, sertaconazole, econazole, sulconazole, miconazole, isoconazole, itraconazole, fenticonazole, cloconazole, acrisorcinum, climbazole, croconazole, diphenylimidazole, exalamide, lanoconazole, oxiconazole, bifonazole, luliconazole, thiabendazole, butoconazole, clotrimazole, ketoconazole, luliconazole, albaconazole, efinaconazole, epoxiconazole, fluconazole, isavuconazole, posaconazole, propiconazole, ravuconazole, terconazole, voriconazole, abafungin, omoconazole, phenoxyacetic acid, naftifine, tolindate, tolnaftate, butenafine, o-(5,6,7,8-tetrahydro-2-naphthyl)-6-methoxy-n-methylthio-2-pyridinecarbamate, or a salt, solvate or combination thereof. In some embodiments, the antimicrobial agent comprises an antiparasitic agent. In some embodiments, the antiparasitic agent comprises praziquantel, chloroquine, epsiprantel, niclosamide, hydroxychloroquin, or a salt, solvate or combination thereof. In some embodiments, the antimicrobial agent comprises an antiseptic. In some embodiments, the antiseptic comprises iodochlorohydroxyquinoline, euresol, acrisorcinum, benzyl peroxide, benzoyl peroxide, benzoxiquine, ethacridine lactate, or a salt, solvate or combination thereof. In some embodiments, the antimicrobial agent comprises an antiviral agent. In some embodiments, the antiviral agent comprises imiquimod, efavirenz, or a salt, solvate or combination thereof. In some embodiments, the antimicrobial agent comprises chlorquinaldol, or a salt or solvate thereof. In some embodiments, a PD-1 inhibitor comprises an antimicrobial agent, sulfonamide, NSAID, ACE inhibitor, beta adrenergic agonist, beta antagonist, alpha adrenergic agonist, vitamin, benzamide, or a combination thereof. In some embodiments, a PD-1 inhibitor comprises a compound of Table 2, or a salt, solvate, or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, an IDO inhibitor comprises indoximod, F001287, NLG919, INCB024360, any IDO inhibitors described elsewhere herein, or a salt, solvate or combination thereof. In some embodiments, a CTLA-4 inhibitor comprises ipilimumab, tremelimumab, antibody clone 9H10, antibody clone BNI3, Del 60, M9-14 del 55, a compound of Table 3, or a salt, solvate or combination thereof.

In some embodiments, provided herein are compositions comprising one or more compounds of Table 1, or salts or solvates thereof; a PD-1 inhibitor, and optionally one or more additional active agents. In some embodiments, provided herein are compositions comprising one or more compounds of Table 1, or salts or solvates thereof, administered with a PD-1 inhibitor and optionally one or more additional active agents. In some embodiments, the compound of Table 1, or salt or solvate thereof, is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, a PD-1 inhibitor comprises an antimicrobial agent, sulfonamide, NSAID, ACE inhibitor, beta adrenergic agonist, beta antagonist, alpha adrenergic agonist, vitamin, benzamide, or a combination thereof. In some embodiments, a PD-1 inhibitor comprises a compound of Table 2, or a salt, solvate, or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, an IDO inhibitor comprises indoximod, F001287, NLG919, INCB024360, any IDO inhibitors described elsewhere herein, or a salt, solvate or combination thereof. In some embodiments, a CTLA-4 inhibitor comprises ipilimumab, tremelimumab, antibody clone 9H10, antibody clone BNI3, Del 60, M9-14 del 55, a compound of Table 3, or a salt, solvate or combination thereof.

In some embodiments, provided herein are compositions comprising a compound of Formula (I), Formula (II), Formula (III), Formula (IV), Formula (V), Formula (VI), Formula (VII), Formula (VIII), Formula (IX), or a salt or solvate thereof, a PD-1 inhibitor, and optionally one or more additional active agents. In some embodiments, provided herein are compositions comprising a compound of Formula (I), Formula (II), Formula (III), Formula (IV), Formula (V), Formula (VI), Formula (VII), Formula (VIII), Formula (IX), or a salt or solvate thereof, administered with a PD-1 inhibitor and optionally one or more additional active agents. In some embodiments, the compound of Formula (I), Formula (II), Formula (III), Formula (IV), Formula (V), Formula (VI), Formula (VII), Formula (VIII), Formula (IX), or salt or solvate thereof, is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, a PD-1 inhibitor comprises an antimicrobial agent, sulfonamide, NSAID, ACE inhibitor, beta adrenergic agonist, beta antagonist, alpha adrenergic agonist, vitamin, benzamide, or a combination thereof. In some embodiments, a PD-1 inhibitor comprises a compound of Table 2, or a salt, solvate, or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, an IDO inhibitor comprises indoximod, F001287, NLG919, INCB024360, any IDO inhibitors described elsewhere herein, or a salt, solvate or combination thereof. In some embodiments, a CTLA-4 inhibitor comprises ipilimumab, tremelimumab, antibody clone 9H10, antibody clone BNI3, Del 60, M9-14 del 55, a compound of Table 3, or a salt, solvate or combination thereof.

Combinations with IDO Inhibitors

In some embodiments, a composition comprises one or more TDO/IDO inhibitors (e.g., inhibitor of TDO, IDO, or both TDO and IDO) and one or more IDO inhibitors. In other embodiments, a composition comprising one or more TDO/IDO inhibitors is configured for administered with a composition comprising one or more IDO inhibitors. In some instances, for compositions comprising TDO/IDO inhibitors that further comprise and/or are administered with one or more IDO inhibitors, the compositions and/or administered combinations of inhibitors inhibit TDO, IDO, or both TDO and IDO to a greater extent that a TDO/IDO inhibitor or IDO inhibitor alone. Inhibition activity of compositions and combinations include in vitro and in vivo assays known to one of skill in the art. In some instances, an IDO inhibitor is a TDO inhibitor. In some cases, a composition comprises 2, 3, 4, 5, or more TDO/IDO inhibitors. In some cases, a composition comprises 2, 3, 4, 5, or more IDO inhibitors. In some embodiments, a composition comprising one or more IDO inhibitors and one or more TDO/IDO inhibitors further comprises one or more PD-1 inhibitors, TDO and/or CTLA-4 inhibitors. In some embodiments, a composition comprising one or more TDO/IDO inhibitors and one or more IDO inhibitors is administered with one or more PD-1 inhibitors, TDO and/or CTLA-4 inhibitors. In some embodiments, a TDO/IDO inhibitor comprises a compound of Formula (I), Formula (II), Formula (III), Formula (IV), Formula (V), Formula (VI), Formula (VII), Formula (VIII), Formula (IX), Table 1, indoximod, F001287, NLG919, INCB024360, or a salt, solvate, or combination thereof. In some cases, an IDO inhibitor comprises indoximod, F001287, NLG919, INCB024360, or a salt, solvate, or combination thereof. In some cases, an IDO inhibitor comprises a compound of Table 1, or a salt, solvate, or combination thereof. In some cases, any of the aforementioned IDO inhibitor compounds are also inhibitors of TDO.

In some embodiments, provided herein are compositions comprising an antimicrobial agent, an IDO inhibitor, and optionally one or more additional active agents. In some embodiments, provided herein are compositions comprising an antimicrobial agent administered with an IDO inhibitor and optionally one or more additional active agents. In some embodiments, the antimicrobial agent is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, the antimicrobial agent comprises an antibacterial agent. In some embodiments, the antibacterial agent comprises cloxiquine, betamipronum, chloroxine, nalidixic acid, pazufloxacin, propicillin, besifloxacin, oxacillin sodium, moxifloxacin, cloxacillin, dicloxacillin, or a salt, solvate or combination thereof. In some embodiments, the antimicrobial agent comprises an antifungal agent. In some embodiments, the antifungal agent comprises tioconazole, liarozole, sertaconazole, econazole, sulconazole, miconazole, isoconazole, itraconazole, fenticonazole, cloconazole, acrisorcinum, climbazole, croconazole, diphenylimidazole, exalamide, lanoconazole, oxiconazole, bifonazole, luliconazole, thiabendazole, butoconazole, clotrimazole, ketoconazole, luliconazole, albaconazole, efinaconazole, epoxiconazole, fluconazole, isavuconazole, posaconazole, propiconazole, ravuconazole, terconazole, voriconazole, abafungin, omoconazole, phenoxyacetic acid, naftifine, tolindate, tolnaftate, butenafine, o-(5,6,7,8-tetrahydro-2-naphthyl)-6-methoxy-n-methylthio-2-pyridinecarbamate, or a salt, solvate or combination thereof. In some embodiments, the antimicrobial agent comprises an antiparasitic agent. In some embodiments, the antiparasitic agent comprises praziquantel, chloroquine, epsiprantel, niclosamide, hydroxychloroquin, or a salt, solvate or combination thereof. In some embodiments, the antimicrobial agent comprises an antiseptic. In some embodiments, the antiseptic comprises iodochlorohydroxyquinoline, euresol, acrisorcinum, benzyl peroxide, benzoyl peroxide, benzoxiquine, ethacridine lactate, or a salt, solvate or combination thereof. In some embodiments, the antimicrobial agent comprises an antiviral agent. In some embodiments, the antiviral agent comprises imiquimod, efavirenz, or a salt, solvate or combination thereof. In some embodiments, the antimicrobial agent comprises chlorquinaldol, or a salt or solvate thereof. In some embodiments, an IDO inhibitor comprises an antimicrobial agent (e.g., antibacterial, antifungal, antiviral), antiprotozoal agent, taenicide, insecticide, sulfonamide (e.g., diuretic), NSAID, beta adrenergic agonist, beta adrenergic antagonist, alpha adrenergic agonist, sympathomimetic drug, antihistamine, GABA modulator, UV light absorber, or combination thereof. In some embodiments, an IDO inhibitor comprises a compound of Table 1, or a salt, solvate or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, a PD-1 inhibitor comprises BMS-936559, MEDI4736, MPDL3280A, MSB0010718C, AMP-224, nivolumab, pembrolizumab, pidilizumab, BMS-986016, MDX1105-01, avelumab, a compound of Table 2, or a salt, solvate or combination thereof. In some embodiments, a CTLA-4 inhibitor comprises ipilimumab, tremelimumab, antibody clone 9H10, antibody clone BNI3, Del 60, M9-14 del 55, a compound of Table 2, or a salt, solvate or combination thereof.

In some embodiments, provided herein are compositions comprising one or more compounds of Table 1, or salts or solvates thereof; an IDO inhibitor, and optionally one or more additional active agents. In some embodiments, provided herein are compositions comprising one or more compounds of Table 1, or salts or solvates thereof, administered with an IDO inhibitor and optionally one or more additional active agents. In some embodiments, the compound of Table 1, or salt or solvate thereof, is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, an IDO inhibitor comprises an antimicrobial agent (e.g., antibacterial, antifungal, antiviral), antiprotozoal agent, taenicide, insecticide, sulfonamide (e.g., diuretic), NSAID, beta adrenergic agonist, beta adrenergic antagonist, alpha adrenergic agonist, sympathomimetic drug, antihistamine, GABA modulator, UV light absorber, or combination thereof. In some embodiments, an IDO inhibitor comprises a compound of Table 1, or a salt, solvate or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, a PD-1 inhibitor comprises BMS-936559, MEDI4736, MPDL3280A, MSB0010718C, AMP-224, nivolumab, pembrolizumab, pidilizumab, BMS-986016, MDX1105-01, avelumab, a compound of Table 2, or a salt, solvate or combination thereof. In some embodiments, a CTLA-4 inhibitor comprises ipilimumab, tremelimumab, antibody clone 9H10, antibody clone BNI3, Del 60, M9-14 del 55, a compound of Table 3, or a salt, solvate or combination thereof.

In some embodiments, provided herein are compositions comprising a compound of Formula (I), Formula (II), Formula (III), Formula (IV), Formula (V), Formula (VI), Formula (VII), Formula (VIII), Formula (IX), or a salt or solvate thereof, an IDO inhibitor, and optionally one or more additional active agents. In some embodiments, provided herein are compositions comprising a compound of Formula (I), Formula (II), Formula (III), Formula (IV), Formula (V), Formula (VI), Formula (VII), Formula (VIII), Formula (IX), or a salt or solvate thereof, administered with an IDO inhibitor and optionally one or more additional active agents. In some embodiments, the compound of Formula (I), Formula (II), Formula (III), Formula (IV), Formula (V), Formula (VI), Formula (VII), Formula (VIII), Formula (IX), or salt or solvate thereof, is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, an IDO inhibitor comprises an antimicrobial agent (e.g., antibacterial, antifungal, antiviral), antiprotozoal agent, taenicide, insecticide, sulfonamide (e.g., diuretic), NSAID, beta adrenergic agonist, beta adrenergic antagonist, alpha adrenergic agonist, sympathomimetic drug, antihistamine, GABA modulator, UV light absorber, or combination thereof. In some embodiments, an IDO inhibitor comprises a compound of Table 1, or a salt, solvate or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, a PD-1 inhibitor comprises BMS-936559, MEDI4736, MPDL3280A, MSB0010718C, AMP-224, nivolumab, pembrolizumab, pidilizumab, BMS-986016, MDX1105-01, avelumab, a compound of Table 2, or a salt, solvate or combination thereof. In some embodiments, a CTLA-4 inhibitor comprises ipilimumab, tremelimumab, antibody clone 9H10, antibody clone BNI3, Del 60, M9-14 del 55, a compound of Table 3, or a salt, solvate or combination thereof.

Combinations with CTLA-4 Inhibitors

In some embodiments, a composition comprises one or more TDO/IDO inhibitors and one or more CTLA-4 inhibitors. In other embodiments, a composition comprising one or more TDO/IDO inhibitors is configured for administered with a composition comprising one or more CTLA-4 inhibitors. In some cases, a composition comprises 2, 3, 4, 5, or more TDO/IDO inhibitors. In some cases, a composition comprises 2, 3, 4, 5, or more CTLA-4 inhibitors. In some embodiments, a composition comprising one or more CTLA-4 inhibitors and one or more TDO/IDO inhibitors further comprises one or more IDO inhibitors, TDO and/or PD-1 inhibitors. In some embodiments, a composition comprising one or more TDO/IDO inhibitors and one or more CTLA-4 inhibitors is administered with one or more IDO inhibitors, TDO and/or PD-1 inhibitors. In some embodiments, a TDO/IDO inhibitor comprises a compound of Formula (I), Formula (II), Formula (III), Formula (IV), Formula (V), Formula (VI), Formula (VII), Formula (VIII), Formula (IX), Table 1, indoximod, F001287, NLG919, INCB024360, or a salt, solvate, or combination thereof. In some cases, a CTLA-4 inhibitor comprises ipilimumab, tremelimumab, antibody clone 9H10, antibody clone BNI3, Del 60, M9-14 del 55, or a combination thereof. In some cases, a CTLA-4 inhibitor comprises a compound of Table 3, or a salt, solvate or combination thereof.

In some embodiments, provided herein are compositions comprising an antimicrobial agent, a CTLA-4 inhibitor, and optionally one or more additional active agents. In some embodiments, provided herein are compositions comprising an antimicrobial agent administered with a CTLA-4 inhibitor and optionally one or more additional active agents. In some embodiments, the antimicrobial agent is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, the antimicrobial agent comprises an antibacterial agent. In some embodiments, the antibacterial agent comprises cloxiquine, betamipronum, chloroxine, nalidixic acid, pazufloxacin, propicillin, besifloxacin, oxacillin sodium, moxifloxacin, cloxacillin, dicloxacillin, or a salt, solvate or combination thereof. In some embodiments, the antimicrobial agent comprises an antifungal agent. In some embodiments, the antifungal agent comprises tioconazole, liarozole, sertaconazole, econazole, sulconazole, miconazole, isoconazole, itraconazole, fenticonazole, cloconazole, acrisorcinum, climbazole, croconazole, diphenylimidazole, exalamide, lanoconazole, oxiconazole, bifonazole, luliconazole, thiabendazole, butoconazole, clotrimazole, ketoconazole, luliconazole, albaconazole, efinaconazole, epoxiconazole, fluconazole, isavuconazole, posaconazole, propiconazole, ravuconazole, terconazole, voriconazole, abafungin, omoconazole, phenoxyacetic acid, naftifine, tolindate, tolnaftate, butenafine, o-(5,6,7,8-tetrahydro-2-naphthyl)-6-methoxy-n-methylthio-2-pyridinecarbamate, or a salt, solvate or combination thereof. In some embodiments, the antimicrobial agent comprises an antiparasitic agent. In some embodiments, the antiparasitic agent comprises praziquantel, chloroquine, epsiprantel, niclosamide, hydroxychloroquin, or a salt, solvate or combination thereof. In some embodiments, the antimicrobial agent comprises an antiseptic. In some embodiments, the antiseptic comprises iodochlorohydroxyquinoline, euresol, acrisorcinum, benzyl peroxide, benzoyl peroxide, benzoxiquine, ethacridine lactate, or a salt, solvate or combination thereof. In some embodiments, the antimicrobial agent comprises an antiviral agent. In some embodiments, the antiviral agent comprises imiquimod, efavirenz, or a salt, solvate or combination thereof. In some embodiments, the antimicrobial agent comprises chlorquinaldol, or a salt or solvate thereof. In some embodiments, a CTLA-4 inhibitor comprises an antibacterial agent, an antifungal agent, a sulfonamide, a NSAID, an antihistamine, a hormone, a serotonergic compound, a topoisomerase inhibitor, an alpha adrenergic antagonist, a phosphodiesterase inhibitor, a benzamide, a diazepine, or a combination thereof. In some embodiments, a CTLA-4 inhibitor comprises a compound of Table 3, or a salt, solvate, or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, an IDO inhibitor comprises indoximod, F001287, NLG919, INCB024360, a compound of Table 1, or a salt, solvate or combination thereof. In some embodiments, a PD-1 inhibitor comprises BMS-936559, MEDI4736, MPDL3280A, MSB0010718C, AMP-224, nivolumab, pembrolizumab, pidilizumab, BMS-986016, MDX1105-01, avelumab, a compound of Table 1, or a salt, solvate or combination thereof.

In some embodiments, provided herein are compositions comprising one or more compounds of Table 1, or salts or solvates thereof; a CTLA-4 inhibitor, and optionally one or more additional active agents. In some embodiments, provided herein are compositions comprising one or more compounds of Table 1, or salts or solvates thereof, administered with a CTLA-4 inhibitor and optionally one or more additional active agents. In some embodiments, the compound of Table 1, or salt or solvate thereof, is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, a CTLA-4 inhibitor comprises an antibacterial agent, an antifungal agent, a sulfonamide, a NSAID, an antihistamine, a hormone, a serotonergic compound, a topoisomerase inhibitor, an alpha adrenergic antagonist, a phosphodiesterase inhibitor, a benzamide, a diazepine, or a combination thereof. In some embodiments, a CTLA-4 inhibitor comprises ipilimumab, tremelimumab, antibody clone 9H10, antibody clone BNI3, Del 60, M9-14 del 55, a compound of Table 3, or a salt, solvate, or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, an IDO inhibitor comprises indoximod, F001287, NLG919, INCB024360, a compound of Table 1, or a salt, solvate or combination thereof. In some embodiments, a PD-1 inhibitor comprises BMS-936559, MEDI4736, MPDL3280A, MSB0010718C, AMP-224, nivolumab, pembrolizumab, pidilizumab, BMS-986016, MDX1105-01, avelumab, a compound of Table 2, or a salt, solvate or combination thereof.

In some embodiments, provided herein are compositions comprising a compound of Formula (I), Formula (II), Formula (III), Formula (IV), Formula (V), Formula (VI), Formula (VII), Formula (VIII), Formula (IX), or a salt or solvate thereof, a CTLA-4 inhibitor, and optionally one or more additional active agents. In some embodiments, provided herein are compositions comprising a compound of Formula (I), Formula (II), Formula (III), Formula (IV), Formula (V), Formula (VI), Formula (VII), Formula (VIII), Formula (IX), or a salt or solvate thereof, administered with a CTLA-4 inhibitor and optionally one or more additional active agents. In some embodiments, the compound of Formula (I), Formula (II), Formula (III), Formula (IV), Formula (V), Formula (VI), Formula (VII), Formula (VIII), Formula (IX), or salt or solvate thereof, is a TDO/IDO inhibitor. In various embodiments, the compositions are useful for the treatment of a disease or condition. In some cases, the disease is cancer. In some cases, the disease is an infectious disease. In some embodiments, an additional active agent comprises an anti-cancer agent, CTLA-4 inhibitor, PD-1 inhibitor, IDO inhibitor, TDO inhibitor, or combinations thereof. In some embodiments, a CTLA-4 inhibitor comprises an antibacterial agent, an antifungal agent, a sulfonamide, a NSAID, an antihistamine, a hormone, a serotonergic compound, a topoisomerase inhibitor, an alpha adrenergic antagonist, a phosphodiesterase inhibitor, a benzamide, a diazepine, or a combination thereof. In some embodiments, a CTLA-4 inhibitor comprises ipilimumab, tremelimumab, antibody clone 9H10, antibody clone BNI3, Del 60, M9-14 del 55, compound of Table 3, or a salt, solvate, or combination thereof. In some embodiments, a TDO/IDO inhibitor comprises a compound of Table 1, Formula (I), Formula (II), Formula (III), Formula (IV), Formula (V), Formula (VI), Formula (VII), Formula (VIII), Formula (IX), indoximod, F001287, NLG919, INCB024360, any TDO/IDO inhibitor described elsewhere herein, or a salt, solvate, or combination thereof. In some embodiments, an IDO inhibitor comprises indoximod, F001287, NLG919, INCB024360, a compound of Table 1, or a salt, solvate or combination thereof. In some embodiments, a PD-1 inhibitor comprises BMS-936559, MEDI4736, MPDL3280A, MSB0010718C, AMP-224, nivolumab, pembrolizumab, pidilizumab, BMS-986016, MDX1105-01, avelumab, a compound of Table 2, or a salt, solvate or combination thereof.

### Pharmaceutical Compositions

In some embodiments, disclosed herein are compositions comprising one or more TDO/IDO inhibitors (e.g., inhibitors of TDO, IDO, or both TDO and IDO), wherein the compositions are formulated for administration to a patient in need thereof. In some embodiments, disclosed herein are compositions comprising one or more TDO/IDO inhibitors and one or more additional active agents, wherein the compositions are formulated for administration to a patient in need thereof. In some cases, an additional active agent is a PD-1 inhibitor. In some cases, an additional active agent is an IDO inhibitor. In some cases, an additional active agent is an CTLA-4 inhibitor. In some cases, an additional active agent is a TDO inhibitor. In some cases, the additional active agent is an anti-cancer agent. In some cases, the patient has a disease or condition such as cancer and/or an infectious disease.

Further provided herein are pharmaceutical combinations that result from the mixing or combining of more than one active agents or ingredients. A pharmaceutical combination includes both fixed and non-fixed combinations of the active agents. In some cases, the active agents are provided in a fixed combination, where the active agents of the fixed combination are administered to a patient simultaneously in the form of a single entity or dosage. In some cases, the active agents are provided in a non-fixed combination, wherein the active agents of the non-fixed combination are administered to a patient as separate entities either simultaneously, concurrently or sequentially with no specific intervening time limits, and wherein such administration provides effective levels of the two compounds in the body of the patient. The latter also applies to cocktail therapy, for example, the administration of three or more active agents.

As used herein, a TDO/IDO inhibitor includes its pharmaceutically acceptable salts and/or solvates. Similarly, reference to an active agent includes its pharmaceutically acceptable salts and/or solvates. Further, reference to a composition comprising a TDO/IDO inhibitor is inclusive of a composition comprising a pharmaceutically acceptable salt and/or solvate of the TDO/IDO inhibitor. In some embodiments, one or more compounds of Table 1, and salts and solvates thereof, are TDO/IDO inhibitors.

A pharmaceutical composition or composition, in various embodiments, comprises an active agent as described herein. Non-limiting examples of active agents include anti-cancer agents, PD-1 inhibitors, IDO inhibitors, CTLA-4 inhibitors, TDO/IDO inhibitors, TDO inhibitors, and combinations thereof. In many embodiments, a composition is suitable for administration to a subject, such as a human. In some embodiments, the composition is sterile and preferably free of contaminants that are capable of eliciting an undesirable response within a subject. In some embodiments, the composition is of pharmaceutical grade. Pharmaceutical compositions are designed for administration to a patient in need thereof via a number of non-limiting routes including, without limitation, oral, intravenous, buccal, rectal, parenteral, intraperitoneal, intradermal, intracheal, intramuscular, subcutaneous, gastric or duodenal feeding tube, and inhalational. The terms administer, administering, administration, and the like, as used herein, refer to the methods that may be used to enable delivery of compounds or compositions to the desired site of biological action. These methods include, but are not limited to oral routes, inhalational, transdermal, transmucosal, sublingual, buccal, intraduodenal routes, parenteral injection (including intravenous, subcutaneous, intraperitoneal, intramuscular, intravascular, intraarterial, intracardial, intradermal, intraduodenal, intramedullary, intraosseous, intrathecal, intravitreal, epidural or infusion), topical (including epicutaneous, dermal, enema, eye drops, ear drops, intranasal, vaginal) and rectal administration. In some embodiments, the compounds and compositions described herein are administered orally. The terms co-administration or the like, as used herein, encompass administration of the selected therapeutic agents to a single patient, and include treatment regimens in which the agents are administered by the same or different route of administration, at the same or different time. In some embodiments, the compositions described herein are administered either alone or in combination with pharmaceutically acceptable carriers, excipients or diluents in a pharmaceutical composition.

In some embodiments, pharmaceutical compositions suitable for oral administration are presented as discrete units such as capsules, cachets or tablets each containing a predetermined amount of an active agent (e.g., TDO/IDO inhibitor); as a powder or granules; as a solution or a suspension in an aqueous liquid or a non-aqueous liquid; or as an oil-in-water liquid emulsion or a water-in-oil liquid emulsion. In some embodiments, the active agent is presented as a bolus, electuary or paste.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, optionally with an added preservative. The compositions may take such forms as suspensions, solutions or emulsions in oily or aqueous vehicles, and may contain formulatory agents such as suspending, stabilizing and/or dispersing agents. In some embodiments, the compositions are formulated in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Pharmaceutical compositions for parenteral administration include aqueous and non-aqueous (oily) sterile injection solutions of the active agents which may contain antioxidants, buffers, bacteriostats and solutes which render the formulation isotonic with the blood of the intended recipient; and aqueous and non-aqueous sterile suspensions which may include suspending agents and thickening agents.

In some embodiments, pharmaceutical compositions are formulated as a depot preparation, for example, for administration by implantation (e.g., subcutaneously or intramuscularly) or by intramuscular injection. In some cases, the compositions are formulated with suitable polymeric or hydrophobic materials or ion exchange resins, or as sparingly soluble derivatives, for example, as a sparingly soluble salt.

In some embodiments, pharmaceutical compositions are formed into tablets, lozenges, pastilles or gels for buccal or sublingual administration.

In some embodiments, pharmaceutical compositions are formulated in rectal compositions such as suppositories or retention enemas, e.g., containing conventional suppository bases such as cocoa butter, polyethylene glycol, or other glycerides.

In some embodiments, pharmaceutical compositions are administered topically such that the compound does not significantly enter the blood stream. Pharmaceutical compositions suitable for topical administration include liquid or semi-liquid preparations suitable for penetration through the skin such as gels, liniments, lotions, creams, ointments or pastes, and drops suitable for administration to the eye, ear or nose.

In some embodiments, pharmaceutical compositions are formulated for administration by inhalation using an insufflator, nebulizer pressurized packs or other convenient means of delivering an aerosol spray. Pressurized packs include those comprising a suitable propellant such as dichlorodifluoromethane, trichlorofluoromethane, dichlorotetrafluoroethane, carbon dioxide or other suitable gas. In some cases, pharmaceutical compositions are formulated as a dry powder for administered with the aid of an inhalator or insufflator.

In various aspects, one or more active agents of compositions described herein (e.g., PD-1 inhibitors, IDO inhibitors, CTLA-4 inhibitors, TDO/IDO inhibitors, TDO inhibitors, anti-cancer agents, and combinations thereof) are in the form of pharmaceutically acceptable salts. In additional embodiments, one or more active agents exist in unsolvated or solvated forms with pharmaceutically acceptable solvents such as water, ethanol, and the like. Reference to a PD-1 inhibitor herein includes both pharmaceutically acceptable salts and solvates of the PD-1 inhibitor. Reference to an IDO inhibitor herein includes both pharmaceutically acceptable salts and solvates of the IDO inhibitor. Reference to a CTLA-4 inhibitor herein includes both pharmaceutically acceptable salts and solvates of the CTLA-4 inhibitor. Reference to a TDO/IDO inhibitor herein includes both pharmaceutically acceptable salts and solvates of the TDO/IDO inhibitor. Reference to a TDO inhibitor herein includes both pharmaceutically acceptable salts and solvates of the TDO inhibitor. In some embodiments, pharmaceutically acceptable refers a material, such as a carrier or diluent, which does not abrogate the biological activity or properties of the agent, and is relatively nontoxic, i.e., the material is administered to a subject without causing undesirable biological effects or interacting in a deleterious manner with any of the agents of the composition in which it is contained.

In some embodiments, a pharmaceutically acceptable salt refers to a form of a therapeutically active agent that consists of a cationic form of the therapeutically active agent in combination with a suitable anion, or in alternative embodiments, an anionic form of the therapeutically active agent in combination with a suitable cation. In some embodiments, pharmaceutically acceptable salts are obtained by reacting an active agent (e.g., TDO/IDO inhibitor) with an acid. In some embodiments, the active agent is basic and is reacted with an organic acid or an inorganic acid. In some embodiments, an active agent is prepared as a chloride salt, sulfate salt, bromide salt, mesylate salt, maleate salt, citrate salt or phosphate salt. In some embodiments, an active agent is prepared as a hydrochloride salt. In some embodiments, pharmaceutically acceptable salts are obtained by reacting an active agent with a base. In some embodiments, the active agent is acidic and is reacted with a base. In some embodiments, the active agent is prepared as a sodium salt, calcium salt, potassium salt, magnesium salt, meglumine salt, N-methylglucamine salt or ammonium salt. In some embodiments, the compounds provided herein are prepared as a sodium salt.

In some embodiments, compositions described herein comprise one or more active agents that are prepared as prodrugs. A prodrug refers to an agent that is converted into the parent drug in vivo. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound. In some aspects, provided are compositions comprising a prodrug formulation of a CTLA-4 inhibitor described herein. In some aspects, provided are compositions comprising a prodrug formulation of an IDO inhibitor described herein. In some aspects, provided are compositions comprising a prodrug formulation of a PD-1 inhibitor described herein. In some aspects, provided are compositions comprising a prodrug formulation of a TDO/IDO inhibitor described herein. In some aspects, provided are compositions comprising a prodrug formulation of a TDO inhibitor described herein. In additional embodiments, provided are compositions comprising a prodrug formulation of a TDO/IDO inhibitor described herein and at least one additional active agent, for example, a PD-1 inhibitor, IDO inhibitor, TDO inhibitor, and/or CTLA-4 inhibitor. In additional embodiments, provided are compositions comprising a prodrug formulation of a TDO/IDO inhibitor described herein and a prodrug formulation of an active agent, for example, a PD-1 inhibitor, IDO inhibitor, TDO inhibitor, and/or CTLA-4 inhibitor. In additional embodiments, provided are compositions comprising a TDO/IDO inhibitor described herein and a prodrug formulation of an active agent, for example, a PD-1 inhibitor, IDO inhibitor, TDO inhibitor, and/or CTLA-4 inhibitor. In additional or further embodiments, the PD-1 inhibitor, IDO inhibitor, TDO/IDO inhibitor, TDO inhibitor, and/or CTLA-4 inhibitor is metabolized upon administration to a subject to produce a metabolite that is then used to produce a desired effect, including a desired therapeutic effect. In some embodiments, a metabolite of an active agent is a derivative of that compound that is formed when the compound is metabolized. The term active metabolite refers to a biologically active derivative of a compound that is formed when the compound is metabolized. In some cases, a prodrug is easier to administer than the parent drug, for example, the prodrug is bioavailable by oral administration whereas the parent is not. In some cases, the prodrug has improved solubility in pharmaceutical compositions over the parent drug. In a non-limiting example, a prodrug of an active agent described herein is administered as an ester prodrug to facilitate transmittal across a cell membrane where water solubility is detrimental to mobility, where the prodrug is metabolically hydrolyzed to the carboxylic acid, the active entity, once inside the cell where water-solubility is beneficial. As a further non-liming example, a prodrug is a short peptide bonded to an acid group, where the peptide is metabolized to reveal the active moiety. In some embodiments, a prodrug is designed to alter the metabolic stability and/or the transport characteristics of an active agent, to mask side effects and/or toxicity, to improve the flavor of an agent, and/or to alter other characteristics or properties of the active. In some embodiments, some of the herein-described compounds are prodrugs for other derivatives or active compounds. In some embodiments, some of the herein-described compounds are formulated as prodrugs.

A therapeutically effective amount of an agent or composition is generally the amount of an agent or composition that is required to relieve to some extent one or more symptoms of a disease being treated (e.g., cancer) and/or the amount that will prevent, to some extent, one or more symptoms of a disease that the host being treated has or is at risk of developing. The terms effective amount or therapeutically effective amount, as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an effective amount for therapeutic uses is the amount of the composition comprising a compound as disclosed herein required to provide a clinically significant decrease in disease symptoms. An appropriate effective amount in any individual case is optionally determined using techniques, such as a dose escalation study.

A unit dosage form, in many instances, refers to physically discrete units suitable as unitary dosages for subjects, wherein each unit comprises a predetermined quantity of a composition comprising an active agent such as an anti-cancer agent, as described herein. In many embodiments, a composition further comprises a pharmaceutically acceptable diluent, carrier or vehicle. The specifications for unit dosage forms depend on the particular composition employed, the route and frequency of administration, the effect to be achieved and the pharmacodynamics associated with the composition in the host.

### Pharmaceutical Formulations

Provided herein are compositions comprising one or more active agents, for example, a TDO/IDO inhibitor, formulated with one or more pharmaceutically acceptable excipients, diluents, carriers and/or adjuvants. In addition, compositions of the disclosure include active agents formulated with one or more pharmaceutically acceptable auxiliary substances. Auxiliary substances, such as pH adjusting and buffering agents, tonicity adjusting agents, stabilizers, wetting agents and the like are readily available to the public. Suitable excipient vehicles for a composition include, for example, water, saline, dextrose, glycerol, ethanol and combinations thereof. In addition, the vehicle may comprise auxiliary substances such as wetting or emulsifying agents or pH buffering agents.

In some embodiments, a composition described herein is administered to a patient using any means capable to result in a desired effect. In some cases, the patient has cancer and the desired effect is tumor cell death. In some cases, the patient has cancer and the desired effect is the prevention of tumor cell growth. In some cases, the patient has cancer and the desired effect is the prevention of tumor cell invasion. In some cases, the patient has cancer and the desired effect is the inhibition of tumor cell invasion. In some cases, the patient has cancer and the desired effect is the prevention of tumor cell metastasis. In some cases, the patient has cancer and the desired effect is the inhibition of tumor cell metastasis.

In many embodiments, the active agent is formulated into a pharmaceutical composition by combination with appropriate, pharmaceutically acceptable carriers or diluents, into solid, semi-solid, liquid or gaseous forms, such as tablets, capsules, powders, granules, ointments, solutions, suppositories, injections, inhalants and aerosols. For oral preparations, the active agent may be used alone or in combination with appropriate additives to make tablets, powders, granules or capsules, for example, with conventional additives, such as lactose, mannitol, corn starch, or potato starch; with binders such as crystalline cellulose, cellulose derivatives, acacia, corn starch or gelatins; with disintegrators, such as corn starch, potato starch, or sodium carboxymethylcellulose; with lubricants, such as talc or magnesium stearate; and/or if desired, with diluents, buffering agents, moistening agents, preservatives and/or flavoring agents.

Compositions described herein, in various implementations, comprise a sustained-release or controlled release matrix. In addition, embodiments of the compositions may be used in conjunction with other treatments that use sustained-release formulations. A sustained-release matrix, in many instances, is a matrix made of materials, usually polymers, which are degradable by enzymatic or acid-based hydrolysis or by dissolution. Once inserted into the body, the matrix may be acted upon by enzymes and body fluids. Examples of sustained-release matrix materials include, without limitation, liposomes, polylactides (polylactic acid), polyglycolide (polymer of glycolic acid), polylactide co-glylide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids (e.g., phenylalanine, tyrosine, isoleucine), polynucleotides, polyvinyl propylene, polyvinylpyrrolidone and silicone. Illustrative biodegradable matrices include a polylactide matrix, a polyglycolide matrix and a polylactide co-glycolide (co-polymers of lactic acid and glycolic acid) matrix.

In some embodiments, an active agent of a composition herein is formulated into a preparation for injection by dissolving, suspending or emulsifying the agent in an aqueous or non-aqueous solvent, such as vegetable or other similar oils, synthetic aliphatic acid glycerides, esters of high aliphatic acids, or propylene glycol; and if desired, with conventional additives such as solubilizers, isotonic agents, suspending agents, emulsifying agents, stabilizers and preservatives.

In some embodiments, an active agent of a composition herein is utilized in an aerosol formulation to be administered via inhalation. As examples, the agent is formulated into a pressurized acceptable propellant such as dichlorodifluoromethane, propane and nitrogen.

In some embodiments, an active agent of a composition herein is made into a suppository by mixing with a base, such as an emulsifying base or water-soluble base. In some instances, an active agent is administered rectally via a suppository. The suppository may include vehicles such as cocoa butter, carbowaxes and polyethylene glycols, which melt at body temperature, yet are solidified at room temperature.

In some embodiments, an active agent of a composition is formulation in an injectable composition. Typically, injectable compositions are prepared as liquid solutions or suspensions. In some instances, a solid form is provided which is suitable for solubilization or suspension in a liquid vehicle prior to injection. In other embodiments, an active agent is emulsified or the active agent is encapsulated in a liposome vehicle.

In some embodiments, unit dosage forms for oral or rectal administration, such as syrups, elixirs and suspensions are provided wherein each dosage unit (e.g., teaspoonful, tablespoonful, table, suppository) comprises a predetermined amount of the composition comprising one or more active agents. In some embodiments, unit dosage forms for injection or intravenous administration comprises the active agent in a composition as a solution in sterile water, normal saline or other pharmaceutically acceptable carrier.

In some embodiments, an active agent of a composition herein is formulated for delivery by a continuous or controlled delivery system. Examples include the use of continuous or controlled delivery devices in combination with catheters, injection devices and the like. In other or additional embodiments, the composition is delivered using a pump, including mechanical and electromechanical infusion pumps. In general, pumps provide consistent and/or controlled release of the composition over time. In some embodiments, the active agent is in a liquid formulation in a drug-impermeable reservoir, and is delivered in a continuous or controlled manner to a patient. In some embodiments, a drug delivery system is at least partially implantable. An implantable device can be implanted at any suitable implantation site using methods and devices well known in the art Implantation sites include, but are not limited to, a subdermal, subcutaneous, intramuscular or other suitable site within a subject's body. Subcutaneous implantation sites are used in some embodiments for convenience in implantation and removal of the drug delivery device. In some embodiments, the active agent is delivered in a controlled release system. In exemplary embodiments, the active agent is administered using intravenous infusion, implantable osmotic pump, transdermal patch or liposomes.

In other embodiments, an active agent of a composition described herein is formulated into absorptive materials, such as sutures, bandages and gauze; or coated onto the surface of solid phase materials, such as surgical staples, zippers and catheters to deliver the agent.

### Methods of Dosing and Treatment Regimens

The compositions comprising one or more active agents described herein may be administered to a patient in one or more doses. In some embodiments, a composition comprises two or more active agents. In some embodiments, a composition comprising one or more active agents is administered with one or more addition compositions, each comprising one or more additional active agents. In some instances, a patient is administered one dose of one active agent and another dose of another active agent. In some embodiments, an active agent is a TDO/IDO inhibitor. In some embodiments, an active agent is a PD-1 inhibitor. In some embodiments, an active agent is an IDO inhibitor. In some embodiments, an active agent is a CTLA-4 inhibitor. In some embodiments, an active agent is a TDO inhibitor. In some cases, the patient has cancer.

In various embodiments, the compositions containing the one or more active agents described herein are administered for prophylactic and/or therapeutic treatments. In some therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial. In some embodiments, the disease is cancer. In some embodiments, the disease is an infectious disease.

In prophylactic applications, compositions containing the one or more active agents described herein are administered to a patient susceptible to or otherwise at risk of a particular disease, disorder or condition. Such an amount is defined to be a prophylactically effective amount or dose. In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a patient who previously experienced at least one symptom of the disease being treated and is currently in remission, a pharmaceutical composition comprising an active agent, or a pharmaceutically acceptable salt of the active agent, in order to prevent a return of the symptoms of the disease or condition. In some embodiments, the disease is cancer. In some embodiments, the disease is an infectious disease.

In certain embodiments wherein the patient's condition does not improve, upon the doctor's discretion the administration of composition comprising the one or more active agents is administered chronically, that is, for an extended period of time, including throughout the duration of the patient's life in order to ameliorate or otherwise control or limit the symptoms of the patient's disease or condition.

In certain embodiments wherein a patient's status does improve, the dose of active agent(s) being administered is temporarily reduced or temporarily suspended for a certain length of time (i.e., a drug holiday). In some embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, for example, by 10%-100%, including only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

In some embodiments, the dose of an active agent administered to a patient varies depending on factors such as time point during therapy, identity of active agent or combination of active agents, identity of disease, disease condition/severity, identity of patient (e.g., age, weight, sex), and route of administration.

In some embodiments, an active agent is administered an amount from about 25 mg to about 100 mg per dose. In some embodiments, an active agent is administered an amount from about 100 mg to about 200 mg per dose. In some embodiments, an active agent is administered an amount from about 200 mg to about 400 mg per dose. In some embodiments, an active agent is administered an amount from about 400 mg to about 500 mg. In some embodiments, an active agent is administered an amount from about 500 mg to about 1,500 mg. In some embodiments, an active agent is a PD-1 inhibitor. In some embodiments, an active agent is an IDO inhibitor. In some embodiments, an active agent is a CTLA-4 inhibitor. In some embodiments, an active agent is a TDO/IDO inhibitor. In some embodiments, an active agent is a TDO inhibitor.

In some embodiments, an active agent is administered in about a 50 mg dosage. In some embodiments, an active agent is administered in about a 100 mg dosage. In some embodiments, an active agent is administered in about a 200 mg dosage. In some embodiments, an active agent is administered in about a 300 mg dosage. In some embodiments, an active agent is administered in about a 400 mg dosage. In some embodiments, an active agent is administered in about a 500 mg dosage. In some embodiments, an active agent is administered in about a 600 mg dosage. In some embodiments, an active agent is administered in about a 700 mg dosage. In some embodiments, an active agent is administered in about a 800 mg dosage. In some embodiments, an active agent is administered in about a 900 mg dosage. In some embodiments, an active agent is administered in about a 1000 mg dosage. In some embodiments, an active agent is administered in about a 1,500 mg dosage. In some embodiments, an active agent is a PD-1 inhibitor. In some embodiments, an active agent is an IDO inhibitor. In some embodiments, an active agent is a CTLA-4 inhibitor. In some embodiments, an active agent is a TDO/IDO inhibitor. In some embodiments, an active agent is a TDO inhibitor.

A composition as described herein, in various embodiments, comprises two or more active agents. In some embodiments, one active agent comprises one or more CTLA-4 inhibitors. In some embodiments, one active agent comprises one or more PD-1 inhibitors. In some embodiments, one active agent comprises one or more IDO inhibitors. In some embodiments, one active agent comprises one or more TDO/IDO inhibitors. In some embodiments, one active agent comprises one or more TDO inhibitors. In some embodiments, a composition comprises two or more TDO/IDO inhibitors. In some embodiments, a composition comprises at least one TDO/IDO inhibitor and at least one PD-1 inhibitor. In some embodiments, a composition comprises at least one TDO/IDO inhibitor and at least one IDO inhibitor. In some embodiments, a composition comprises at least one TDO/IDO inhibitor and at least one CTLA-4 inhibitor. In some embodiments, a composition comprises at least one TDO/IDO inhibitor and at least one TDO inhibitor. In some cases, a PD-1 inhibitor is administered in about a 1 mg to about 2,000 mg dose. In some cases, an IDO inhibitor is administered in about a 1 mg to about 2,000 mg dose. In some cases, a CTLA-4 inhibitor is administered in about a 1 mg to about 2,000 mg dose. In some cases, a TDO/IDO inhibitor is administered in about a 1 mg to about 2,000 mg dose. In some cases, a TDO inhibitor is administered in about a 1 mg to about 2,000 mg dose. In many implementations of the disclosure, the amount of active agent per dose is determined on a per body weight basis. For example, in an embodiment, the active agent is administered in an amount of about 0.5 mg/kg body weight to about 100 mg/kg body weight. In some cases, a PD-1 inhibitor is administered in a dose of about 0.5 mg/kg to about 100 mg/kg. In some cases, an IDO inhibitor is administered in a dose of about 0.5 mg/kg to about 100 mg/kg. In some cases, a CTLA-4 inhibitor is administered in a dose of about 0.5 mg/kg to about 100 mg/kg. In some cases, a TDO/IDO inhibitor is administered in a dose of about 0.5 mg/kg to about 100 mg/kg. In some cases, a TDO inhibitor is administered in a dose of about 0.5 mg/kg to about 100 mg/kg. Those of skill will readily appreciate that dose levels often vary as a function of the specific active agent administered, the severity of the symptoms of an infected patient and the susceptibility of the subject to side effects. Preferred dosage forms of a given compound are readily determinable by those of skill in the art. In some embodiments, the daily dosage or the amount of active agent in the dosage form are lower or higher than the ranges indicated herein, based on a number of variables in regard to an individual treatment regime. In various embodiments, the daily and unit dosages are altered depending on a number of variables including, but not limited to, the activity of the inhibitor used, the disease or condition to be treated, the mode of administration, the requirements of the individual subject, the severity of the disease or condition being treated, and/or the judgment of the practitioner.

In various embodiments, the dose of an active agent in a composition described herein is administered multiple times. The frequency of administration, in some instances, is dependent on the method of use, for example, for treatment of cancer or infectious disease. In some embodiments, an active agent is administered once per month, twice per month, three times per month, every other week, once per week, twice per week, three times per week, four times per week, five times per week, six times per week, every other day, daily, twice a day, three times a day, four times a day, five times a day, six times a day or more. In some embodiments, an active agent is administered continuously.

The duration of administration of the active agent (period of time over which the agent is administered), in many instances, varies depending on a number of factors. Examples of such factors include, without limitation, patient response, severity of symptoms, and disease type (e.g., cancer type). In an example, an active agent is administered over a period of time of about one day to about one week, about one week to about two weeks, about two weeks to about four weeks, about one month to about two months, about two months to about four months, about four months to about six months, about six months to about eight months, about eight months to about 1 year, about 1 year to about 2 years or more.

### Treatment of Cancer

In some embodiments, disclosed herein are compositions comprising one or more TDO/IDO inhibitors for the treatment of a disease such as cancer. In some embodiments, disclosed herein are compositions comprising one or more TDO/IDO inhibitors and one or more additional active agents for the treatment of a disease such as cancer. In some embodiments, disclosed herein are compositions comprising one or more TDO/IDO inhibitors that are administered with one or more additional compositions comprising one or more additional active agents. In some cases, an additional active agent is a PD-1 inhibitor. In some cases, an additional active agent is an IDO inhibitor. In some cases, an additional active agent is a CTLA-4 inhibitor. In some cases, an additional active agent is a TDO inhibitor. In some cases, an additional active agent is an anti-cancer agent. Non-limiting examples of TDO/IDO inhibitors include the compounds of Table 1, Formula (I), Formula (II), Formula (III), Formula (IV), Formula (V), Formula (VI), Formula (VII), Formula (VIII), Formula (IX), and salts and solvates thereof. Non-limiting examples of CTLA-4 inhibitors useful in the compositions and methods described herein include ipilimumab (also known as MDX-010, BMS-734016), tremelimumab, antibody clone 9H10, antibody clone BNI3, Del 60, M9-14 del 55, a compound of Table 3, any CTLA-4 inhibitors described elsewhere herein, and salts and solvates thereof. Non-limiting examples of PD-1 inhibitors useful in the compositions and methods described herein include BMS-936559, MEDI4736, MPDL3280A, MSB0010718C, AMP-224, nivolumab, pembrolizumab, pidilizumab, BMS-986016, MDX1105-01, avelumab, a compound of Table 2, any PD-1 inhibitors described elsewhere herein, and salts and solvates thereof. Non-limiting examples of IDO inhibitors useful in the compositions and methods described herein include indoximod, F001287, NLG919, INCB024360, a compound of Table 1, any IDO inhibitors described elsewhere herein, and salts and solvates thereof.

In some embodiments, cancer refers to an abnormal growth of cells that proliferate in an uncontrolled way and, in some cases, metastasize. Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, skin (melanoma or basal cell cancer)) and hematological tumors (such as the leukemias and lymphomas) at any stage of the disease with or without metastases.

In some embodiments, the cancer is a solid tumor. In some embodiments, the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer. In some embodiments, the cancer is a sarcoma, carcinoma, or lymphoma. In some instances, the cancer is metastatic melanoma. In some instances, the cancer is non-small cell lung cancer. In some instances, the cancer is renal-cell cancer. In some instances, the cancer is prostate cancer. In some instances, the cancer is colorectal cancer. In some instances, the cancer is pancreatic cancer. In some instances, the cancer is cervical cancer. In some instances, the cancer is gastric cancer. In some instances, the cancer is ovarian cancer. In some instances, the cancer is breast cancer.

In some embodiments, the cancer is colon cancer. In some embodiments, the cancer is pancreatic cancer. In some embodiments, the cancer is cutaneous T-cell lymphoma. In some embodiments, the cancer is a glioma. In some embodiments, the cancer is head and neck cancer. In some embodiments, the cancer is hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof.

Additional non-limiting examples of cancers include acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, desmoid tumors, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, Hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, and Wilms tumor.

In some embodiments, a composition disclosed herein, or a pharmaceutically acceptable salt thereof, is used in the treatment of oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, myelodysplastic syndrome, pancreatic cancer, T-cell lymphoma, glioma, head and neck cancer, hepatocarcinoma, leukemia, glioblastoma, colorectal carcinoma, gallbladder, mastocytoma, acute myeloid leukemia, adrenocortical carcinoma, bladder urothelial carcinoma, brain tumor, brain lower grade glioma, breast carcinoma, breast invasive carcinoma, cervical carcinoma, cholangiocarcinoma, cutaneous melanoma, diffuse large B-cell lymphoma, endometrial carcinoma, glioblastoma multiform, H&N squamous cell carcinoma, hepatocellular carcinoma, kidney chromophobe carcinoma, lung carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate carcinoma, rectum adenocarcinoma, rectum carcinoma, renal cell carcinoma, renal papillary cell carcinoma, sarcoma, testicular germ cell tumors, thymoma, thyroid carcinoma, uterine carcinosarcoma, melanoma, uveal melanoma, or a combination thereof.

In some embodiments, a composition disclosed herein, or a pharmaceutically acceptable salt thereof, is used in the treatment of non-small cell lung cancer, pancreatic cancer, breast cancer, ovarian cancer, colorectal cancer, or head and neck cancer.

In some embodiments, a composition disclosed herein, or a pharmaceutically acceptable salt thereof, is used in the treatment of a carcinoma, a tumor, a neoplasm, a lymphoma, a melanoma, a glioma, a sarcoma, or a blastoma.

In some embodiments, the carcinoma comprises adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiated carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In some embodiments, the tumor comprises astrocytic tumors, malignant mesothelial tumors, ovarian germ cell tumor, supratentorial primitive neuroectodermal tumors, Wilm's tumor, pituitary tumors, extragonadal germ cell tumor, gastrinoma, germ cell tumors, gestational trophoblastic tumor, brain tumors, pineal and supratentorial primitive neuroectodermal tumors, pituitary tumor, somatostatin-secreting tumor, endodermal sinus tumor, carcinoids, central cerebral astrocytoma, glucagonoma, hepatic adenoma, insulinoma, medulloepithelioma, plasmacytoma, vipoma, and pheochromocytoma.

In some embodiments, the neoplasm comprises intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, lymphangioleio myomatosis and malignant thymoma.

In some embodiments, the lymphoma comprises nervous system lymphoma, AIDS-related lymphoma, cutaneous T-cell lymphoma, non-Hodgkin's lymphoma, mantle cell lymphoma, follicular lymphoma and Waldenstrom's macroglobulinemia.

In some embodiments, the melanoma comprises acral lentiginous melanoma, superficial spreading melanoma, uveal melanoma, lentigo maligna melanomas, melanoma, intraocular melanoma, adenocarcinoma nodular melanoma, and hemangioma.

In some embodiments, the sarcoma comprises adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma.

In some embodiments, the glioma comprises glioma, brain stem glioma, and hypothalamic and visual pathway glioma.

In some embodiments, the blastoma comprises pulmonary blastoma, pleuropulmonary blastoma, retinoblastoma, neuroblastoma, medulloblastoma, glioblastoma, and hemangiblastomas.

In various aspects of the subject matter provided herein, a composition comprising a TDO/IDO inhibitor and optionally one or more active agents, such as an IDO, PD-1 and/or CTLA-4 inhibitor, further comprises or is administered in combination with an anti-cancer agent and/or anti-cancer treatment.

In some embodiments, anti-cancer agents include one or more of the following abiraterone; abarelix; abraxane, adriamycin; actinomycin; acivicin; aclarubicin; acodazole hydrochloride; acronine; adozelesin; aldesleukin; alemtuzumab; allopurinol; alitretinoin; altretamine; ametantrone acetate; aminoglutethimide; aminolevulinic acid; amifostine; amsacrine; anastrozole; anthramycin; aprepitant; arsenic trioxide; asparaginase; asperlin; azacitidine; azetepa; azotomycin; batimastat; bendamustine hydrochloride; benzodepa; bevacizumab; bexarotene; bicalutamide; bisantrene hydrochloride; bisnafide dimesylate; bizelesin; bleomycin; bleomycin sulfate; bortezomib; brequinar sodium; bropirimine; busulfan; cactinomycin; calusterone; caracemide; carbetimer; carboplatin; carmustine; carubicin hydrochloride; carzelesin; capecitabine; cedefingol; cetuximab; chlorambucil; cirolemycin; cisplatin; cladribine; clofarabine; crisnatol mesylate; cyclophosphamide; cytarabine; dacarbazine; dasatinib; daunorubicin hydrochloride; dactinomycin; darbepoetin alfa; decitabine; degarelix; denileukin diftitox; dexormaplatin; dexrazoxane hydrochloride; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; epoetin alfa; erbulozole; erlotinib hydrochloride; esorubicin hydrochloride; estramustine; estramustine phosphate sodium; etanidazole; etoposide; etoposide phosphate; etoprine; everolimus; exemestane; fadrozole hydrochloride; fazarabine; fenretinide; filgrastim; floxuridine; fludarabine phosphate; fluorouracil; flurocitabine; fosquidone; fostriecin sodium; fulvestrant; gefitinib; gemcitabine; gemcitabine hydrochloride; gemcitabine-cisplatin; gemtuzumab ozogamicin; goserelin acetate; histrelin acetate; hydroxyurea; idarubicin hydrochloride; ifosfamide; iimofosine; ibritumomab tiuxetan; idarubicin; ifosfamide; imatinib mesylate; imiquimod; interleukin I1 (including recombinant interleukin II, or rlL2), interferon alfa-2a; interferon alfa-2b; interferon alfa-n1; interferon alfa-n3; interferon beta-1 a; interferon gamma-1 b; iproplatin; irinotecan hydrochloride; ixabepilone; lanreotide acetate; lapatinib; lenalidomide; letrozole; leuprolide acetate; leucovorin calcium; leuprolide acetate; levamisole; liposomal cytarabine; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; methoxsalen; metoprine; meturedepa; mitindomide; mitocarcin; mitocromin; mitogillin; mitomalcin; mitomycin C; mitosper; mitotane; mitoxantrone hydrochloride; mycophenolic acid; nandrolone phenpropionate; nelarabine; nilotinib; nocodazoie; nofetumomab; nogalamycin; ofatumumab; oprelvekin; ormaplatin; oxaliplatin;oxisuran; paclitaxel; palifermin; palonosetron hydrochloride; pamidronate; pegfilgrastim; pemetrexed disodium; pentostatin; panitumumab; pazopanib hydrochloride; pemetrexed disodium; plerixafor; pralatrexate; pegaspargase; peliomycin; pentamustine; peplomycin sulfate; perfosfamide; pipobroman; piposulfan; piroxantrone hydrochloride; plicamycin; plomestane; pomalidomide, porfimer sodium; porfiromycin; prednimustine; procarbazine hydrochloride; puromycin; puromycin hydrochloride; pyrazofurin; quinacrine; raloxifene hydrochloride; rasburicase; recombinant HPV bivalent vaccine; recombinant HPV quadrivalent vaccine; riboprine; rogletimide; rituximab; romidepsin; romiplostim; safingol; safingol hydrochloride; sargramostim; semustine; simtrazene; sipuleucel-T; sorafenib; sparfosate sodium; sparsomycin; spirogermanium hydrochloride; spiromustine; spiroplatin; streptonigrin; streptozocin; sulofenur; sunitinib malate; talisomycin; tamoxifen citrate; tecogalan sodium; tegafur; teloxantrone hydrochloride; temozolomide; temoporfin; temsirolimus; teniposide; teroxirone; testolactone; thalidomide; thiamiprine; thioguanine; thiotepa; tiazofurin; tirapazamine; topotecan hydrochloride; toremifene; tositumomab and I¹³¹ Iodine tositumomab; trastuzumab; trestolone acetate; tretinoin; triciribine phosphate; trimetrexate; trimetrexate glucuronate; triptorelin; tubulozole hydrochloride; uracil mustard; uredepa; valrubicin; vapreotide; verteporfin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorinostat; vorozole; zeniplatin; zinostatin; zoledronic acid; and zorubicin hydrochloride.

In some embodiments, a composition described herein is used in combination with an anti-emetic agent to treat nausea or emesis, which results from the use of the composition, anti-cancer agent(s) and/or radiation therapy.

In some embodiments, a composition described herein comprises or is administered with a NSAID. NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, and COX-2 specific inhibitors (such as, but not limited to, celecoxib, rofecoxib, valdecoxib, parecoxib, etoricoxib, lumiracoxib, CS-502, JTE-522, L-745,337 and NS398).

In some embodiments, a composition described herein is used in combination with radiation therapy. Radiation therapy is optionally used to treat localized solid tumors, such as cancers of the skin, tongue, larynx, brain, breast, prostate, colon, liver, uterus and/or cervix. It is also optionally used to treat leukemia and lymphoma (cancers of the blood-forming cells and lymphatic system, respectively).

### Kits

Provided herein, in one aspect, are kits which include one or more reagents or devices for the performance of the methods disclosed herein. In some embodiments, the kit comprises a composition as described herein. In some embodiments, the kit comprises one, two or more active agents. In some embodiments, the active agents are formulated together. In other embodiments, the active agents are formulated separately. In some embodiments, the kit comprises a means to administrate a composition comprising one or more active agents as described herein. In some embodiments, one or more of the compositions of a kit comprises one or more compounds of Table 1, Table 2, Table 3, Formula (I), Formula (II), Formula (III), Formula (IV), Formula (V), Formula (VI), Formula (VII), Formula (VIII), Formula (IX), or salts, solvates, or combinations thereof.

In some embodiments, the kit comprises suitable instructions in order to perform the methods of the kit. The instructions may provide information of performing any of the methods disclosed herein, whether or not the methods may be performed using only the reagents provided in the kit. The kit and instructions may require additional reagents or systems.

In some embodiments, many reagents may be provided in a kit of the invention, only some of which should be used together in a particular reaction or procedure.

In some embodiments, a kit provided herein includes a carrier means being compartmentalized to receive in close confinement one or more containers such as vials, tubes, and the like, each of the containers comprising one of the separate elements to be used in a method provided herein.

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disorder that benefit by inhibition of PD-1, inhibition of IDO, inhibition of TDO, and/or inhibition of CTLA-4; or in which PD-1, IDO, TDO, and/or CTLA-4 is a mediator or contributor to the symptoms or cause.

The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes, carrier, package, container, vial and/or tube labels listing contents and/or instructions for use, and package inserts with instructions for use. A set of instructions will also typically be included.

In some embodiments, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a pharmaceutical composition comprising a TDO/IDO inhibitor described herein and optional additional active agent is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The following examples are set forth to illustrate more clearly the principle and practice of embodiments disclosed herein to those skilled in the art and are not to be construed as limiting the scope of any claimed embodiments.

### Example 1

### Computational Assessment of IDO/TDO Inhibitors

Potential IDO/TDO inhibitors were assessed using a computational ligand-based screen. The potential IDO/TDO inhibitors were compared for structural similarity to a database of known IDO and TDO inhibitors. Conformational flexibility of the potential inhibitors was first evaluated, and representative conformers for each compound were generated. Subsequently, each conformer of each compound was evaluated for structural and chemical similarity to each of the known inhibitors. After scoring, the potential IDO/TDO inhibitors in the top 1% of all potential IDO/TDO inhibitors screened were compiled into Table 1.

### Example 2

### In Vitro Assay for TDO/IDO Inhibition

Inhibition of IDO by TDO/IDO test inhibitors was assessed in vitro. A substrate mixture was prepared by mixing equal volumes of 80 mM ascorbic acid diluted in 0.405 M Tris, pH 8.0 and a mixture of 800 uL L-tryptophan, 9,000 units/mL catalase and 40 uM methyene blue. Recombinant TDO or IDO was diluted to 16 ng/uL and 50 uL loaded into wells of a 96-well plate. The reaction was started by the addition of 50 uL substrate mixture. The plate was read in kinetic mode at 321 nm for 5 minutes. The rate of increase of absorbance measured in the absence of a TDO/IDO inhibitor is taken as the maximum rate of enzymatic activity. Active TDO/IDO inhibitors decrease the rate of increased absorbance over time, indicating a reduced rate of conversion of tryptophan to N-formyl-kynurenine in a dose-dependent manner. Table 4 shows the inhibition of TDO and IDO with various TDO/IDO test compounds.

TABLE 4
Inhibition of TDO and IDO with TDO/IDO test compounds.

| | TDO Inhibition Assay | | | IDO Inhibition Assay | | |
|---|---|---|---|---|---|---|
| | | | Dose | | | Dose |
| | Screening | | Resp. | Screening | | Resp. |
| | (Activity, %) | | (IC50, | (Activity, %) | | (IC50, |
| Common Name | 10 μM | 1 μM | nM) | 10 μM | 1 μM | nM) |
| Tioconazole | 28 | 83 | 4160 | 47 | 76 | 9330 |
| Liarozole | 43 | 89 | 9330 | 88 | 99 | 63390 |
| Sertaconazole | 15 | 74 | 4070 | 37 | 63 | 8250 |
| Econazole | 20 | 80 | 5350 | 28 | 71 | 4910 |
| Sulconazole | 17 | 72 | 3510 | 11 | 36 | 740 |
| Miconazole | 16 | 74 | 3700 | 27 | 67 | 4690 |
| Isoconazole | 17 | 58 | 4120 | 53 | 76 | 14610 |
| Itraconazole | 82 | 93 | 46960 | 56 | 72 | 17420 |
| Niclosamide | 62 | 82 | | 75 | 84 | |
| Deferasirox | 69 | 107 | | 85 | 90 | |
| Fenticonazole | −34 | 89 | 2731 | 56 | 85 | |
| Cloconazole | 5 | | 532 | 76 | | |
| Eltrombopag | 5 | | 1047 | 83 | | |

### Example 3

### In Vitro Assay for Cancer Cell Cytotoxicity

TDO/IDO test compounds were incubated with various cancer cell lines to determine the effect of the test compounds on cancer cell death. The CellTiter-Glo Luminescent Cell Viability Assay was used to determine cell cytotoxicity by determining the number of viable cells in culture based on quantitation of ATP, which signals the presence of metabolically active cells. INCBO24360 and Cisplatin were used as control anti-cancer agents. The IC₅₀ values corresponding to each test compound and cell line are shown in Table 5.

TABLE 5
Cancer cell cytotoxicity with TDO/IDO test compounds.

| | IC50 (μM) | | | | | | | |
|---|---|---|---|---|---|---|---|---|
| Cell line | Tioconazole | Sulconazole | Cloconazole | Fenticonzole | Isoconazole | Sertaconazole | INCB024360 | Cisplatin |
| HuT78 | 12.14 | 9.37 | 19.43 | 9.16 | 15.69 | 17.69 | 72.55 | 4.50 |
| (human | | | | | | | | |
| CTCL cell | | | | | | | | |
| line) | | | | | | | | |
| U87MG | 26.14 | 13.56 | 26.67 | 15.64 | 63.92 | 13.89 | NA | 8.95 |
| (human | | | | | | | | |
| glioblastoma) | | | | | | | | |
| SCC4 | 28.20 | 109.39 | 46.23 | 78.78 | 83.83 | NA | NA | 18.95 |
| (human | | | | | | | | |
| squamous | | | | | | | | |
| cell | | | | | | | | |
| carcinoma) | | | | | | | | |
| HT29 (human | 18.15 | 8.74 | 10.68 | 13.93 | 14.82 | 11.91 | 53.70 | 23.16 |
| colorectal | | | | | | | | |
| adenocarcinoma) | | | | | | | | |
| CT26 | 14.04 | NA | 8.06 | 7.60 | 14.78 | 10.94 | 69.92 | 4.11 |
| (murine | | | | | | | | |
| colon) | | | | | | | | |
| Pano2 | 22.15 | 9.35 | 9.61 | 16.07 | 12.61 | 16.10 | 53.52 | 6.40 |
| (murine | | | | | | | | |
| pancreatic) | | | | | | | | |
| GL261 | 26.99 | 61.77 | 20.30 | 15.25 | 15.56 | 30.12 | 48.87 | 9.09 |
| (murine | | | | | | | | |
| glioma) | | | | | | | | |

### Example 4

### In Vitro Assay for IDO Inhibition Via Cell Based Enzymatic Assay

Inhibition of IDO by a TDO/IDO inhibitor described herein is assessed in vitro by a cell based assay. HeLa cells are routinely maintained in minimum essential media with supplements. HeLa cells are seeded in a 96-well plate at a density of 5×10³ and grown overnight. On the following day, human IFNy at 50 ng/mL final concentration and serial dilutions of TDO/IDO inhibitor in a total volume of 200 uL culture medium per well are added to the cells. The plates are incubated for 48 hours and then 140 uL of supernatant per well is transferred to a new 96-well plate. To hydrolyze N-formyl-kynurenine produced by IDO to kynurenine, 10 uL of 6.1 N TCA is mixed into each well with incubation at 50° C. for 30 min The reaction mixture is centrifuged to remove sediments. Supernatant from each well are transferred to a clean 96-well plate and mixed with equal volumes of 2% (w/v) p-dimethylaminobenzaldehyde in acetic acid. Absorbance from kynurenine is measured at 480 nm. L-Kynurenine is used as a standard and is prepared in a series of concentrations (240, 120, 60, 30, 15, 7.5, 3.75,1.87 μM) in HeLa cell culture media and analyzed in the same procedure. The percent inhibition at individual concentrations is determined The data is processed using nonlinear regression to generate IC50 values. The rate of increase of absorbance measured in the absence of a TDO/IDO inhibitor is taken as the maximum rate of enzymatic activity. Active inhibitors decrease the rate of increased absorbance over time in a dose-dependent manner, indicating a reduced rate of conversion of tryptophan to N-formyl-kynurenine.

### Example 5

### Murine Colon Cancer In Vitro Assay

TDO/IDO inhibitor test compounds were tested in co-culture experiments in which CT26 murine colon cancer cells and murine immune cells were seeded together. The experimental conditions were selected to reflect the tumor microenvironment, in which cancer cells and immune cells exist in dynamic interaction. Because immune cell suppression by cancer cells can act through the TDO/IDO pathway, the experiment was designed to determine if TDO/IDO test compounds could reverse the immune suppression, encourage cancer cell killing, either directly or indirectly, and activate immune cells. The following compound effects were assessed: (1) cancer cell killing, (2) CD4 cell survival and activation, by looking at the expression of activation markers CD69+ and CD71+, and (3) CD8 cell survival and activation, by looking at the expression of activation markers CD69+ and CD71+.

CT26 Cell Death

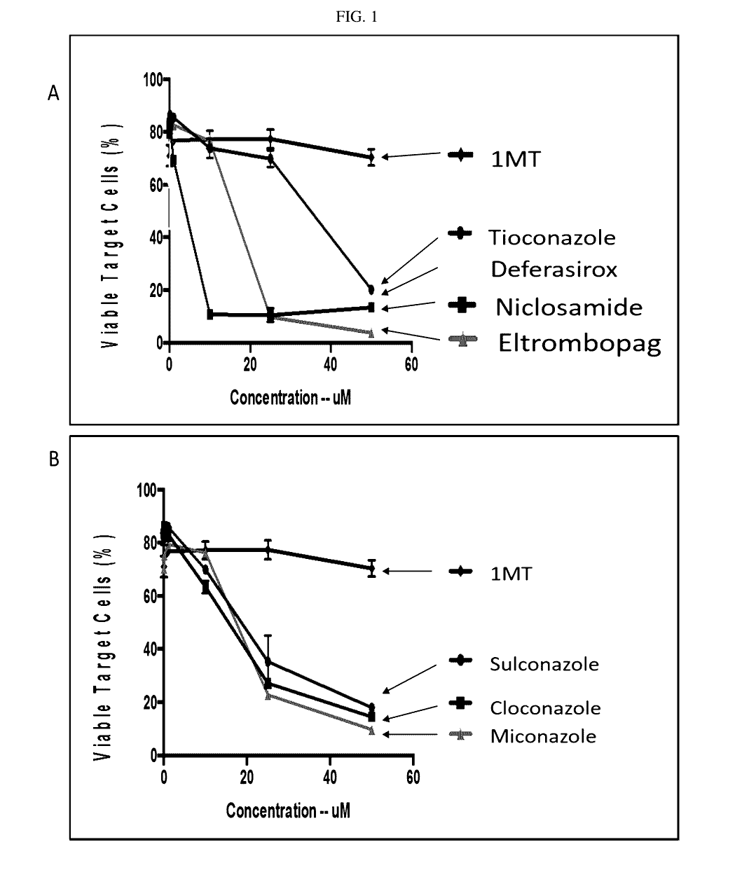

CT26 cell line was maintained in culture as per standard cell culture procedures. Single cell suspension of splenocytes were Balb/c (H2d) mice were prepared; RBCs were lysed using distilled water and 10×PBS and resuspended in complete RPMI medium supplemented with fetal bovine serum, supplements and 2-mercaptoethanol. CT26 cells were harvested using Trypsin/EDTA, first treated with 50 mg/ml Mitomycin C for 20 min at 37° C., washed 3 times in complete medium and then stained with 1 mg/ml of CFSE for one hour at 37° C. TDO/IDO test compounds (tioconazole, niclosamide, eltrombopag, deferasirox, sulconazole, cloconazole, miconazole) were added to final concentrations of 0, 0.1, 1.0, 10.0, 25.0 and 50.0 mM in triplicates in 96 well round-bottom plates. 1-methyl tryptophan (1MT) was used as a control compound. Effector and target cells were seeded at a 50:1 E:T ratio. Plates were incubated for 96 h at 37° C. 1 ml of PI (400 mg/ml) was added to each of the wells and transferred to FACS tubes. Samples were analyzed by FACS on BDFACScan. The frequencies of non-viable target cells (CFSE+PI+) and viable target cells (CFSE+PI−) cells were measured and cell killing reported as the frequency of viable target cells as percentage of total target cell, as shown in FIG. 1, panel A and panel B.

Effector Cell Activation

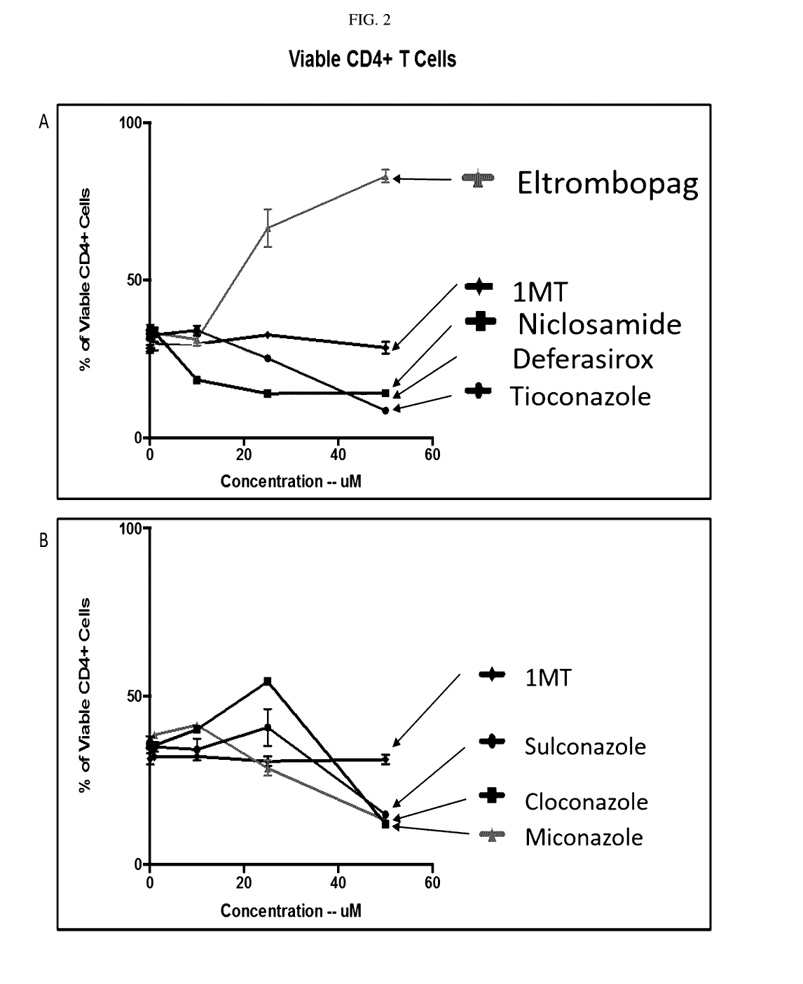

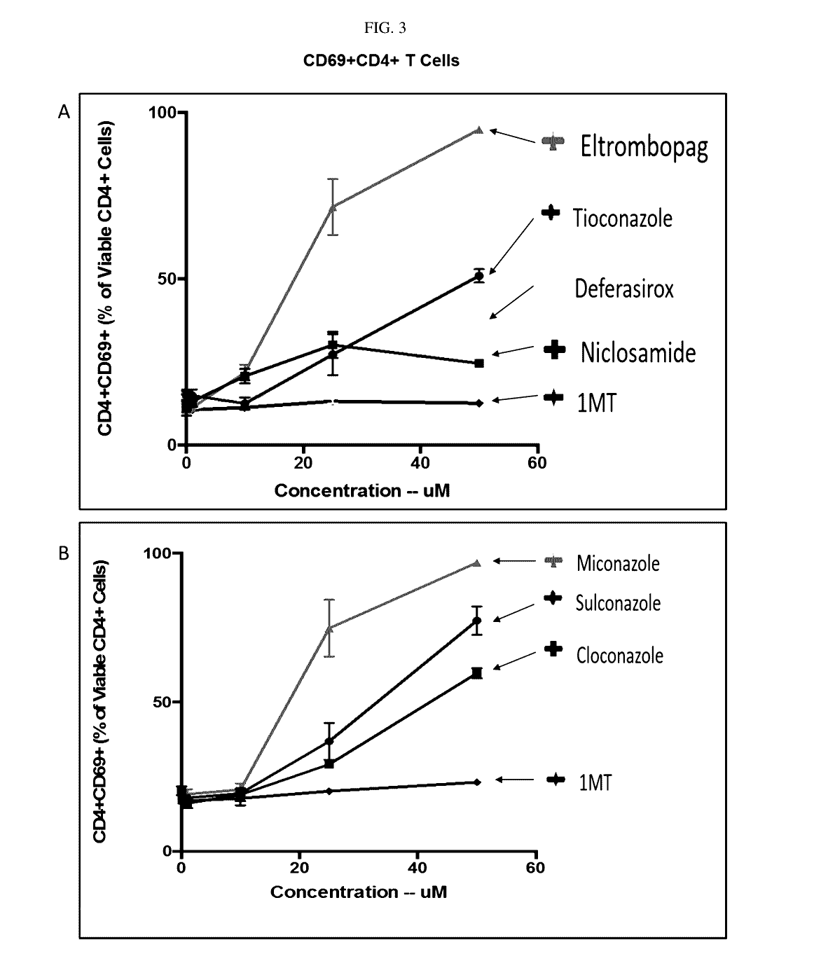

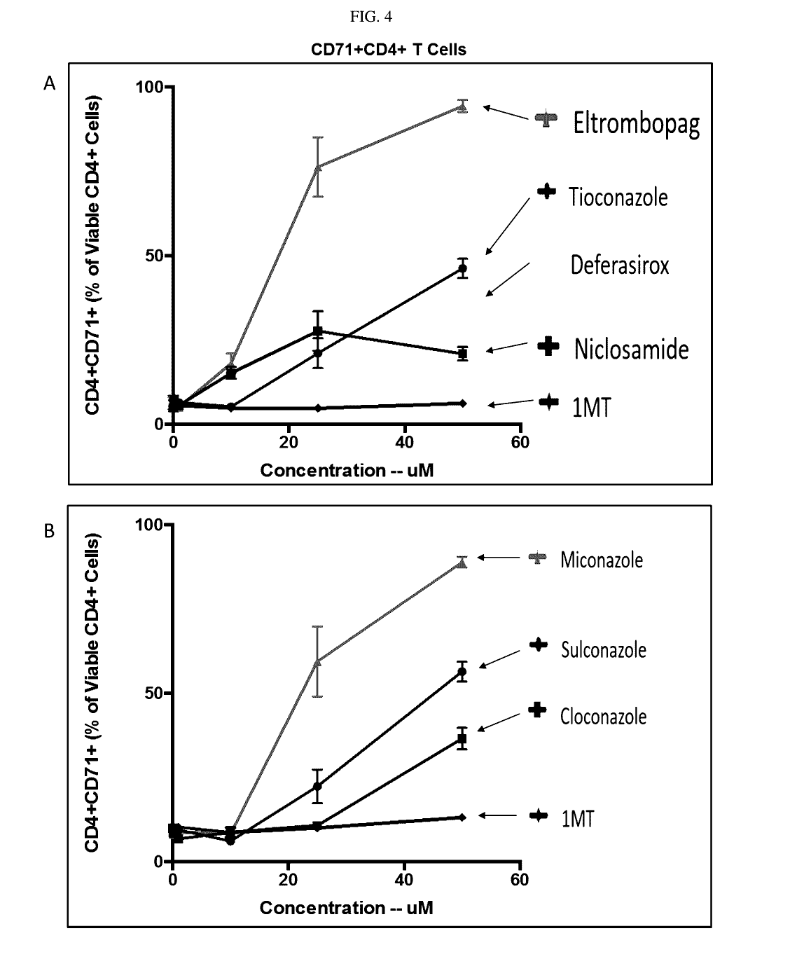

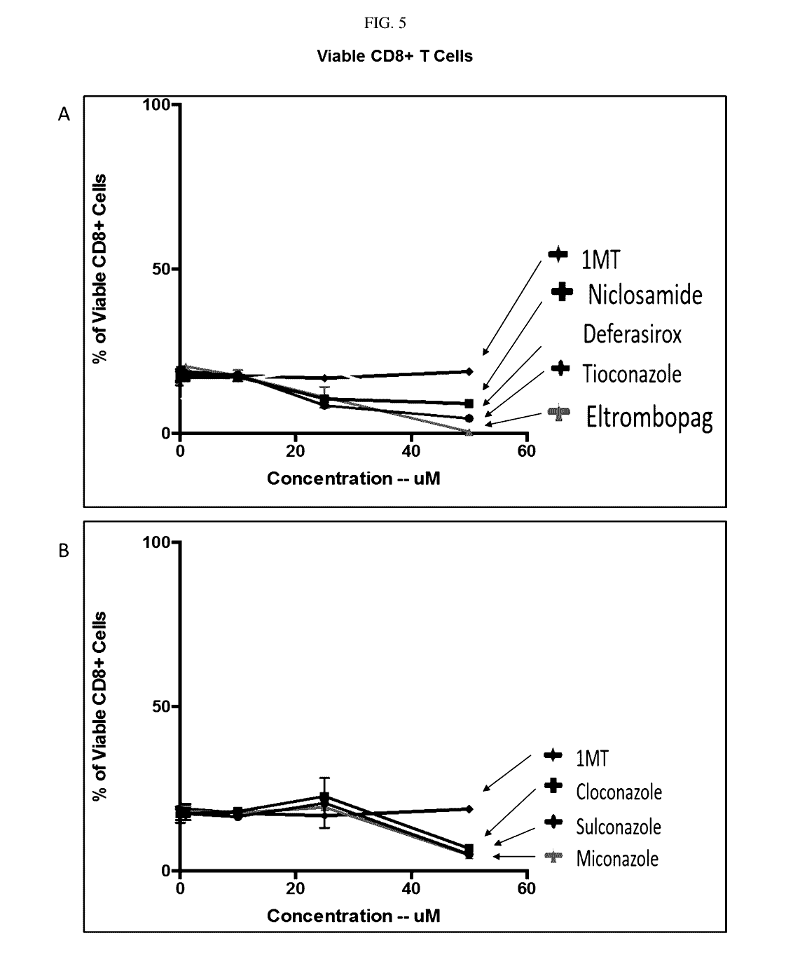

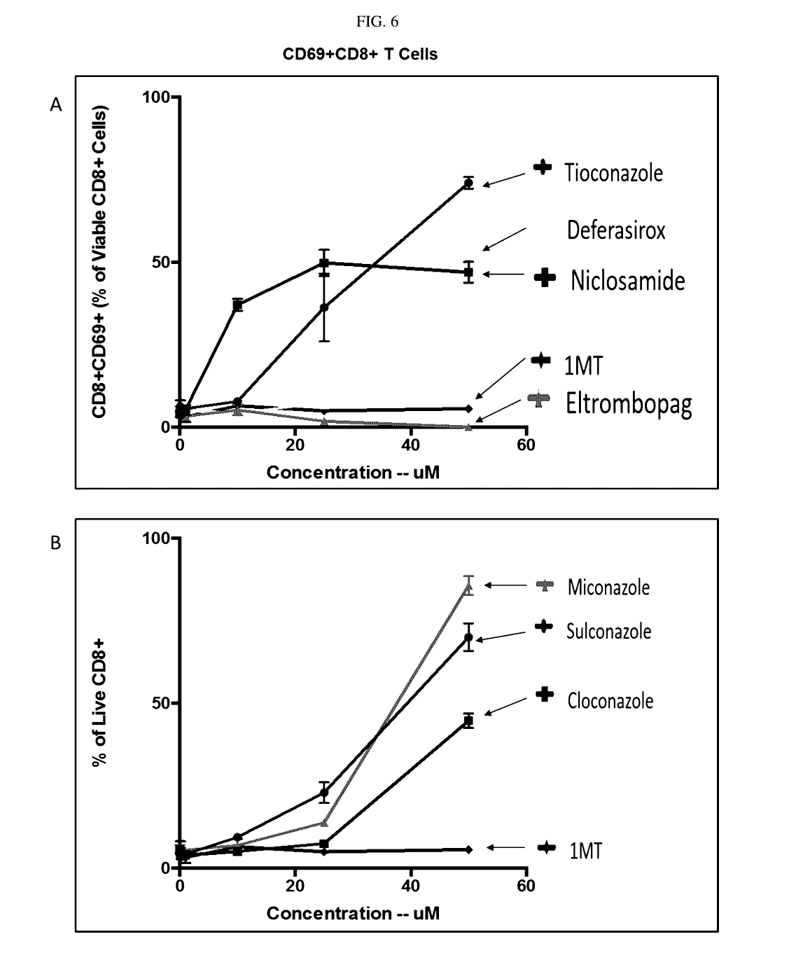

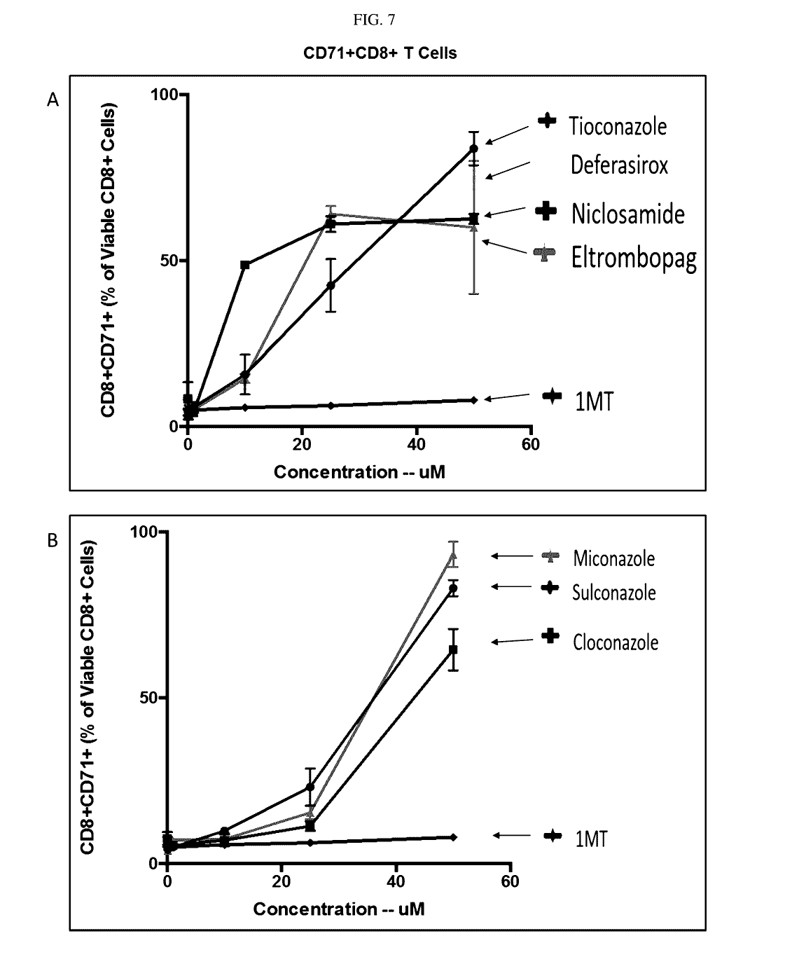

CT26 cell line was maintained in culture as per standard cell culture procedures. Single cell suspension of splenocytes were Balb/c (H2d) mice were prepared; RBCs were lysed using distilled water and 10×PBS and resuspended in complete RPMI medium supplemented with fetal bovine serum, supplements and 2-mercaptoethanol. CT26 cells were harvested using Trypsin/EDTA, treated with 50 mg/ml Mitomycin C for 20 min at 37° C., washed 3 times in complete medium and added to effector cells. TDO/IDO test compounds (tioconazole, niclosamide, eltrombopag, deferasirox, sulconazole, cloconazole, miconazole) were added to final concentrations of 0, 0.1, 1.0, 10.0, 25.0 and 50.0 mM in triplicates in 96 well round-bottom plates. 1-methyl tryptophan was used as a control compound. Effector and target cells were seeded at a 50:1 E:T ratio. Plates were incubated for 96 hrs at 37° C. Cells were harvested into FACS tubes, stained with markers (CD71, CD69, CD4, CD8) and analyzed by FACS on BDFACScan using CellQuest Analysis Software. Data are represented as % of Live cells (based on FSC/SCC gating) for effector cell (CD4, CD8) distribution. Live CD4/CD8 cells were analyzed for activation marker (CD69, CD expression), as shown in panels A and B of FIGS. 2-7.

### Example 6

### Mouse Tumor Model to Evaluate In Vivo Activity of IDO Inhibitors

In vivo activity of TDO/IDO inhibitors described herein is assessed via a mouse tumor model.

Eight week old female C57BL/6 mice are inoculated subcutaneously with 1×10⁶ B16-GMCSF tumor cells. Tumor sizes are measured twice weekly in two dimensions using a caliper, and the volume presented in mm³ using the formula: V=0.5(A×B2), where A and B are the long and short diameters of the tumor, respectively. Tumor bearing animals are sorted into groups with mean tumor volumes of 110-125 mm³. Each day of the study, TDO/IDO inhibitors are reconstituted in 5% DMA, 47.5% propylene glycol. Body weights are monitored throughout the study as a gross measure of toxicity and morbidity. Tumor growth control, expressed in %, is calculated using the formula: 1−[(treated (day X)−treated (day Y))/(vehicle (day X)−vehicle (day Y)], where X is the day of last or interim measurement and Y is the day when dosing commenced.

The average size of tumors in mice untreated with TDO/IDO inhibitors is a positive control. Active IDO inhibitors lead to a decrease in tumor size as compared to the positive control.

### Example 7

### Pharmaceutical Liquid Compositions

To prepare a TDO/IDO inhibitor composition suitable for administration by injection or oral administration, 1 mg to 5,000 mg of a TDO/IDO inhibitor, or a pharmaceutically acceptable salt or solvate thereof, is dissolved in sterile water. Optionally, a sterile saline, a taste masking excipient, and/or a buffer is added to the composition. The TDO/IDO inhibitors formulated in liquid compositions using the methods of this example are listed in Table 1.

To prepare a composition comprising two TDO/IDO inhibitors for administration by injection or oral administration, 1 mg to 5,000 mg of a first a TDO/IDO inhibitor, or a pharmaceutically acceptable salt or solvate thereof, and 1 mg to 5,000 mg of a second a TDO/IDO inhibitor, or a pharmaceutically acceptable salt or solvate thereof, is dissolved in sterile water. Optionally, a sterile saline, a taste masking excipient, and/or a buffer is added to the composition. The TDO/IDO inhibitors formulated in liquid compositions using the methods of this example are listed in Table 1.

To prepare a composition comprising a PD-1 inhibitor and a TDO/IDO inhibitor suitable for administration by injection or oral administration, 1 mg to 5,000 mg of a PD-1 inhibitor, or a pharmaceutically acceptable salt or solvate thereof, and 1 mg to 5,000 mg of a TDO/IDO inhibitor, or a pharmaceutically acceptable salt or solvate thereof, is dissolved in sterile water. Optionally, a sterile saline, a taste masking excipient, and/or a buffer is added to the composition.

To prepare a composition comprising a TDO/IDO inhibitor and a CTLA-4 inhibitor suitable for administration by injection or oral administration, 1 mg to 5,000 mg of a TDO/IDO inhibitor, or a pharmaceutically acceptable salt or solvate thereof, and 1 mg to 5,000 mg of a CTLA-4 inhibitor, or a pharmaceutically acceptable salt or solvate thereof, is dissolved in sterile water. Optionally, a sterile saline, a taste masking excipient, and/or a buffer is added to the composition.

To prepare a composition comprising an IDO inhibitor and a TDO/IDO inhibitor suitable for administration by injection or oral administration, 1 mg to 5,000 mg of an IDO inhibitor, or a pharmaceutically acceptable salt or solvate thereof, and 1 mg to 5,000 mg of a TDO/IDO inhibitor, or a pharmaceutically acceptable salt or solvate thereof, is dissolved in sterile water. Optionally, a sterile saline, a taste masking excipient, and/or a buffer is added to the composition.

To prepare a composition comprising an TDO inhibitor and a TDO/IDO inhibitor suitable for administration by injection or oral administration, 1 mg to 5,000 mg of a TDO inhibitor, or a pharmaceutically acceptable salt or solvate thereof, and 1 mg to 5,000 mg of a TDO/IDO inhibitor, or a pharmaceutically acceptable salt or solvate thereof, is dissolved in sterile water. Optionally, a sterile saline, a taste masking excipient, and/or a buffer is added to the composition.

### Example 8

### Pharmaceutical Oral Dosage

A tablet is prepared by mixing 1 mg to 5 g of a TDO/IDO inhibitor, or a pharmaceutically acceptable salt or solvate thereof, with starch or other suitable powder blend. The mixture is incorporated into an oral dosage unit suitable for oral administration. The TDO/IDO inhibitors formulated in tablet compositions using the methods of this example are listed in Table 1.

A tablet is prepared by mixing 1 mg to 5 g of a first TDO/IDO inhibitor, or a pharmaceutically acceptable salt or solvate thereof, and 1 mg to 5 g of a second TDO/IDO inhibitor, or a pharmaceutically acceptable salt or solvate thereof, with starch or other suitable powder blend. The mixture is incorporated into an oral dosage unit suitable for oral administration. The TDO/IDO inhibitors formulated in tablet compositions using the methods of this example are listed in Table 1.

A tablet is prepared by mixing 1 mg to 5 g of a PD-1 inhibitor, or a pharmaceutically acceptable salt or solvate thereof, and 1 mg to 5 g of a TDO/IDO inhibitor, or a pharmaceutically acceptable salt or solvate thereof, with starch or other suitable powder blend. The mixture is incorporated into an oral dosage unit suitable for oral administration.

A tablet is prepared by mixing 1 mg to 5 g of a TDO/IDO inhibitor, or a pharmaceutically acceptable salt or solvate thereof, and 1 mg to 5 g of a CTLA-4 inhibitor, or a pharmaceutically acceptable salt or solvate thereof, with starch or other suitable powder blend. The mixture is incorporated into an oral dosage unit suitable for oral administration.

A tablet is prepared by mixing 1 mg to 5 g of an IDO inhibitor, or a pharmaceutically acceptable salt or solvate thereof, and 1 mg to 5 g of a TDO/IDO inhibitor, or a pharmaceutically acceptable salt or solvate thereof, with starch or other suitable powder blend. The mixture is incorporated into an oral dosage unit suitable for oral administration.

A tablet is prepared by mixing 1 mg to 5 g of a TDO inhibitor, or a pharmaceutically acceptable salt or solvate thereof, and 1 mg to 5 g of a TDO/IDO inhibitor, or a pharmaceutically acceptable salt or solvate thereof, with starch or other suitable powder blend. The mixture is incorporated into an oral dosage unit suitable for oral administration.

### Example 9

### Clinical Study of TDO/IDO Inhibitor for Subjects with Relapsed or Refractory Solid Tumors

Purpose: The purposes of this study are to assess the efficacy of a TDO/IDO inhibitor as a single agent or in combination, in the treatment of patients with breast cancer, lung cancer, melanoma, pancreatic cancer, solid tumor, collect information on any side effects a TDO/IDO inhibitor may cause as single agent or in combination, and evaluate the pharmacokinetic properties of the compound as single agent or in combination.

Intervention: Patients are administered 5 mg to 500 mg of a TDO/IDO inhibitor as a single agent or in combination in escalating doses. TDO/IDO inhibitors are selected from econazole, sulconazole, isoconazole, miconazole, sertaconazole, tioconazole, fenticonazole, liarozole, cloconazole, itraconazole, niclosamide, deferasirox, and eltrombopag.

Detailed Description: Patients are given a TDO/IDO inhibitor as a single agent or in a combination. Prior to each dosing cycle, a physical exam, blood work and assessment of any side effects are performed. A combination includes a TDO/IDO inhibitor combined with PD-1 inhibitor pidilizumab, IDO inhibitor indoximod, or CTLA-4 inhibitor ipilimumab.

Eligibility and Inclusion Criteria: Male and female subjects that are 18 to 80 years old that have been histopathologically or clinically diagnosed with recurrent or refractory solid tumor malignancy.

Exclusion Criteria: Patients with a psychiatric disorder that might cause difficulty in obtaining informed consent or in conducting the trial; pregnant or nursing; fertile patients must use effective contraception during study treatment; no other non-malignant systemic disease that would preclude drug administration or prolonged follow-up.

Primary Outcome Measures: Determine the safety and efficacy of administration of a single agent or combination; establish toxicities; and define any dose limiting toxicities.

Secondary Outcome Measures: Safety and tolerability by adverse event assessment; duration of overall survival, tumor biomarkers.

### Example 10

### Clinical Study of a TDO/IDO Inhibitor in Combination with Chemotherapy for Subjects with Metastatic Breast Cancer

Purpose: The purposes of this study are to assess the efficacy of a TDO/IDO inhibitor in combination with chemotherapy in the treatment of patients with breast cancer as compared to standard care therapy (chemotherapy alone), collect information on any side effects a TDO/IDO inhibitor may cause in combination, and evaluate the pharmacokinetic properties of the compound in combination.

Intervention

Arm 1: Chemotherapy and placebo

Arm 2: Chemotherapy and a TDO/IDO inhibitor (5 to 500 mg)

Detailed Description: Patients are given a TDO/IDO inhibitor in combination with chemotherapy. Prior to each dosing cycle, a physical exam, blood work and assessment of any side effects are performed. Chemotherapy includes docetaxel, paclitaxel, cisplatin, carboplatin, vinorelbine, capecitabine, liposomal doxorubicin, gemcitabine, mitoxantrone, ixabepilone, nab-paclitaxel and eribulin. TDO/IDO inhibitors are selected from econazole, sulconazole, isoconazole, miconazole, sertaconazole, tioconazole, fenticonazole, liarozole, cloconazole, itraconazole, niclosamide, deferasirox, and eltrombopag.

Eligibility and Inclusion Criteria: Male and female subjects that are 18 to 80 years old that have been histopathologically or clinically diagnosed with metastatic breast cancer; metastatic disease evaluable on imaging; measureable disease having at least one lesion that can be accurately measured; and Eastern Cooperative Oncology Group performance status≦1.

Exclusion Criteria: Patients with a psychiatric disorder that might cause difficulty in obtaining informed consent or in conducting the trial; pregnant or nursing; fertile patients must use effective contraception during study treatment; no other non-malignant systemic disease that would preclude drug administration or prolonged follow-up; patients who have had chemotherapy for the treatment of metastatic breast cancer; patients currently receiving other investigational agents; and patients with active autoimmune disease or condition requiring concurrent use of immunosuppressants.

Primary Outcome Measures: Progression free survival; frequency and grade of adverse events of a TDO/IDO inhibitor and chemotherapy versus chemotherapy with placebo;

Secondary Outcome Measures: Safety and tolerability by adverse event assessment; objective response rate as measured by RECIST 1.1 of a TDO/IDO inhibitor and chemotherapy versus chemotherapy with placebo.

### Example 11

### Clinical Study of a TDO/IDO Inhibitor in Combination with Ipilimumab for Subjects with Metastatic Melanoma

Purpose: The purposes of this study are to assess the efficacy of a TDO/IDO inhibitor in combination with ipilimumab in the treatment of patients with metastatic melanoma as compared to ipilimumab alone, collect information on any side effects a TDO/IDO inhibitor may cause in combination, and evaluate the pharmacokinetic properties of the compound in combination.

Intervention

Arm 1: Ipilimumab is administered intravenously at 3 mg/kg once per 21 day cycle; 4 cycles.

Arm 2: Ipilimumab is administered intravenously at 3 mg/kg once per 21 day cycle; 5-500 mg of a TDO/IDO inhibitor is administered daily on each day of the cycles; 4 cycles; the dose is escalated depending on patient response.

Detailed Description: Patients are given a TDO/IDO inhibitor in combination with ipilimumab at recommended approved dose for treatment of metastatic melanoma. Patients are evaluated clinically and radiographically throughout treatment for tumor evaluation. TDO/IDO inhibitors are selected from econazole, sulconazole, isoconazole, miconazole, sertaconazole, tioconazole, fenticonazole, liarozole, cloconazole, itraconazole, niclosamide, deferasirox, and eltrombopag.

Eligibility and Inclusion Criteria: Male and female subjects that are 18 years and older that have been diagnosed with metastatic melanoma; measureable disease having at least one lesion that can be accurately measured; and Eastern Cooperative Oncology Group performance status≦2.

Exclusion Criteria: Patients with a psychiatric disorder that might cause difficulty in obtaining informed consent or in conducting the trial; pregnant or nursing; fertile patients must use effective contraception during study treatment; no other non-malignant systemic disease that would preclude drug administration or prolonged follow-up; patients currently receiving other investigational agents; patients with previous therapy with CTLA-4 inhibitors; patients with previous therapy with IDO inhibitors; patients with previous therapy with TDO inhibitors; and patients with active autoimmune disease or condition requiring concurrent use of immunosuppressants.

Primary Outcome Measures: Median progression-free survival (PFS); overall incidence of adverse events as a measure of safety and tolerability.

Secondary Outcome Measures: Overall survival; number of participants with adverse events.

What is claimed is:

1. A method of treating cancer comprising administering to an individual in need thereof an effective amount of econazole, sulconazole, isoconazole, miconazole, sertaconazole, tioconazole, fenticonazole, liarozole, cloconazole, itraconazole, niclosamide, deferasirox, eltrombopag, or a pharmaceutically acceptable salt, solvate, or combination thereof.

2. The method of claim 1, provided that econazole or a pharmaceutically acceptable salt or solvate of econazole is administered to the individual.

3. The method of claim 1, provided that sulconazole or a pharmaceutically acceptable salt or solvate of sulconazole is administered to the individual.

4. The method of claim 1, provided that isoconazole or a pharmaceutically acceptable salt or solvate of isoconazole is administered to the individual.

5. The method of claim 1, provided that miconazole or a pharmaceutically acceptable salt or solvate of miconazole is administered to the individual.

6. The method of claim 1, provided that sertaconazole or a pharmaceutically acceptable salt or solvate of sertaconazole is administered to the individual.

7. The method of claim 1, provided that tioconazole or a pharmaceutically acceptable salt or solvate of tioconazole is administered to the individual.

8. The method of claim 1, provided that fenticonazole or a pharmaceutically acceptable salt or solvate of fenticonazole is administered to the individual.

9. The method of claim 1, provided that liarozole or a pharmaceutically acceptable salt or solvate of liarozole is administered to the individual.

10. The method of claim 1, provided that cloconazole or a pharmaceutically acceptable salt or solvate of cloconazole is administered to the individual.

11. The method of claim 1, provided that eltrombopag or a pharmaceutically acceptable salt or solvate of eltrombopag is administered to the individual.

12. The method of claim 1, provided that the cancer comprises colon cancer, pancreatic cancer, cutaneous T-cell lymphoma, glioma, head and neck cancer, hepatocarcinoma, leukemia, glioblastoma, colorectal cancer, gallbladder, mastocytoma, acute myeloid leukemia, adrenocortical cancer, bladder urothelial cancer, brain tumor, brain lower grade glioma, breast cancer, breast invasive cancer, cervical cancer, cholangiocarcinoma, cutaneous melanoma, diffuse large B-cell lymphoma, endometrial cancer, glioblastoma multiform, H&N squamous cell carcinoma, hepatocellular carcinoma, kidney chromophobe carcinoma, lung cancer, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate cancer, rectum adenocarcinoma, rectum cancer, renal cell cancer, renal papillary cell cancer, sarcoma, testicular germ cell tumors, thymoma, thyroid cancer, uterine carcinosarcoma, melanoma, uveal melanoma, or a combination thereof.

13. The method of claim 1, provided that the effective amount is from about 5 mg to about 5,000 mg.

14. The method of claim 1, provided that the individual is administered an additional active agent comprising a PD-1/PD-L pathway inhibitor, IDO inhibitor, TDO inhibitor, CTLA-4 inhibitor, or a combination thereof; or provided that the individual is administered an additional active agent as part of an immunotherapy, CAR T-cell therapy, stem cell therapy, or a combination thereof.

15. A method of inhibiting tryptophan 2,3-dioxygenase (TDO), indoleamine 2,3-dioxygenase (IDO), or both TDO and IDO in an individual in need thereof, the method comprising administering to the individual an effective amount of: an antifungal agent, thrombopoietin (TPO) receptor agonist, or a combination thereof.

16. The method of claim 15, provided that the antifungal agent comprises tioconazole, liarozole, sertaconazole, econazole, sulconazole, miconazole, isoconazole, itraconazole, fenticonazole, cloconazole; or a pharmaceutically acceptable salt, solvate or combination thereof.

17. The method of claim 15, provided that the TPO receptor agonist comprises eltrombopag or a pharmaceutically acceptable salt or solvate of eltrombopag.

18. The method of claim 15, provided that the individual has colon cancer, pancreatic cancer, cutaneous T-cell lymphoma, glioma, head and neck cancer, hepatocarcinoma, leukemia, glioblastoma, colorectal cancer, gallbladder, mastocytoma, acute myeloid leukemia, adrenocortical cancer, bladder urothelial cancer, brain tumor, brain lower grade glioma, breast cancer, breast invasive cancer, cervical cancer, cholangiocarcinoma, cutaneous melanoma, diffuse large B-cell lymphoma, endometrial cancer, glioblastoma multiform, H&N squamous cell carcinoma, hepatocellular carcinoma, kidney chromophobe carcinoma, lung cancer, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, prostate cancer, rectum adenocarcinoma, rectum cancer, renal cell cancer, renal papillary cell cancer, sarcoma, testicular germ cell tumors, thymoma, thyroid cancer, uterine carcinosarcoma, melanoma, uveal melanoma, or a combination thereof.

19. A composition comprising: (a) an antifungal agent, thrombopoietin (TPO) receptor agonist, or a combination thereof; and (b) an anti-cancer agent.

20. The composition of claim 19, provided that the anti-cancer agent comprises a PD-1/PD-L pathway inhibitor, IDO inhibitor, TDO inhibitor, CTLA-4 inhibitor, an immunotherapy agent, a stem cell therapy agent, a CAR T-cell agent, or a combination thereof.
